(19) United States
(12) Patent Application Publication
(10) Pub. No.: US 2017/0144969 A1
(43) Pub. Date: May 25, 2017

(54) ANTIMICROBIAL CONJUGATES, METHOD FOR PRODUCTION AND USES THEREOF

(71) Applicants: The Secretary of State for Health, London (GB); Jawaharlal Nehru Centre for Advanced Scientific Research, Bangalore (IN)

(72) Inventors: Miles Carroll, Salisbury (GB); Jayanta Haldar, Bangalore (IN); Mohini Mohan Konai, Bangalore (IN)

(73) Assignees: The Secretary of State for Health, London (GB); Jawaharlal Nehru Centre for Advanced Scientific Research, Bangalore (IN)

(21) Appl. No.: 15/125,214

(22) PCT Filed: Mar. 13, 2015
(86) PCT No.: PCT/GB2015/050750

§ 371 Date: Sep. 12, 2016

(30) Foreign Application Priority Data
Mar. 13, 2014 (IN) 1345/CHE/2014

(51) Int. Cl. C07D 209/18; C07C 237/10; A61K 31/16; A61K 31/404; A61K 31/40; A61K 31/165; C07C 237/20; C07D 207/16

(52) U.S. Cl. CPC C07D 209/18; C07C 237/20; C07C 237/10; C07D 207/16; A61K 31/404; A61K 31/40; A61K 31/165; A61K 31/16

(57) ABSTRACT

The present disclosure relates to polyamine conjugates, its isomers, prodrugs and pharmaceutically acceptable salts thereof. The present disclosure also relates to process of preparation of polyamine conjugates, its stereoisomers, prodrugs, pharmaceutically acceptable salts thereof, and to pharmaceutical compositions containing them. The compounds of the present disclosure are useful in the treatment, prevention or suppression of diseases mediated by microbes.

### FIELD OF INVENTION

The present disclosure relates to the field of medicinal chemistry and more particularly to the development of antimicrobial compounds. The present disclosure relates to polyamine conjugates, its isomers, prodrugs and pharmaceutically acceptable salts thereof. The present disclosure further relates to the synthesis and characterization of polyamine conjugates to exhibit high antimicrobial activity. The compounds of the present disclosure are useful in the treatment, prevention or suppression of diseases and conditions mediated by microbes.

### BACKGROUND

Bacterial resistance to conventional antibiotics is one of the most serious problems to the global health at this moment. There is ongoing research for the development of novel antibacterial agents to overcome this problem. Antimicrobial Peptides (AMPs) can address such unmet need (Hancock et al. Nat. Biotechnol., 2006, 24, 1551-1557; Hancock et al. Nat. Rev. Drug Discov., 2012, 11, 37-51). AMPs are widely found in the animal and plant kingdom and are usually the first line of defense against any infection (Michael Zasloff Nature, 2002, 45, 389-395). These are amphipathic peptides with net cationic charge and amphiphilicity (hydrophobic/hydrophilic balance) which are known to act primarily by causing lysis of the bacterial cell membrane, while most of the conventional antibiotics have specific targets in the bacteria. Consequently, unlike in the case of conventional antibiotics, where even point mutations can render them inactive, bacteria might have lower propensity to develop resistance against antimicrobial peptides (Brogden et al. Nature Rev. Microbiol., 2005, 3, 238-250). However, no AMP has been approved for clinical use, although some are under promising clinical trials. This is mainly because of the in-vivo toxicity, high cost of manufacture and labile to protease degradation.

Another class of amphipathic peptides is lipopeptides which have a net cationic/anionic charge and lipophilic aliphatic tail. The lipophilic tail in the lipopeptides plays a crucial role towards its activity, which facilitates microbial membrane interaction (Malina et al. Biochem. J., 2005, 390, 695-702; Makovitzki et al. PNAS, 2006, 103, 15997-6002). The lipopeptides generally show antimicrobial activity towards a narrow spectrum of microorganism. For example, daptomycin is only active against Gram-positive bacteria; polymixin (polymixin B and colistin) is active only toward Gram-negative bacteria, while echinocandins function only as antifungal drugs. Daptomycin acts by calcium (Ca⁺²) dependent dissipation of bacterial membrane potential, causes disruption of multiple aspects of cellular function and leakage of cell components. Cationic lipopeptide polymixin binds with the anionic lipopolysaccharide molecules by displacing calcium (Ca⁺²) and magnesium (Mg⁺²) from the outer cell membrane of gram-negative bacteria which leads to permeability changes in the cell envelope resulting in leakage of cell components, and finally causes cell death. FDA has approved daptomycin for treatment of complicated skin and skin structure infections. But recently the resistance of daptomycin has reported (Infect. Dis. Clin. Pract., 2013, 21, 79-84). Polymixin became available for clinical use in the 1960s, but presently it is used only as a last resort because of its toxicity. Limitation of lipopeptides mainly lies in their in-vivo toxicity, structural complexity, challenging synthesis, high cost of manufacture. Hence, substantial effort has been directed towards development of designs and strategies to counter the problems faced by lipopeptides.

From the foregoing it is clear that compounds used in the state of the art to treat and prevent bacterial infection have been found to have limited effect. Further, there is a continuing need to identify new compounds which possess improved antibacterial activity, which have less potential for developing resistance, which possess improved effectiveness against bacterial infections that resist treatment with currently available antibiotics, or which possess unexpected selectivity against target microorganisms.

Spermidine is a polyamine compound (C₇H₁₉N₃) found in ribosomes and living tissues, and having various metabolic functions within organisms. Norspermidine is a homologue of spermidine and possess significant antitumor activity. It also acts as a biofilm-disassembly factor.

Norspermidine interacts directly and specifically with exopolysaccharide. D-amino acids and norspermidine act together to break down existing biofilms (“A Self-produced Trigger for Biofilm Disassembly that Targets Exopolysaccharide”, Koldkin-Gal et. al., Cell, 2012).

A library of polyamine-peptide conjugates is identified to be inhibitors of trypanothione reductase. Norspermidine-based peptides inhibit trypanothione reductase (“Mechanism and structure-activity relationships of norspermidine-based peptidic inhibitors of trypanothione reductase”, Dixon et. al., Biorganic & Medicinal Chemistry, 2005).

WO2012151554 discloses methods of treating or reducing biofilms, treating a biofilm related disorder, and preventing biofilm formation using polyamines. It discloses the use of a combination of norspermidine with D-Tyr or with a mixture of D-Met, D-Trp, D-Leu and D-Tyr to prevent biofilm formation by Bacillus subtilis at concentrations that were ineffective in blocking biofilm formation when applied separately.

WO2013017714 discloses compounds that act as low molecular weight organogels which are structurally similar to Phenylalanine based lipopeptides.

Any agent used to prevent infection or to impair biofilm must be non-toxic towards mammalian cells and safe to the environment. Certain biocidal agents, in quantities sufficient to interfere with biofilms, also can damage host tissues. Antibiotics introduced into local tissue areas can induce the formation of resistant organisms which can then form biofilm communities whose planktonic microorganisms would likewise be resistant to the particular antibiotics. Furthermore, long term systemic antibiotic therapy to eradicate infection causes increase level of toxicity towards host cell. Thus, there is a need to identify and/or develop new compounds and/or derivatives that has enhanced activity against bacterial strains including multidrug resistant bacteria while the compounds are non-toxic and biodegradable in nature.

In addition to antibacterial compounds, there also exists a great need for compounds which are effective against other types of microbe, particularly viruses, fungi and protozoa. The great need for antiviral compounds is exemplified by the current outbreak Ebola virus epidemic, for which there are currently no licensed therapeutics.

### SUMMARY

The need for further antimicrobial compounds (i.e. compounds which are effective against bacteria, viruses, fungi and/or protozoa) is addressed by the present invention.

The present invention is based on the surprising discovery that compounds of formula I (see below) exhibit advantageous anti-microbial properties. Thus, the present disclosure provides a compound of formula I

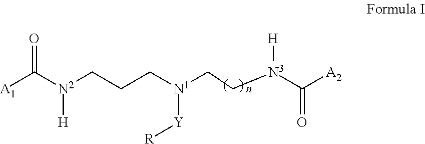

or its stereoisomers, pharmaceutically acceptable salts, polymorphs, solvates and hydrates thereof, wherein, n is 1, 2, 3, 4, or 5; Y is selected from the group of —CH₂—, —CO—, —CONH—, —COO—, A₁ and A₂ are independently selected from a sequence of one or up to 4 additional amino acids, wherein the amino acids are independently selected from L-configuration or D-configuration, wherein A₁ and A₂ are optionally substituted with one or more of R₂; R is selected from C4-C28alkyl, C4-28 alkenyl, C4-C28alkyne, C6-C18aryl, C3-C28cycloalkyl, saturated or unsaturated 5 to 18 membered heterocyclyl, with 1, 2 or 3 hetero atoms selected from O, N and S, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

The present disclosure further relates to a compound of formula I or its stereoisomers, pharmaceutically acceptable salts, polymorphs, solvates and hydrates thereof, for use in killing or inhibiting the growth of a microorganism selected from the group consisting of bacteria, virus, fungi and protozoa. In one embodiment, the microorganism is bacteria or virus. In one embodiment, the microorganism is bacteria. In one embodiment, the microorganism is virus. In one embodiment, the microorganism is bacteria. In one embodiment, the microorganism is fungus. In one embodiment, the microorganism is protozoa. In one embodiment, the virus is Ebola virus.

The present disclosure further relates to use of a compound of formula I or its stereoisomers, pharmaceutically acceptable salts, polymorphs, solvates and hydrates thereof, in killing or inhibiting the growth of a microorganism selected from the group consisting of bacteria, virus, fungi and protozoa. In one embodiment, the microorganism is bacteria or virus. In one embodiment, the microorganism is bacteria. In one embodiment, the microorganism is virus. In one embodiment, the microorganism is bacteria. In one embodiment, the microorganism is fungus. In one embodiment, the microorganism is protozoa. In one embodiment, the virus is Ebola virus.

The present disclosure further relates to a compound of formula I or its stereoisomers, pharmaceutically acceptable salts, polymorphs, solvates and hydrates thereof, for use in treating a disease or condition in a patient wherein said disease or condition is caused by a microorganism selected from the group consisting of bacteria, virus, fungi and protozoa. In one embodiment, the microorganism is bacteria or virus. In one embodiment, the microorganism is bacteria. In one embodiment, the microorganism is virus. In one embodiment, the microorganism is bacteria. In one embodiment, the microorganism is fungus. In one embodiment, the microorganism is protozoa. In one embodiment, the virus is Ebola virus.

The present disclosure further relates to use of a compound of formula I or its stereoisomers, pharmaceutically acceptable salts, polymorphs, solvates and hydrates thereof, in treating disease or condition in a patient, wherein said disease or condition is caused by a microorganism selected from the group consisting of bacteria, virus, fungi and protozoa. In one embodiment, the microorganism is bacteria or virus. In one embodiment, the microorganism is bacteria. In one embodiment, the microorganism is virus. In one embodiment, the microorganism is bacteria. In one embodiment, the microorganism is fungus. In one embodiment, the microorganism is protozoa. In one embodiment, the virus is Ebola virus.

The present disclosure further relates to a method of treating a disease or condition in a patent, said method comprising administering to a patient a compound of formula (I), as claimed in any one of the claims 1 to 6, or its stereoisomers, pharmaceutically acceptable salts, polymorphs, solvates and hydrates thereof, wherein said disease or condition is caused by microorganism selected from the group consisting of bacteria, virus, fungi and protozoa.

Diseases and conditions caused by bacteria, virus, fungi and protozoa are well known in the art.

The patient is a typically a mammal, preferably a human.

In some embodiments, treatment of disease or condition caused by microorganism comprises administration of a therapeutically effective amount of compound to a patient. A therapeutically effective amount refers to the amount of the compound, which when administered alone or in combination to a patient, is sufficient to affect such treatment. An appropriate therapeutically effective amount in any given instance may be ascertained by those skilled in the art or capable of determination by routine experimentation. A therapeutically effective amount is also one in which any toxic or detrimental effects of the compound are outweighed by the beneficial effects.

The present disclosure further relates to a compound of formula I or its stereoisomers, pharmaceutically acceptable salts, polymorphs, solvates and hydrates thereof, for treating diseases caused by bacteria, fungi, and virus.

The present disclosure further relates to a compound of formula I or its stereoisomers, pharmaceutically acceptable salts, polymorphs, solvates and hydrates thereof, for preparing antimicrobial coatings and/or surfaces with or without pharmaceutical compositions.

The present disclosure relates to a composition comprising a compound of formula (I) or a salt thereof, and a carrier.

The present disclosure relates to a pharmaceutical composition comprising a compound of formula I or a pharmaceutically acceptable salt thereof, together with a pharmaceutically acceptable carrier, optionally in combination with one or more other pharmaceutical compositions.

The present disclosure relates to a process of preparation of compound of formula I or its stereoisomers, pharmaceutically acceptable salts, polymorphs, solvates and hydrates thereof.

These and other features, aspects, and advantages of the present subject matter will become better understood with reference to the following description. This summary is provided to introduce a selection of concepts in a simplified form. This summary is not intended to identify key features or essential features of the disclosure, nor is it intended to be used to limit the scope of the subject matter.

### BRIEF DESCRIPTION OF DRAWINGS

FIG. 1 shows antibacterial efficacy in 50% human plasma.

FIG. 2 shows cytotoxicity against HeLa cell line: (a) Cell viability at different concentrations of compound 16, (b) Brightfield microscopic images of cells at different conditions (scale bar=100 μm).

FIG. 3 shows bactericidal kinetics of compound 16 against stationary-phase S. aureus.

FIG. 4 shows propensity to induce bacterial resistance: comparison of fold of increase in MIC of compound 16 and control antibiotics norfloxacin and colistin.

FIG. 5 shows biofilm disruption of S. aureus: (a) Quantification of cell viability in biofilms, (b) Visualization by crystal-violet staining and (c) Confocal microscopy images of biofilms.

FIG. 6 shows calculation of concentrations for compound 16 preparation.

### DETAILED DESCRIPTION

In the structural formulae given herein and throughout the present disclosure, the following terms have been indicated meaning, unless specifically stated otherwise.

### DEFINITIONS

The term “alkyl” refers to a monoradical branched or unbranched saturated hydrocarbon chain having from 4 to 28 carbon atoms, more preferably 6 to 20 carbon atoms. This term is exemplified by groups such as n-butyl, iso-butyl, t-butyl, n-hexyl, n-decyl, tetradecyl, and the like. The groups may be optionally substituted.

The term “alkenyl” refers to a monoradical of a branched or unbranched unsaturated hydrocarbon group preferably having from 4, 5, 6, 7, 8, 9, 10, 11, 12, 13, 14, 15, 16, 17, 18, 19, 20, 21, 22, 23, 24, 25, 26, 27, or 28 carbon atoms, more preferably 6, 7, 8, 9, 10, 11, 12, 13, 14, 15, 16, 17, 18, 19, or 20 carbon atoms and even more preferably 12, 13, 14, 15 or 16 carbon atoms and having 1, 2, 3, 4, 5 or 6 double bond (vinyl), preferably 1 double bond. The groups may be optionally substituted.

The term “alkyne” refers to a monoradical of an unsaturated hydrocarbon, preferably having from 4, 5, 6, 7, 8, 9, 10, 11, 12, 13, 14, 15, 16, 17, 18, 19, 20, 21, 22, 23, 24, 25, 26, 27, or 28 carbon atoms, more preferably 6, 7, 8, 9, 10, 11, 12, 13, 14, 15, 16, 17, 18, 19, or 20 carbon atoms and even more preferably 12, 13, 14, 15 or 16 carbon atoms and having 1, 2, 3, 4, 5 or 6 sites of acetylene (triple bond) unsaturation, preferably 1 triple bond. The groups may be optionally substituted.

“Halo” or “Halogen”, alone or in combination with any other term means halogens such as chloro (Cl), fluoro (F), bromo (Br) and iodo (I).

The term “aryl” refers to an aromatic carbocyclic group of 6 to 18 carbon atoms having a single ring (e.g. phenyl) or multiple rings (e.g. biphenyl), or multiple condensed (fused) rings (e.g. naphthyl or anthranyl). Preferred aryls include phenyl, naphthyl and the like. The groups may be optionally substituted.

The term “cycloalkyl” refers to carbocyclic groups of from 3 to 28 carbon atoms having a single cyclic ring or multiple condensed rings which may be partially unsaturated. Such cycloalkyl groups include, by way of example, single ring structures such as cyclopropyl, cyclobutyl, cyclopentyl, cyclopentenyl, cyclohexyl, cyclohexenyl, cyclooctyl, and the like, or multiple ring structures such as adamantanyl, bicyclo[2.2.1]heptane, 1,3,3-trimethylbicyclo[2.2.1]hept-2-yl, (2,3,3-trimethylbicyclo[2.2.1]hept-2-yl), or carbocyclic groups to which is fused an aryl group, for example indane, and the like. The groups may be optionally substituted.

The term “heterocyclyl” refers to a saturated or partially unsaturated group or unsaturated group having a single ring or multiple condensed rings, having from 5 to 18 carbon atoms and from 1 to 10 hetero atoms, preferably 1, 2, or 3 heteroatoms, selected from nitrogen, sulfur, phosphorus, and/or oxygen within the ring. Heterocyclic groups can have a single ring or multiple condensed rings, and include tetrahydrofuranyl, morpholinyl, piperidinyl, piperazinyl, dihydropyridinyl, tetrahydroquinolinyl, pyrrolidinyl and the like. The groups may be optionally substituted.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

The compounds described herein may contain one or more chiral centers and/or double bonds and therefore, may exist as stereoisomers, such as double-bond isomers (i.e., geometric isomers), regioisomers, enantiomers or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds may also exist in several tautomeric forms including the enol form, the keto form and mixtures thereof. Accordingly, the chemical structures depicted herein encompass all possible tautomeric forms of the illustrated or identified compounds.

“Pharmaceutically acceptable salt” embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

The term “polymorphs” refers to crystal forms of the same molecule, and different polymorphs may have different physical properties such as, for example, melting temperatures, heats of fusion, solubilities, dissolution rates and/or vibrational spectra as a result of the arrangement or conformation of the molecules in the crystal lattice.

The term “solvate”, as used herein, refers to a crystal form of a substance which contains solvent.

The term “hydrate” refers to a solvate wherein the solvent is water.

The term “drug sensitive bacterium” as used herein is a bacterium which is not able to survive exposure to at least one drug.

The present disclosure relates to a compound of formula I

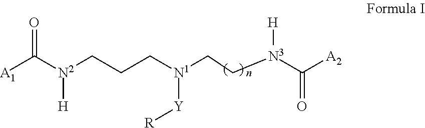

or its stereoisomers, pharmaceutically acceptable salts, polymorphs, solvates and hydrates thereof, wherein, n is 1, 2, 3, 4, or 5; Y is selected from the group of —CH₂—, —CO—, —CONH—, —COO—; A₁ and A₂ are independently selected from a sequence of one or up to 4 additional amino acids, wherein the amino acids are independently selected from L-configuration or D-configuration, wherein A₁ and A₂ are optionally substituted with one or more of R₂; R is selected from C4-C28alkyl, C4-C28alkenyl, C4-C28 alkyne, C6-C18aryl, C3-C28 cycloalkyl, saturated or unsaturated 5 to 18 membered heterocyclyl, with 1, 2 or 3 hetero atoms selected from O, N and S, wherein R is optionally substituted with one or more of R₂; R₂ is independently selected from the group consisting of hydrogen, halogen, CF₃, CN, straight or branched C1-C6 alkyl, C3-C6 cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I

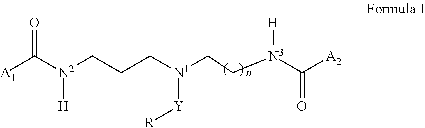

or its stereoisomers, pharmaceutically acceptable salts, polymorphs, solvates and hydrates thereof wherein, n is 1, 2, 3, 4, or 5; Y is selected from the group of —CH₂—, —CO—, —CONH—, —COO—; A₁ and A₂ are independently selected from a sequence of one or up to 4 additional amino acids, wherein the amino acids are independently selected from L-configuration or D-configuration, wherein A₁ and A₂ are independently positively charged; R is selected from C4-C28alkyl, C4-C28alkenyl, C4-C28 alkyne, C6-C18 aryl, C3-C8cycloalkyl, saturated or unsaturated 5 to 18 membered heterocyclyl, with 1, 2 or 3 hetero atoms selected from O, N and S, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Phenylalanine, wherein A₁ and A₂ are optionally substituted with one or more of R₂; R is C9 alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein n is 1, 2, 3, 4, or 5; Y is selected from the group of —CH₂—, —CO—, —CONH—, —COO—; A₁ and A₂ are same or different, and independently selected from 1, 2, 3, or 4 amino acid residues wherein the amino acid residues are independently selected from L-configuration or D-configuration and are positively charged; R is selected from C4-C28alkyl, C4-C28alkenyl, C4-C28 alkyne, C6-C18 aryl, C3-C8cycloalkyl, saturated or unsaturated 5 to 18 membered heterocyclyl, with 1, 2 or 3 hetero atoms selected from O, N and S, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof, wherein, n is 1, 2, 3, 4, or 5; Y is —CH₂—; A₁ and A₂ are independently selected from a sequence of one or up to 4 additional amino acids, wherein the amino acids are independently selected from L-configuration or D-configuration, wherein A₁ and A₂ are optionally substituted with one or more of R₂; R is selected from C4-C28alkyl, C4-C28alkenyl, C4-C28 alkyne, C6-C18aryl, C3-C28 cycloalkyl, saturated or unsaturated 5 to 18 membered heterocyclyl, with 1, 2 or 3 hetero atoms selected from O, N and S, wherein R is optionally substituted with one or more of R₂; R₂ is independently selected from the group consisting of hydrogen, halogen, CF₃, CN, straight or branched C1-C6 alkyl, C3-C6 cycloalkyl, C5-C6 aryl, aromatic 5 to 6 membered heterocyclyl, with 1, 2 or 3 hetero atoms selected from O, N and S; N¹ is positively charged.

According to an embodiment, the present disclosure relates to a compound of formula I or its stereoisomers, pharmaceutically acceptable salts, polymorphs, solvates and hydrates thereof wherein, n is 1, 2, 3, 4, or 5; Y is —CH₂— or —CO—; A₁ and A₂ are same or different, and independently selected from 2 amino acid residues, wherein the amino acids are independently selected from L-configuration or D-configuration; R is selected from C4-28 alkyl, C6-18 aryl.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Phenylalanine, wherein A₁ and A₂ are optionally substituted with one or more of R₂; R is C11alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Phenylalanine, wherein A₁ and A₂ are optionally substituted with one or more of R₂; R is C13alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Phenylalanine, wherein A₁ and A₂ are optionally substituted with one or more of R₂; R is C15 alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Phenylalanine, wherein A₁ and A₂ are optionally substituted with one or more of R₂; R is C17 alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are D-Phenylalanine, wherein A₁ and A₂ are optionally substituted with one or more of R₂; R is C11alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Tryptophan, wherein A₁ and A₂ are optionally substituted with one or more of R₂; R is C13alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Tryptophan, wherein A₁ and A₂ are optionally substituted with one or more of R₂; R is C15alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof, wherein n is 2; Y is —CO—; A₁ and A₂ are L-Lysine, wherein A₁ and A₂ are optionally substituted with one or more of R₂; R is C9alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Lysine, wherein A₁ and A₂ are optionally substituted with one or more of R₂; R is C11alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Lysine, wherein A₁ and A₂ are optionally substituted with one or more of R₂; R is C13 alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Lysine, wherein A₁ and A₂ are optionally substituted with one or more of R₂; R is C15 alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Lysine, wherein A₁ and A₂ are optionally substituted with one or more of R₂; R is C17 alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Ornithine, wherein A₁ and A₂ are optionally substituted with one or more of R₂; R is C13alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Ornithine, wherein A₁ and A₂ are optionally substituted with one or more of R₂; R is C15alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are D-Lysine, wherein A₁ and A₂ are optionally substituted with one or more of R₂; R is C13alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are D-Lysine, wherein A₁ and A₂ are optionally substituted with one or more of R₂; R is C15alkyl, wherein R is optionally substituted with one or more of R₂; R₂ is independently selected from the group consisting of hydrogen, halogen, CF₃, CN, straight or branched C1-C6alkyl, C3-C6 cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CH₂—; A₁ and A₂ are L-Phenylalanine, wherein A₁ and A₂ are optionally substituted with one or more of R₂; R is C7 alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CH₂—; A₁ and A₂ are L-Alanine, wherein A₁ and A₂ are optionally substituted with one or more of R₂; R is C7alkyl, wherein R is optionally substituted with one or more of R₂; R₂ is independently selected from the group consisting of hydrogen, halogen, CF₃, CN, straight or branched C1-C6alkyl, C3-C6 cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof, wherein n is 2; Y is —CH₂—; A₁ and A₂ are L-Lysine, wherein A₁ and A₂ are optionally substituted with one or more of R₂; R is C7 alkyl, wherein R is optionally substituted with one or more of R₂; R₂ is independently selected from the group consisting of hydrogen, halogen, CF₃, CN, straight or branched C1-C6alkyl, C3-C6 cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof, wherein n is 2; Y is —CO—; A₁ and A₂ are L-Phenylalanine, wherein A₁ and A₂ are optionally substituted with one or more of R₂; R is C10 aryl, wherein R is optionally substituted with one or more of R₂; R₂ is independently selected from the group consisting of hydrogen, halogen, CF₃, CN, straight or branched C1-C6alkyl, C3-C6 cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof, wherein n is 2; Y is —CO—; A₁ and A₂ are L-Lysine, wherein A₁ and A₂ are positively charged; R is C9alkyl, wherein R is optionally substituted with one or more of R₂; R₂ is independently selected from the group consisting of hydrogen, halogen, CF₃, CN, straight or branched C1-C6alkyl, C3-C6 cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Lysine, wherein A₁ and A₂ are positively charged; R is C11 alkyl, wherein R is optionally substituted with one or more of R₂; R₂ is independently selected from the group consisting of hydrogen, halogen, CF₃, CN, straight or branched C1-C6 alkyl, C3-C6 cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Lysine, wherein A₁ and A₂ are positively charged; R is C13alkyl, wherein R is optionally substituted with one or more of R₂; R₂ is independently selected from the group consisting of hydrogen, halogen, CF₃, CN, straight or branched C1-C6alkyl, C3-C6 cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Lysine, wherein A₁ and A₂ are positively charged; R is C15 alkyl, wherein R is optionally substituted with one or more of R₂; R₂ is independently selected from the group consisting of hydrogen, halogen, CF₃, CN, straight or branched C1-C6 alkyl, C3-C6 cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Lysine, wherein A₁ and A₂ are positively charged; R is C17alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Ornithine, wherein A₁ and A₂ are positively charged; R is C13 alkyl, wherein R is optionally substituted with one or more of R₂; R₂ is independently selected from the group consisting of hydrogen, halogen, CF₃, CN, straight or branched C1-C6 alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are L-Ornithine, wherein A₁ and A₂ are positively charged; R is C15alkyl, wherein R is optionally substituted with one or more of R₂; R₂ is independently selected from the group consisting of hydrogen, halogen, CF₃, CN, straight or branched C1-C6 alkyl, C3-C6 cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are D-Lysine, wherein A₁ and A₂ are positively charged; R is C13 alkyl, wherein R is optionally substituted with one or more of R₂; R₂ is independently selected from the group consisting of hydrogen, halogen, CF₃, CN, straight or branched C1-C6alkyl, C3-C6 cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CO—; A₁ and A₂ are D-Lysine, wherein A₁ and A₂ are positively charged; R is C15alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof, wherein n is 2; Y is —CH₂—; A₁ and A₂ are L-Lysine, wherein A₁ and A₂ are positively charged; R is C7 alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S.

According to an embodiment, the present disclosure relates to a compound of formula I or its stereoisomers, pharmaceutically acceptable salts, polymorphs, solvates and hydrates thereof wherein, n is 2; Y is —CH₂—; A₁ and A₂ are L-Phenylalanine, wherein A₁ and A₂ are optionally substituted with one or more of R₂; R is C7 alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S; N¹ is positively charged.

According to an embodiment, the present disclosure relates to a compound of formula I or its stereoisomers, pharmaceutically acceptable salts, polymorphs, solvates and hydrates thereof wherein, n is 2; Y is —CH₂—; A₁ and A₂ are L-Alanine, wherein A₁ and A₂ are optionally substituted with one or more of R₂; R is C7 alkyl, wherein R is optionally substituted with one or more of R₂; R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S; N¹ is positively charged.

According to an embodiment, the present disclosure relates to a compound of formula I or its stereoisomers, pharmaceutically acceptable salts, polymorphs, solvates and hydrates thereof, wherein n is 2; Y is —CH₂—; A₁ and A₂ are L-Lysine, wherein A₁ and A₂ are optionally substituted with one or more of R₂; R is C7 alkyl, wherein R is optionally substituted with one or more of R₂; R₂ is independently selected from the group consisting of hydrogen, halogen, CF₃, CN, straight or branched C1-C6 alkyl, C3-C6 cycloalkyl, C5-C6 aryl, aromatic 5 to 6 membered heterocyclyl, with 1, 2 or 3 hetero atoms selected from O, N and S; N¹ is positively charged.

According to an embodiment, the present disclosure relates to a compound of formula I or its stereoisomers, pharmaceutically acceptable salts, polymorphs, solvates and hydrates thereof wherein, the compound is selected from a group consisting of:

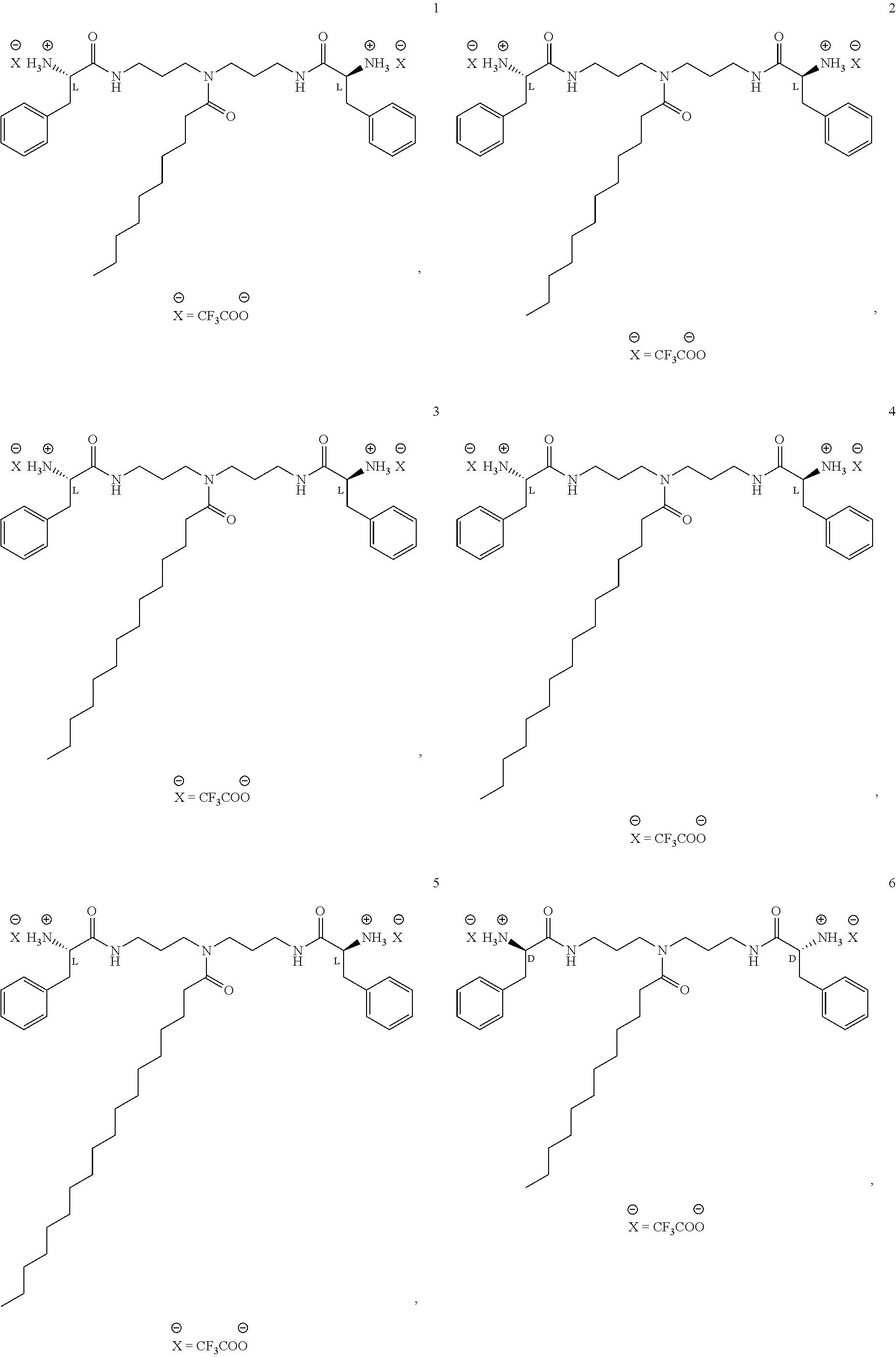

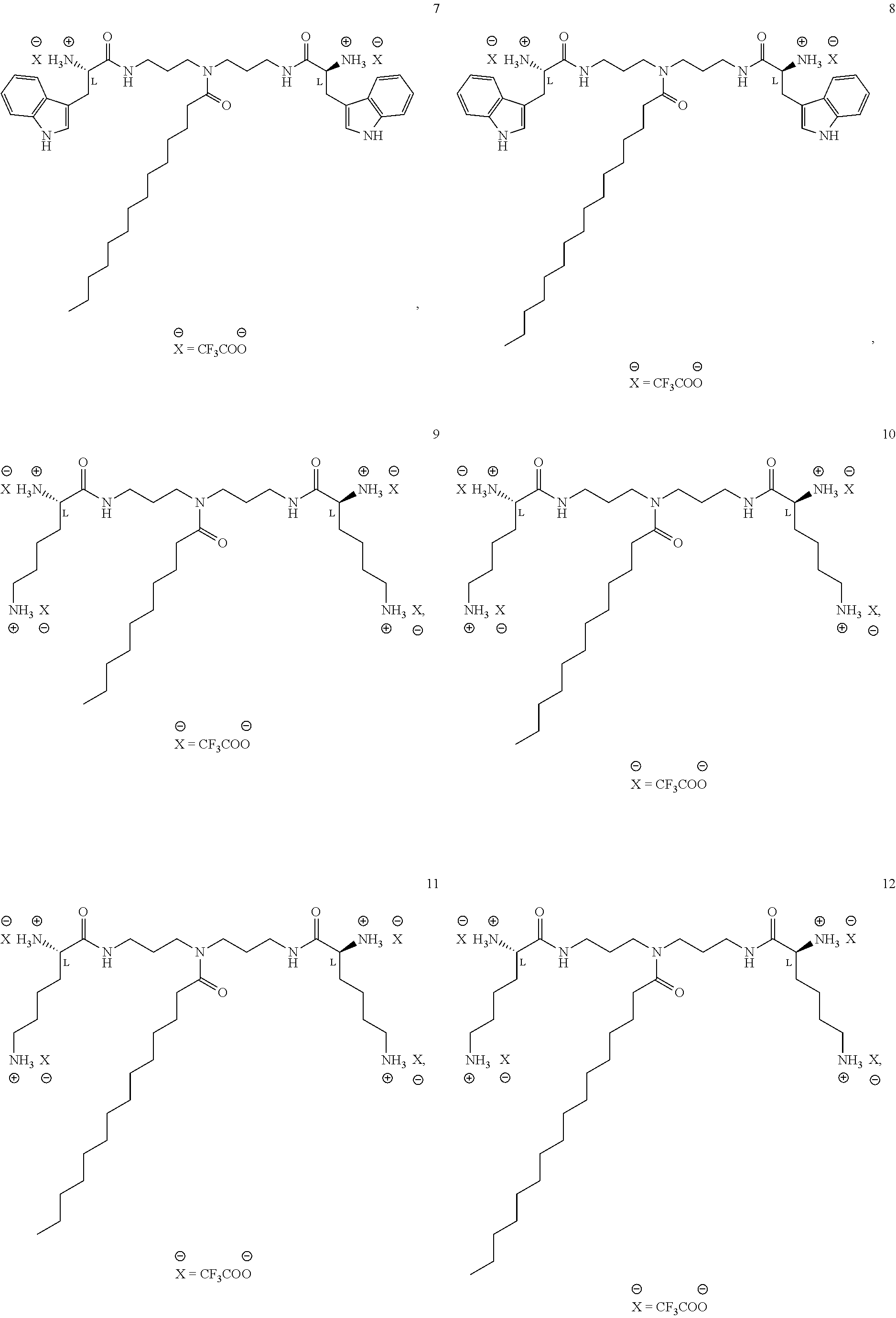

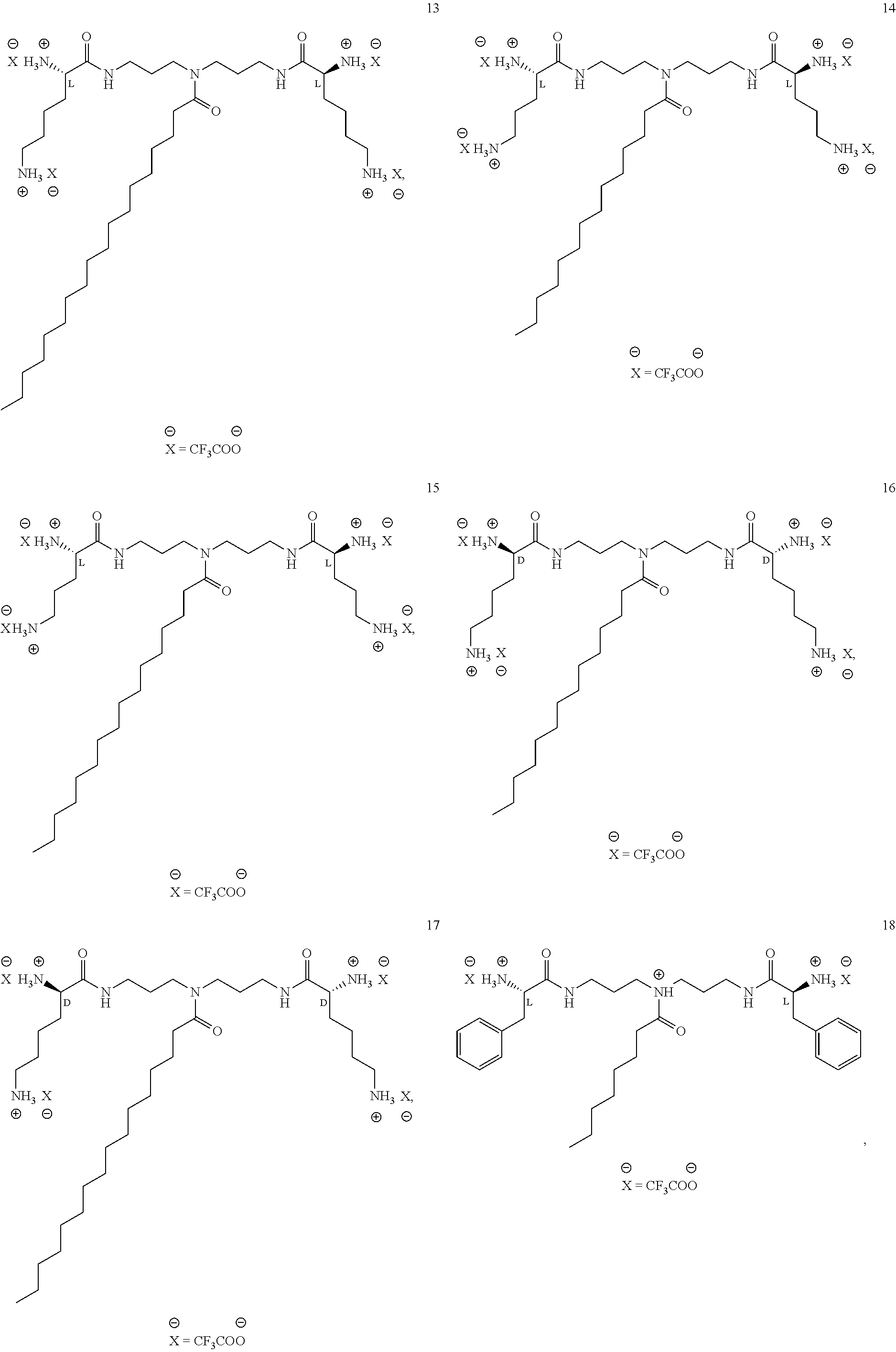

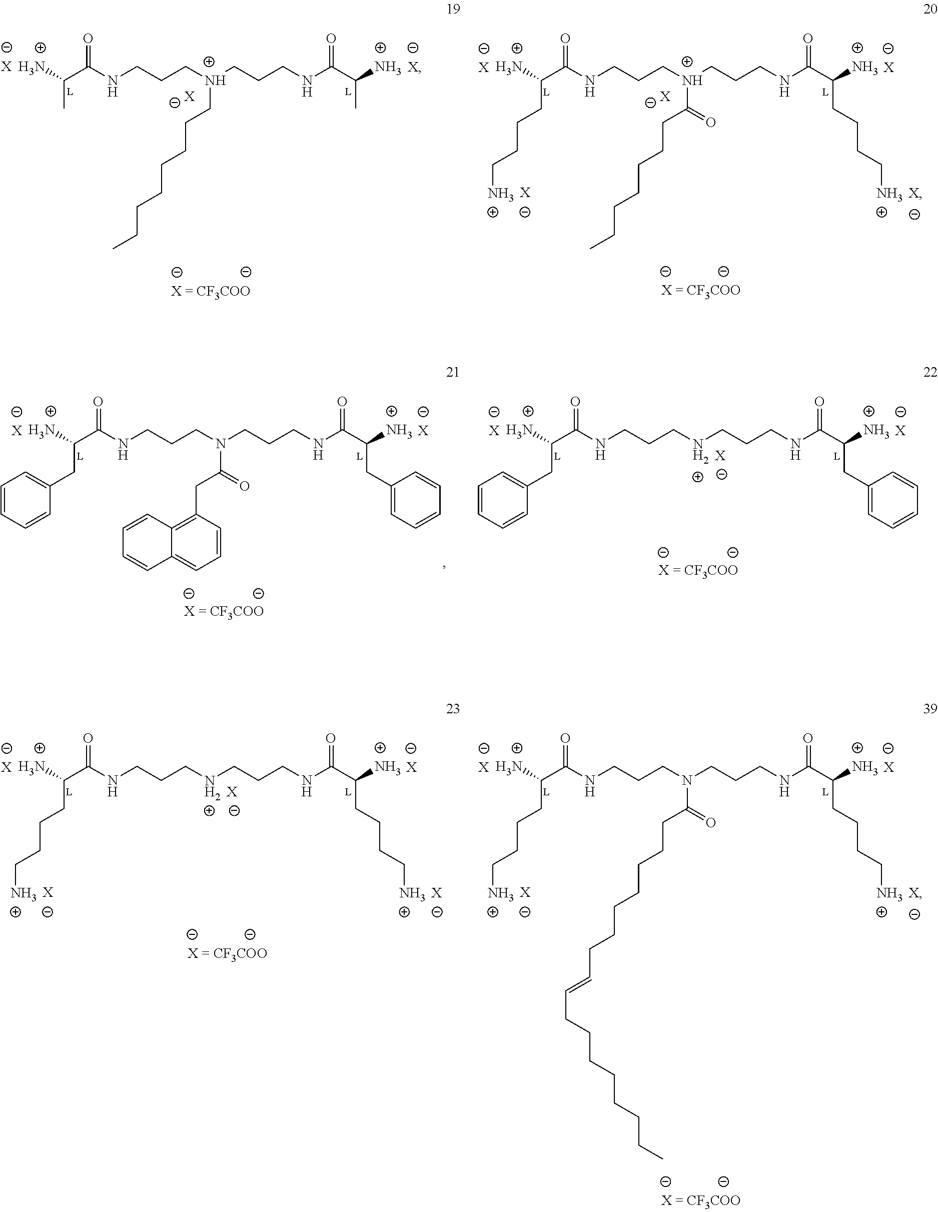

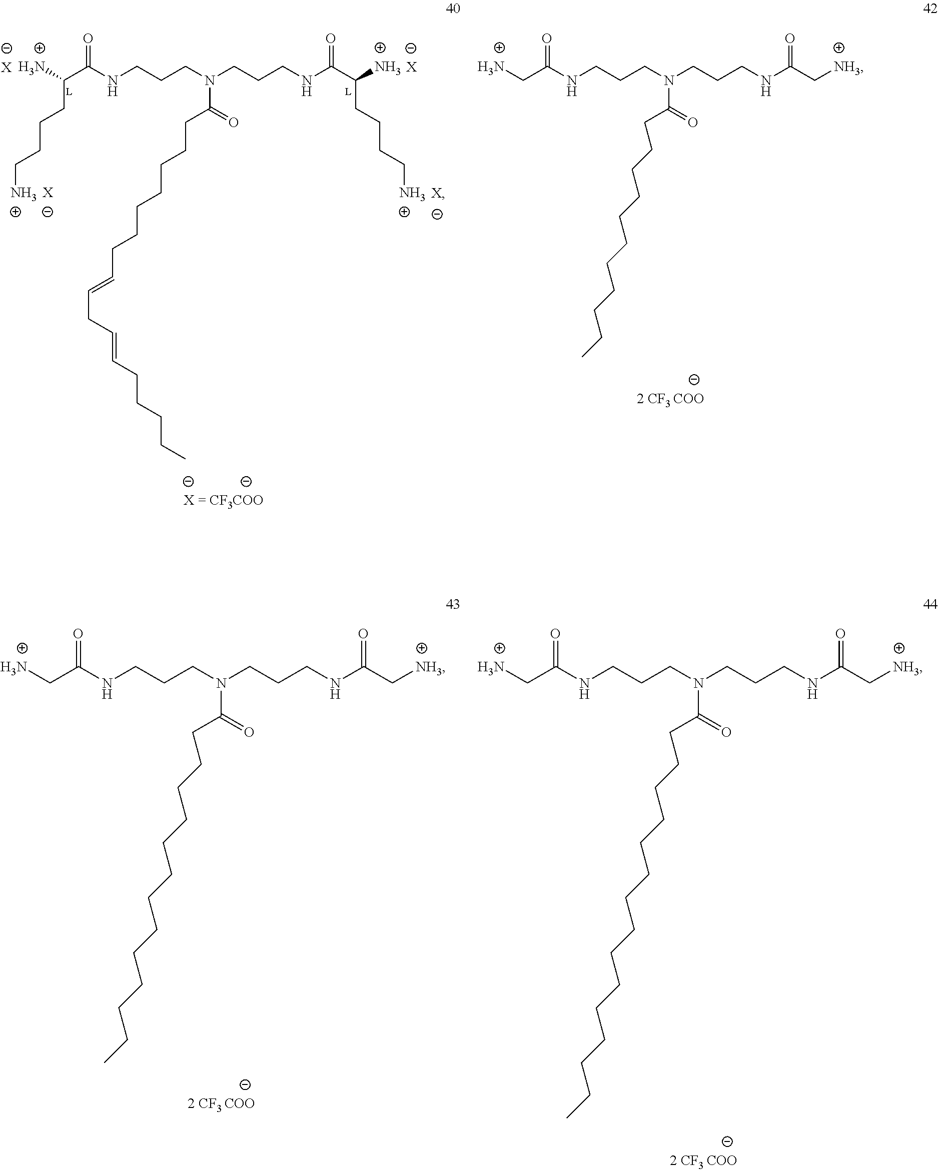

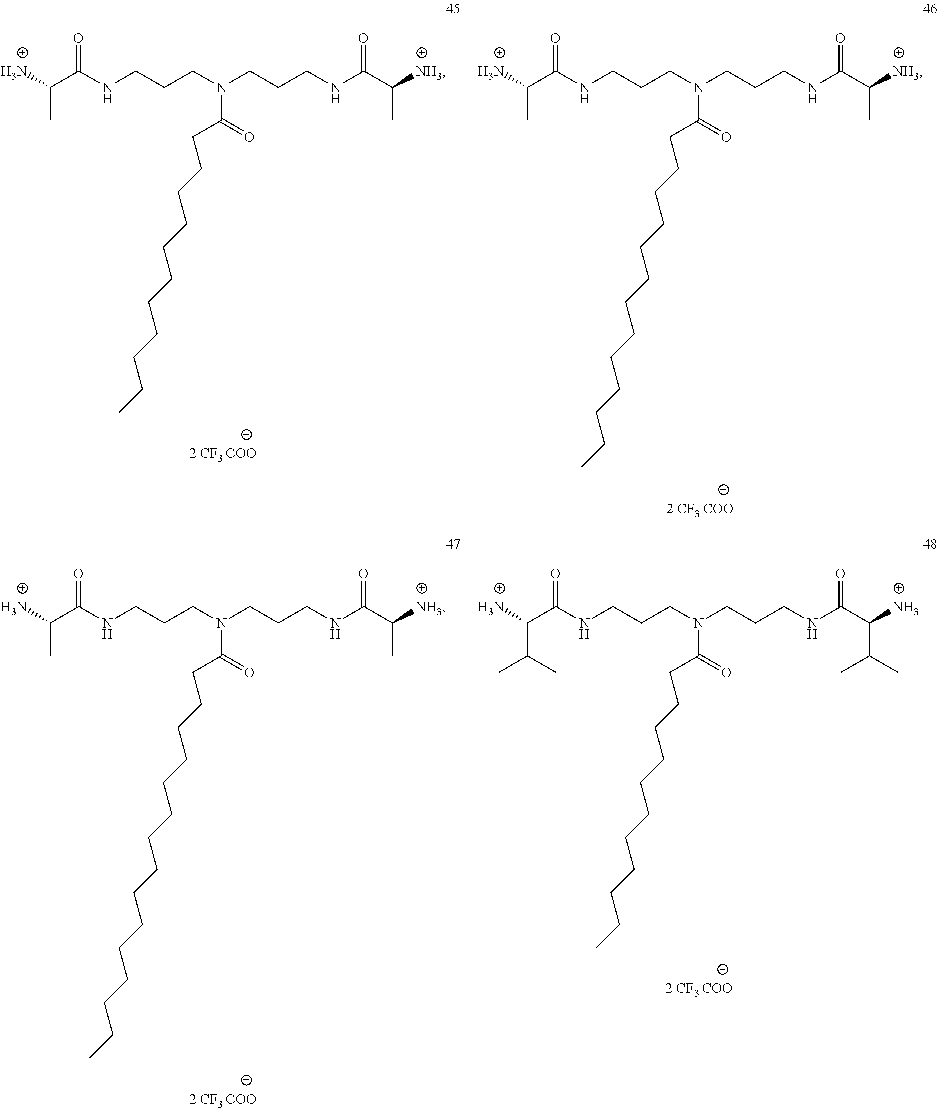

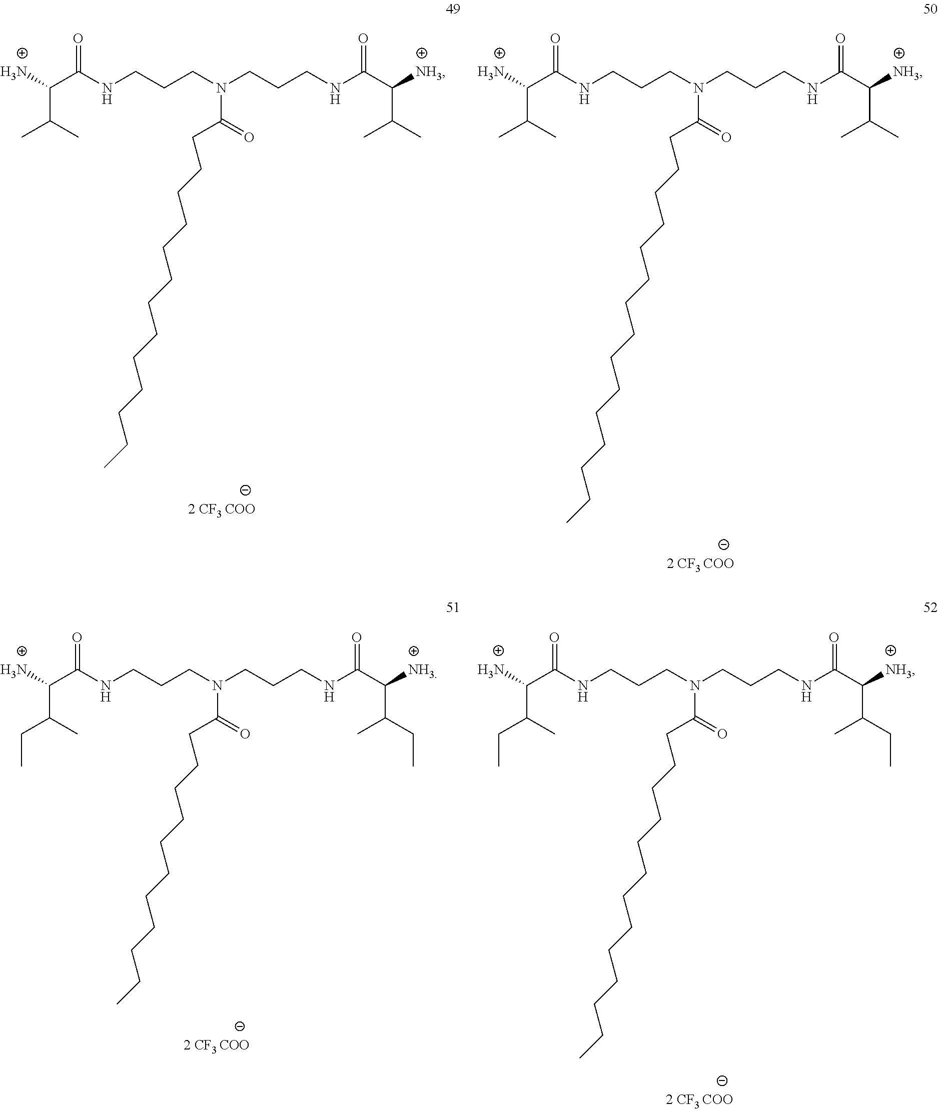

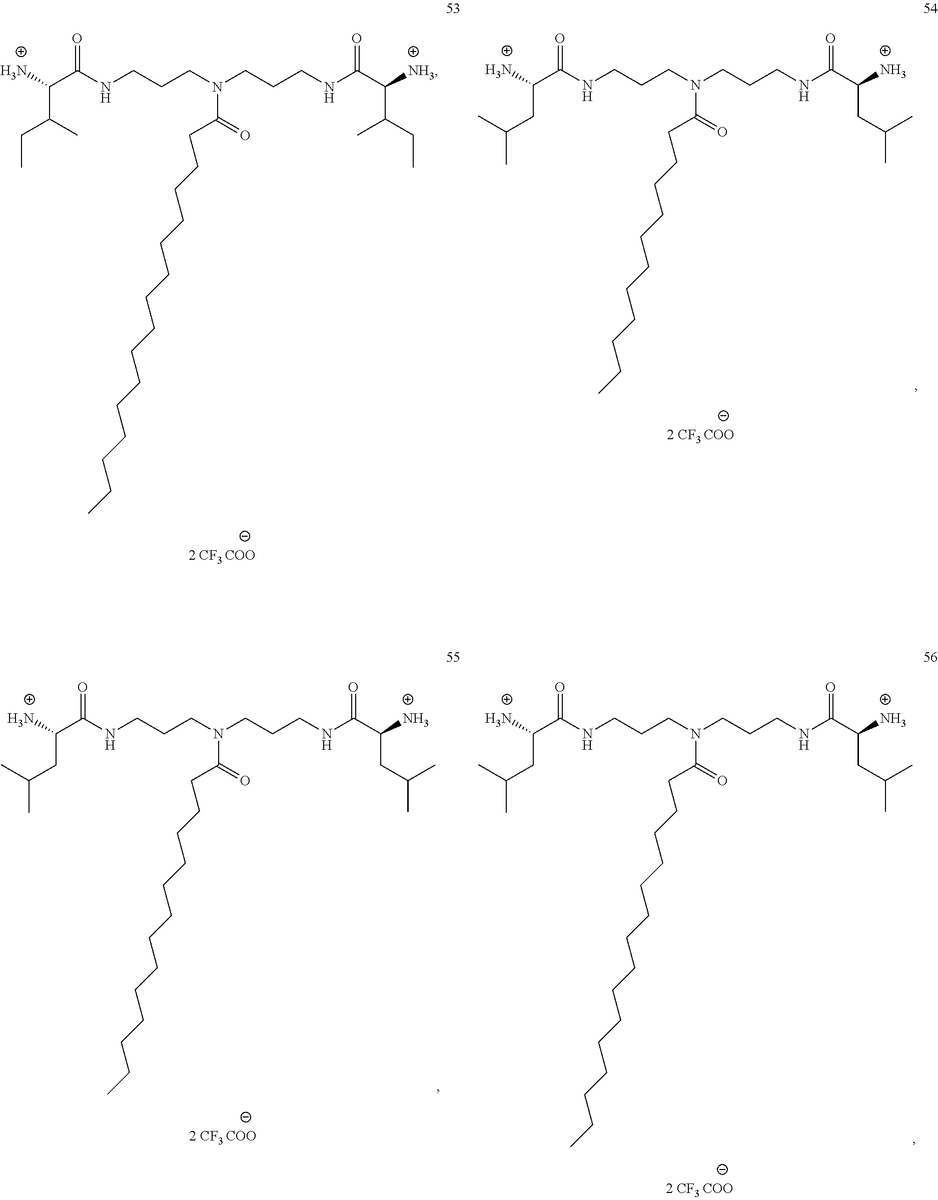

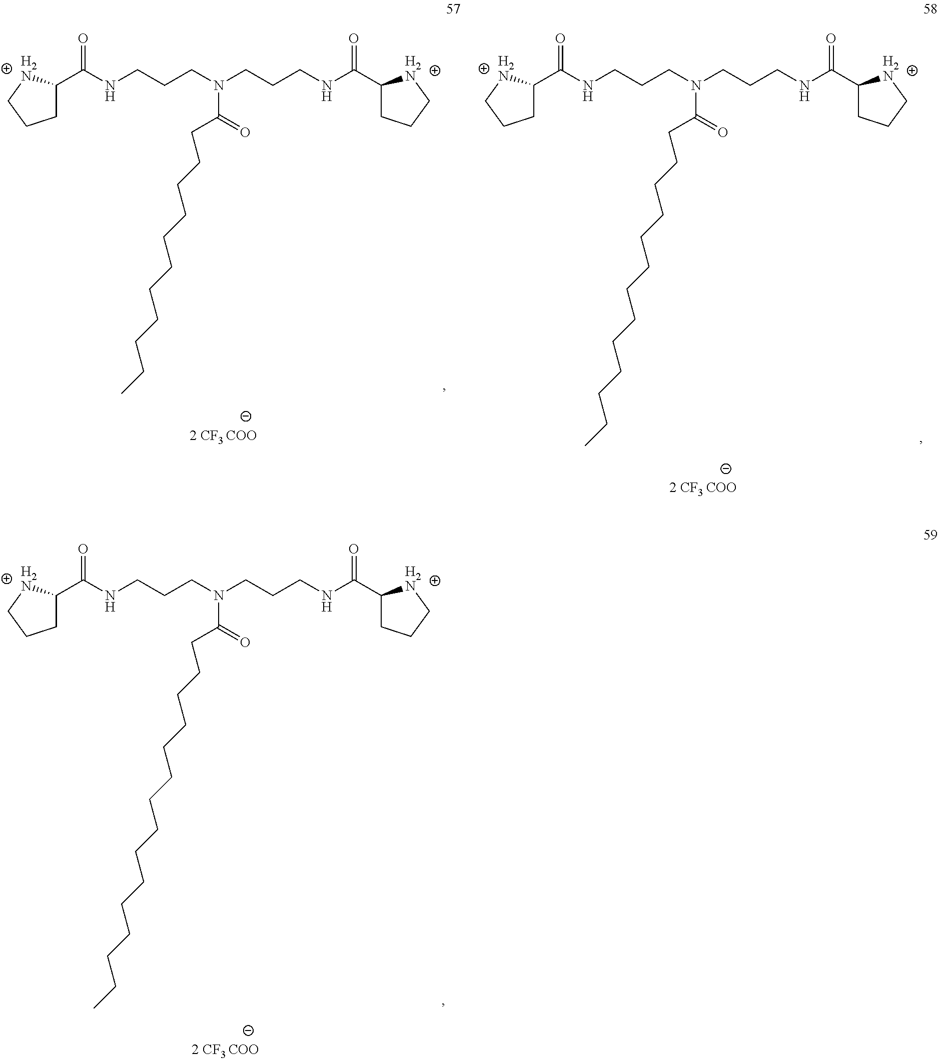

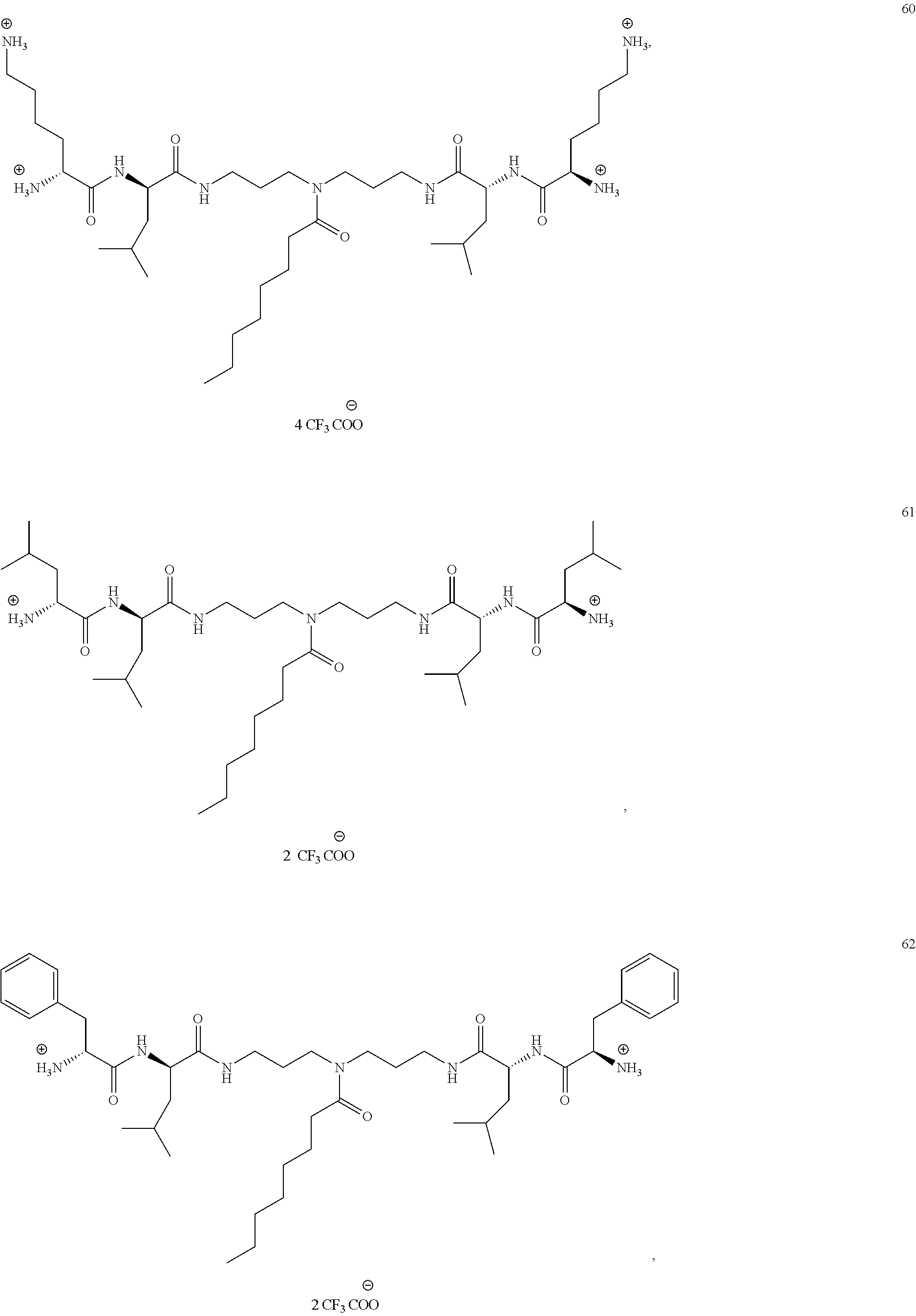

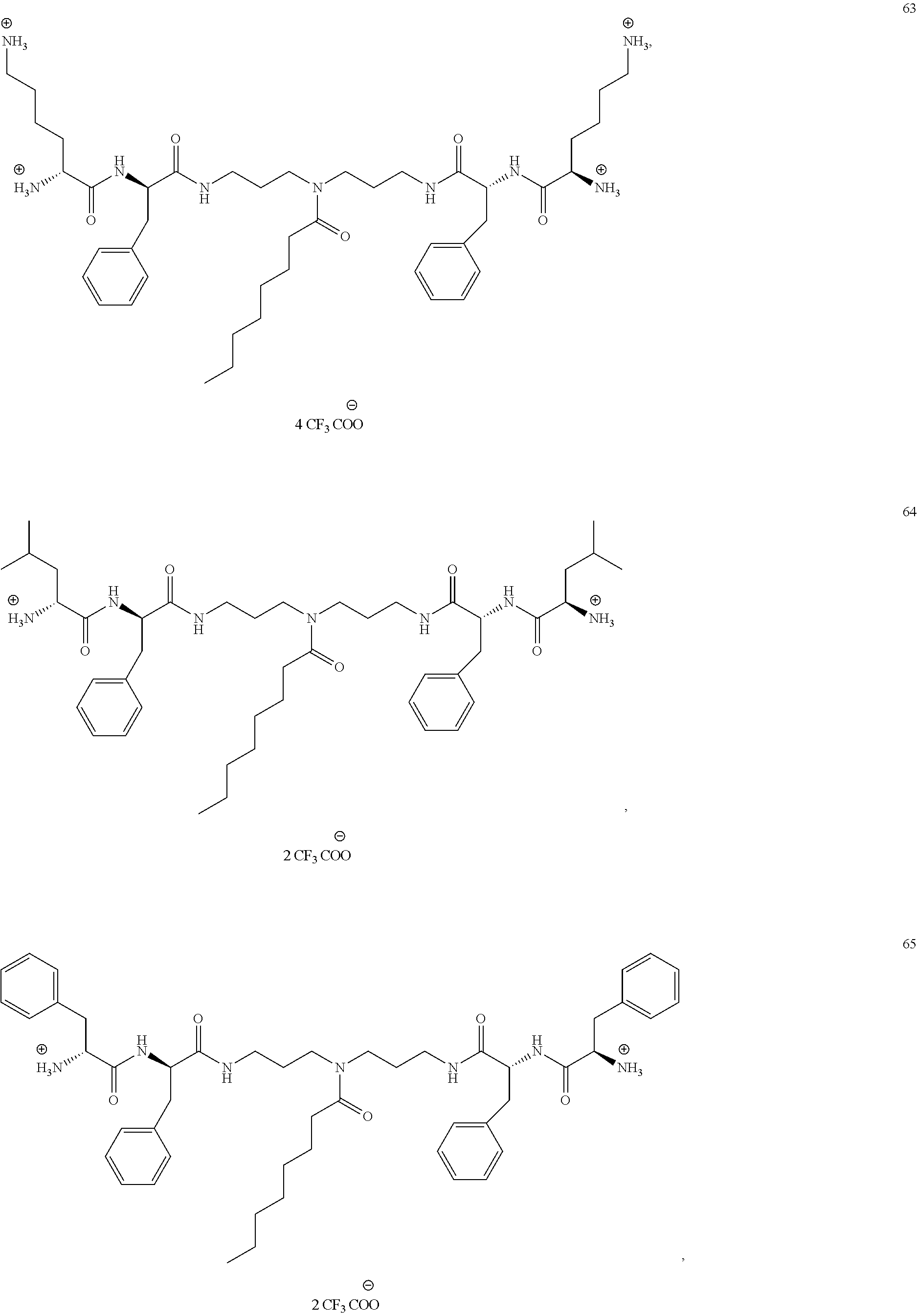

In a preferred embodiment, the compound of formula I is:

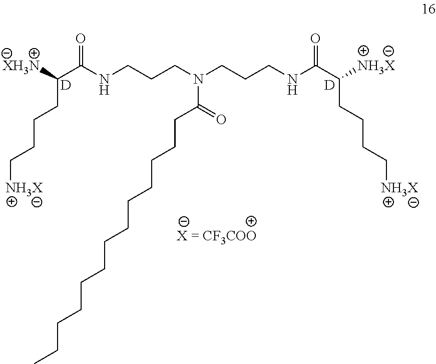

In one embodiment, the compound is selected from the group consisting of compound 2, compound 6, compound 7, compound 11, compound 12, compound 13, compound 15, compound 16 and compound 17.

In one embodiment, the compound is selected from the group consisting of compound 2, compound 6, compound 7, compound 12 and compound 17.

In one embodiment, the compound is selected from the group consisting of compound 2, compound 6, compound 7, compound 12, compound 13, compound 15, compound 16 and compound 17.

In one embodiment, the compound is selected from the group consisting of compound 11, compound 12, compound 15, compound 16 and compound 17.

In one embodiment, the compound is selected from the group consisting of 11 and compound 12.

According to an embodiment, the present disclosure relates to a compound of formula I or its stereoisomers, pharmaceutically acceptable salts, polymorphs, solvates and hydrates thereof, for use in treatment of diseases caused by bacteria, fungi, and virus.

According to another embodiment, the present disclosure relates to a compound of formula I or its stereoisomers, pharmaceutically acceptable salts, polymorphs, solvates, and hydrates thereof, for treating disease or condition caused by Gram-positive and Gram-negative bacteria.

According to another embodiment, the present disclosure relates to a compound of formula I or its stereoisomers, pharmaceutically acceptable salts, polymorphs, solvates and hydrates thereof, for treating disease or condition caused by the drug sensitive bacterium selected from a group consisting of S. aureus, E. faecium and E. coli or any combinations thereof.

According to yet another embodiment, the present disclosure relates to a compound of formula I or its stereoisomers, pharmaceutically acceptable salts, polymorphs, solvates and hydrates thereof, for treating disease or condition caused by the drug resistant bacterium selected from a group consisting of vancomycin-resistant E. faecium, methicillin-resistant S. aureus and β-lactam resistant K. pneumoniae, or combination thereof.

An embodiment of the present disclosure relates to a compound of formula I or its stereoisomers, pharmaceutically acceptable salts, polymorphs, solvates and hydrates thereof, for preparing antimicrobial coatings and/or surfaces with pharmaceutical compositions.

An embodiment of the present disclosure relates to a compound of formula I or its stereoisomers, pharmaceutically acceptable salts, polymorphs, solvates and hydrates thereof, for preparing antimicrobial coatings and/or surfaces without pharmaceutical compositions.

In an embodiment of the present disclosure, it is provided that the surface disclosed is formed from material selected from the group consisting of metals, ceramics, glass, polymers, plastics, fibers and combinations thereof.

According to an embodiment, the present disclosure relates to a pharmaceutical composition comprising a compound of formula (I) or a pharmaceutically acceptable salt thereof together with a pharmaceutically acceptable carrier, optionally in combination with one or more other pharmaceutical compositions.

In an embodiment of the present disclosure, there is provided a process of preparation of compounds of formula (I) or its stereoisomers, pharmaceutically acceptable salts, polymorphs, solvates and hydrates thereof.

### ABBREVIATIONS

The following abbreviations are employed in the examples and elsewhere herein:

### DCM: Dichloromethane,

### NaCl: Sodium Chloride,

Na₂CO₃: Sodium Carbonate,

### DMF: N, N-Dimethylformamide,

### DMSO: N, N-Dimethylsulfoxide,

HBTU: O-Benzotriazole-N, N, N′,N′-tetramethyl-uronium-hexafluorophosphate,

### DIPEA: N, N-Diisopropylethylamine,

HCl: Hydrochloric acid,
RT: Room temperature,

### μM: Micromolar.

The compounds of formula (I) may be prepared as outlined in the Scheme 1-8 given below:

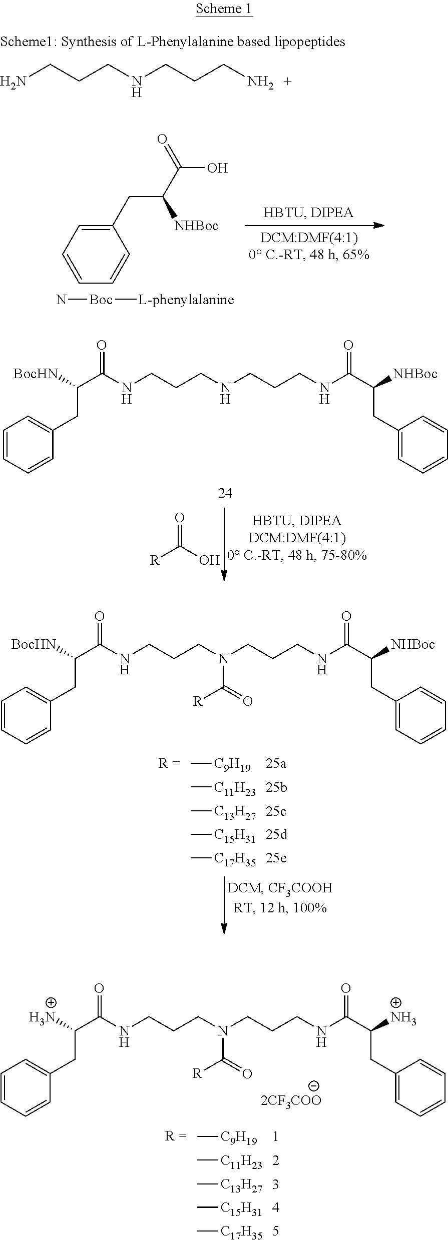

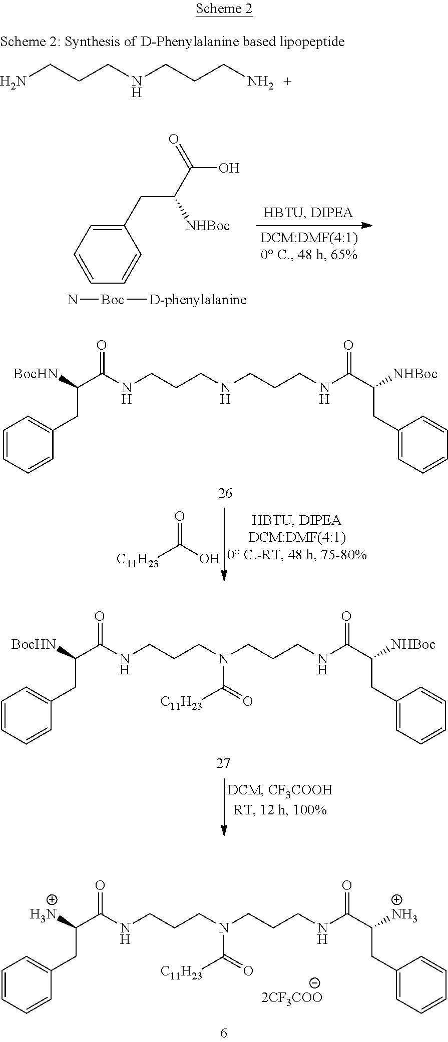

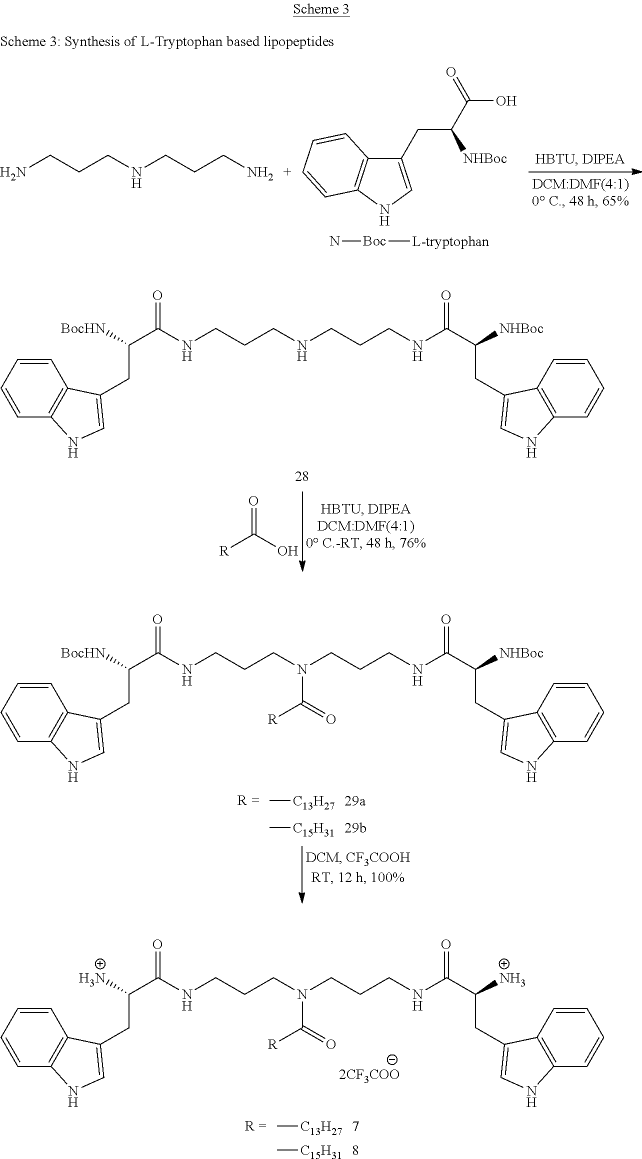

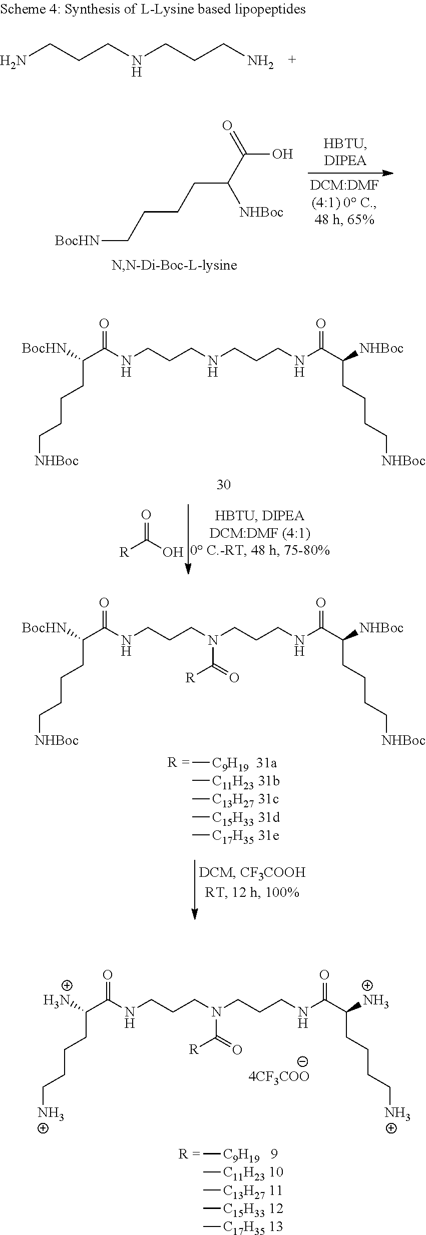

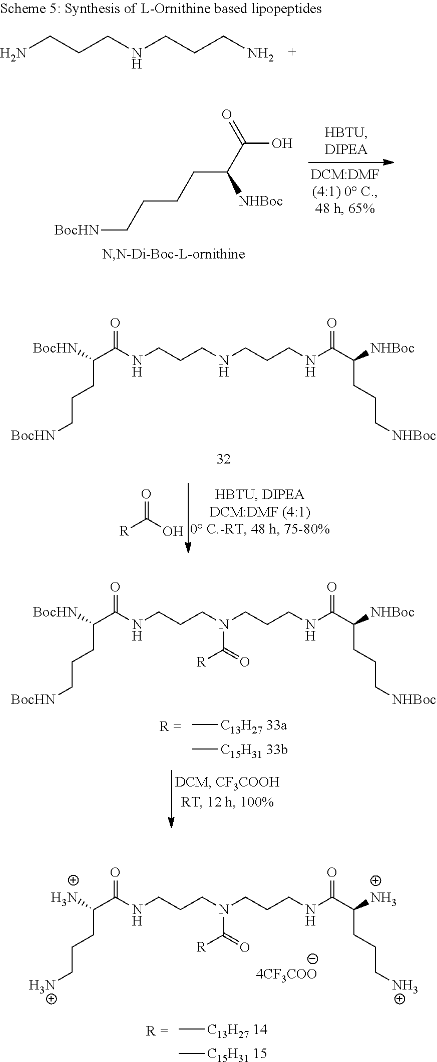

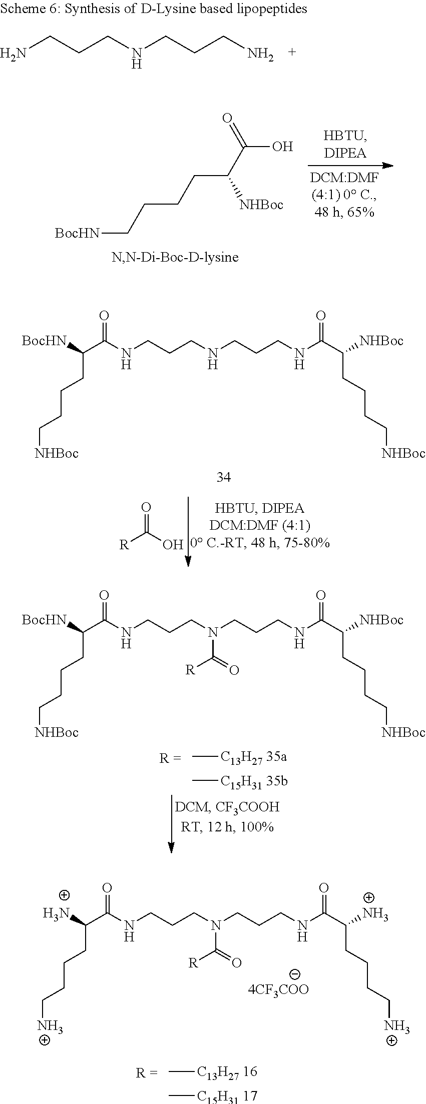

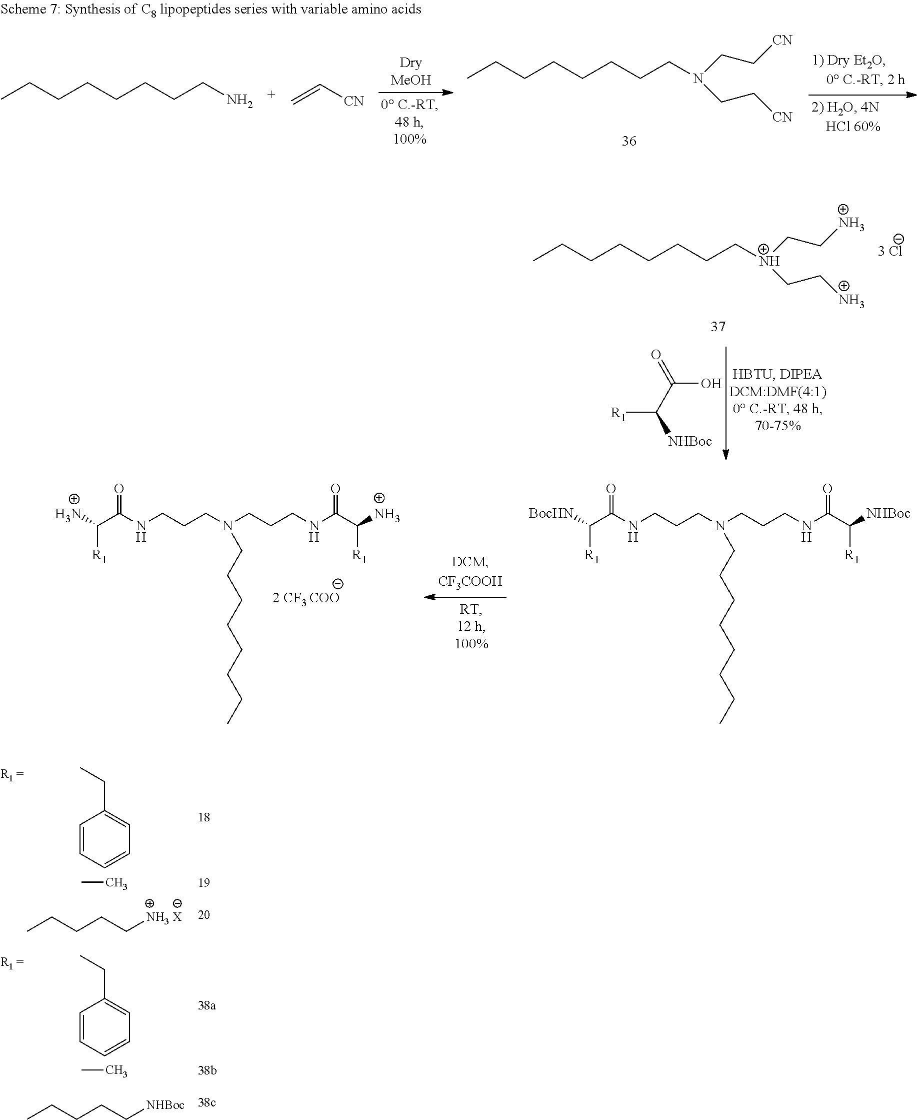

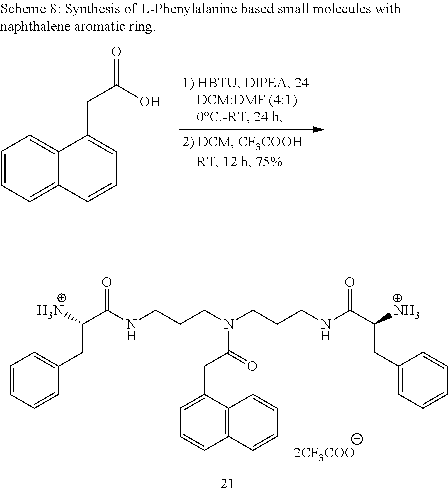 [SMILES: O=C(O)Cc1cccc2ccccc12 ; [NH3+][C@@H](Cc1ccccc1)C(=O)NCCCN(CCCNC(=O)[C@@H]([NH3+])Cc1ccccc1)C(=O)Cc1cccc2ccccc12]

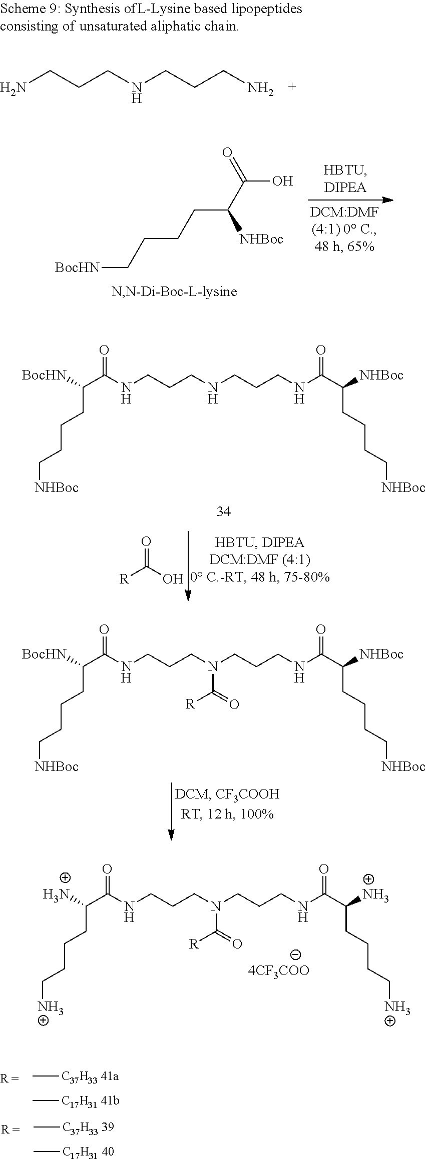

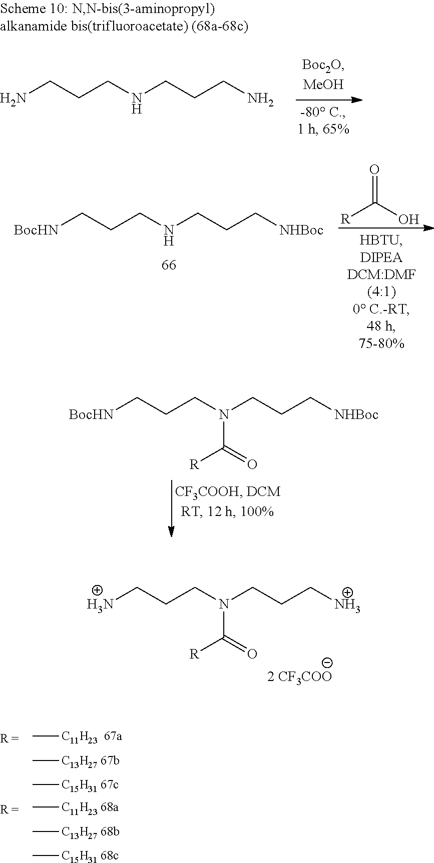

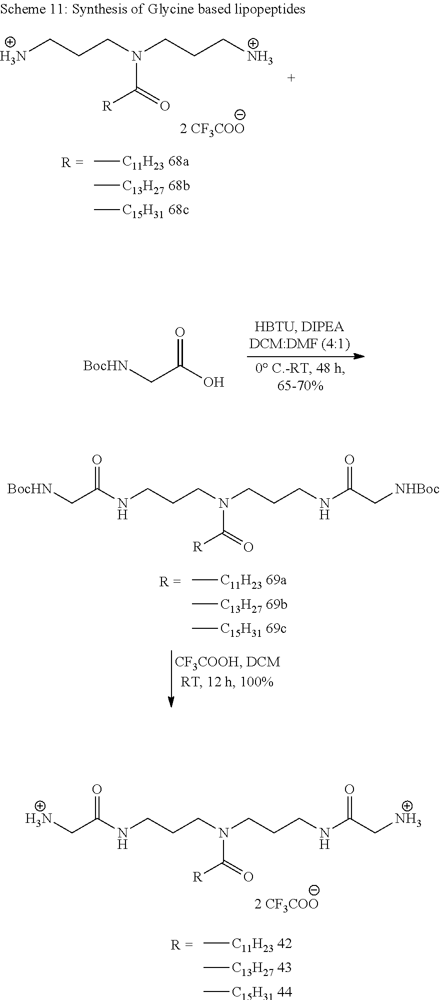

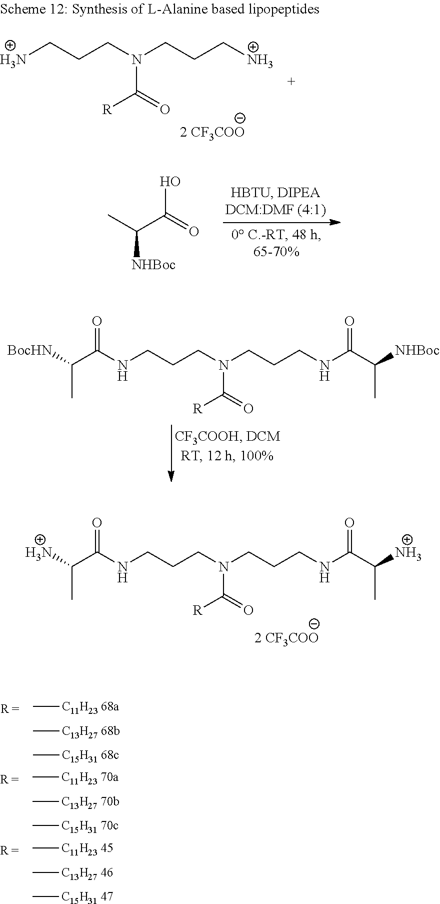

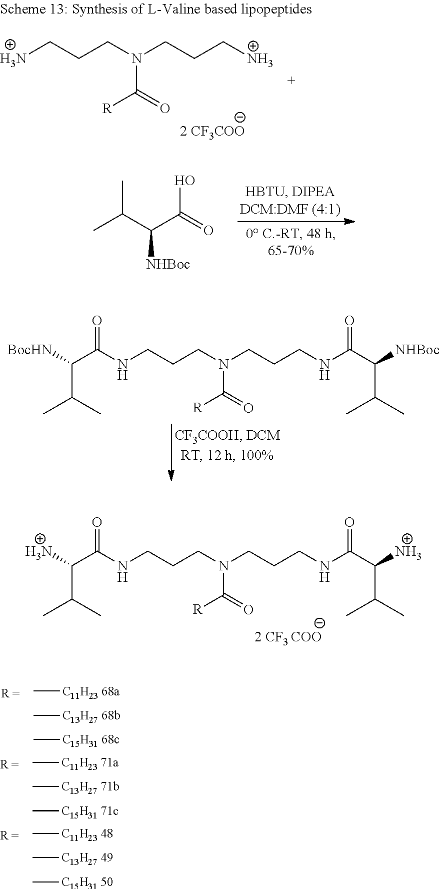

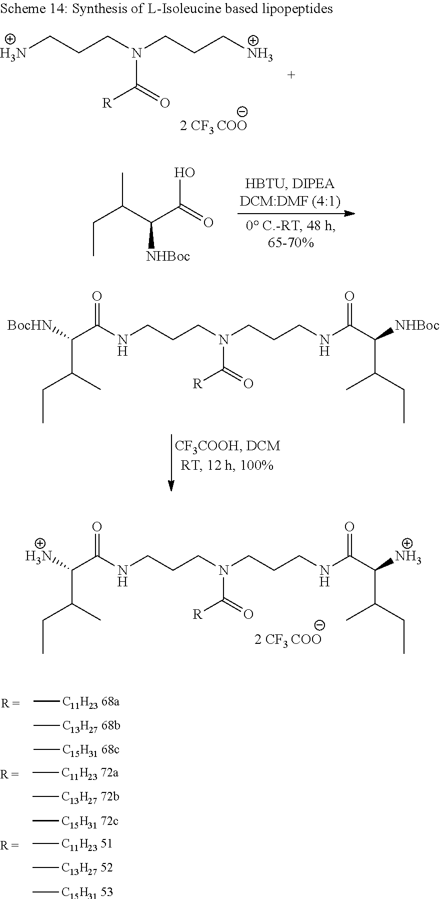

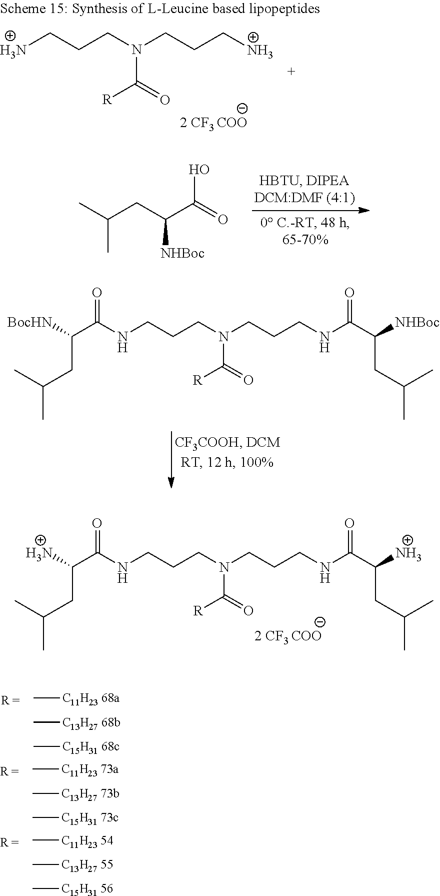

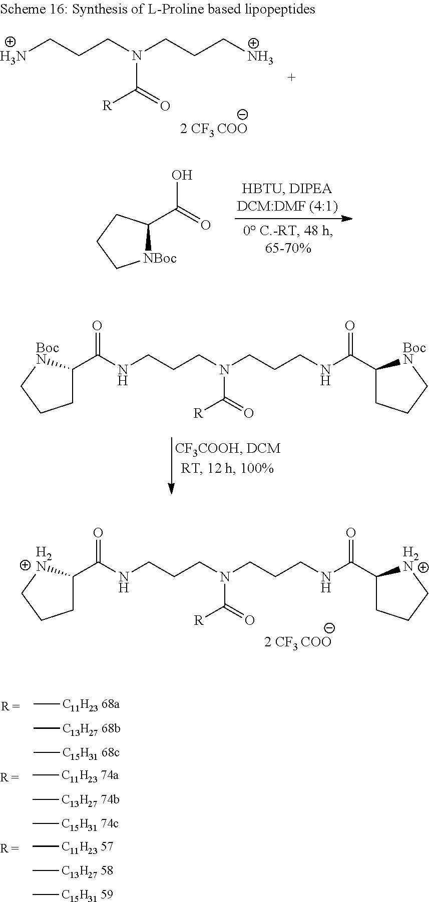

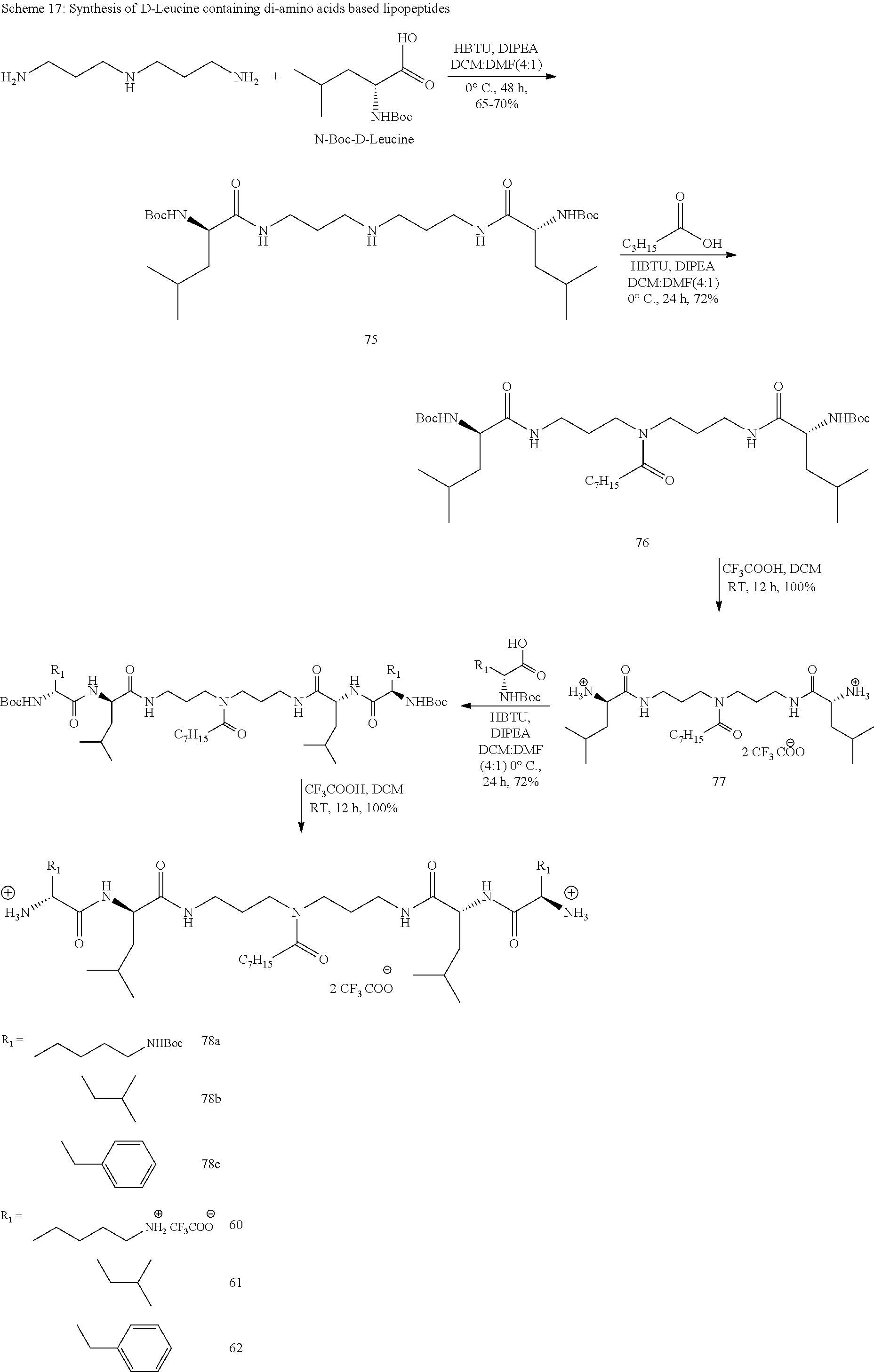

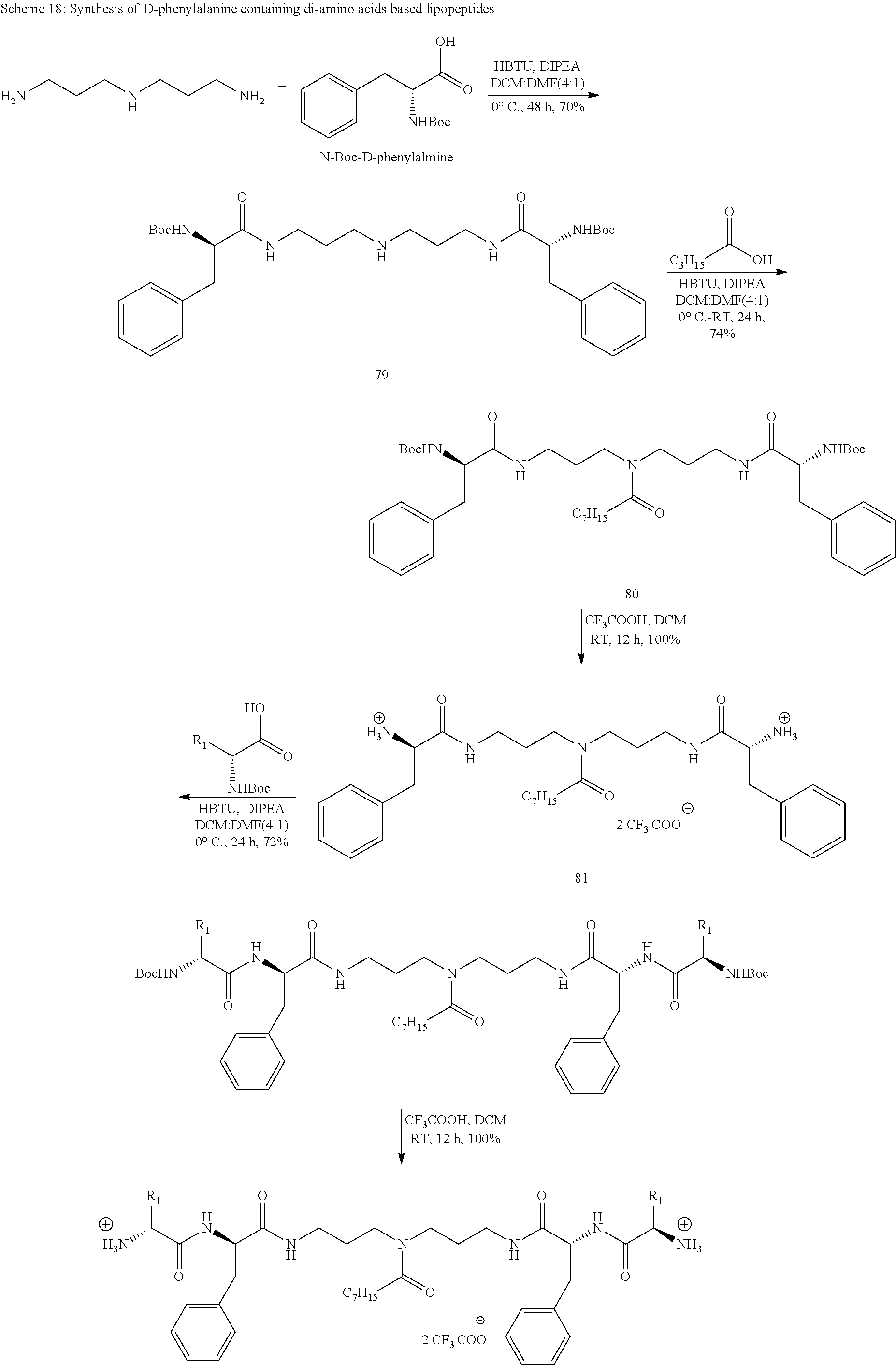

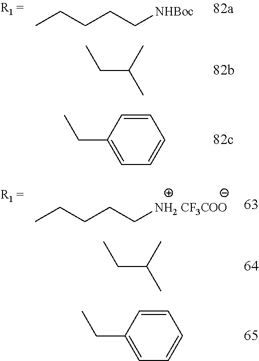

### EXAMPLES

The following examples provide the details about the synthesis, activities, and applications of the compounds of the present invention. It should be understood the following is representative only, and that the invention is not limited by the details set forth in these examples.

### Materials and Methods:

All the solvents were of reagent grade, which were distilled and dried before its uses. All the reagents were purchased from Sigma-Aldrich, Alfa-Aesar, S.D. Fine, Avra and Spectrochem, were used without further purification. Analytical thin layer chromatography (TLC) was performed on E. Merck TLC plates pre-coated with silica gel 60 F₂₅₄ and visualization was carried out using UV light and Iodine. Column chromatography was performed on silica gel (60-120 mesh) using different ratio of chloroform and methanol solvent system. Nuclear magnetic resonance spectra were recorded on Bruker (AV-400) 400 MHz spectrometer in deuterated solvents. Mass spectra were obtained using 6538-UHD Accurate mass Q-TOF LC-MS instrument. Infrared (IR) spectra of the compounds (in Chloroform or Methanol) were recorded on Bruker IFS66 V/s spectrometer using NaCl crystal. For optical density measurement Tecan InfinitePro series M200 Microplate Reader was used. Bacterial strains, S. aureus (MTCC 737) and E. coli (MTCC 443) were purchased from MTCC (Chandigarh, India). MRSA (ATCC 33591), Enterococcus faecium (ATCC 19634), vancomycin resistant Enterococcus faecium (VRE) (ATCC 51559) and Klebsiella pneumoniae (ATCC 700603) were obtained from ATCC (Rockville, Md., USA).

### Microorganisms and Culture Conditions:

The antibacterial activity of the synthesized compounds was evaluated against both Gram-negative (E. coli, K. pneumonie) and Gram-positive (S. aureus, MRSA, E. faecium and VRE) bacteria. E. coli was cultured in Luria Bertani broth and S. aureus, MRSA were grown in Yeast-dextrose broth (1 g of beef extract, 2 g of yeast extract, and 5 g of peptone and 5 g of NaCl in 1000 mL of sterile distilled water). Enterococcus faecium and VRE were grown in Brain Heart Infusion broth (BHI). The bacterial samples were freeze dried and stored at −80° C. 5 μL of these stocks were added to 3 mL of the respective media and the culture was grown for 6 h at 37° C. prior to the experiments.

### Example 1

Amino acid based lapidated small molecules (1-23, 39, 40 and 42-65) of the instant disclosure were synthesized from N-Boc protected aminoacids, norspermidine and fatty acids, through simple amide coupling reaction using O-Benzotriazole-N,N,N′,N′-tetramethyl-uronium-hexafluorophos phate (HBTU) as coupling agent followed by deprotection using trifluoroacetic acid. The steps employed in the method of synthesizing the compounds (1-23, 39, 40 and 42-65) are further elaborated below in Examples 1.1-1.20.

### Example 1.1: Synthesis of N¹-(Boc-^{L}Phe)-N³-[{3-(Boc-^{L}Phe)amino}propyl]propane-1,3-diamine (24)

About 5 g (2 equivalents, 18.85 mmol) of N-Boc-L-Phenylalanine was dissolved in about 25 mL of dry DCM at 0° C. In the reaction mixture about 9.8 mL (6 equivalents, 56.55 mmol) of DIPEA was added followed by about 7.2 g (2 equivalents, 18.85 mmol) of HBTU. Now about 8 mL of DMF was added to the reaction mixture. After 10 minutes, about 1.2 g (1 equivalent, 9.43 mmol) of norspermidine [Bis (3-aminopropyl) amine] was added drop wise. The reaction mixture was allowed to stir for 48 h at 0° C. Then reaction solvent was evaporated and residue was diluted in ethyl acetate. Thereafter work-up was done with 1N HCl (50 mL, 3 times) followed by saturated Na₂CO₃ solution (50 mL, 3 times). The crude product was collected in ethyl acetate layer. Finally column was done to isolate the product with 65% yield. FT-IR (NaCl): 3311 cm⁻¹ (—NH— str.), 3033 cm⁻¹ (aromatic C—H str.), 2929 cm⁻¹ (—CH₂— asym. str.), 2854 cm⁻¹ (—CH₂— sym. str.), 1700 cm⁻¹ and 1684 cm⁻¹ (C═O str.), 1654 cm⁻¹, 1635 cm⁻¹ (aromatic C═C str.). ¹H-NMR (400 MHz, CDCl₃) δ/ppm: 7.300-7.204 (m, 10H), 7.194 (s, 2H), 5.368 (s, 2H), 4.314-4.298 (d, 2H), 3.370-2.985 (m, 12H), 2.570 (s, 1H), 1.687-1.676 (m, 4H), 1.386 (s, 18H). HRMS (m/z): 626.3891 [(M+H)⁺] (Observed), 626.3918 [(M+H)⁺] (Calculated).

### Example 1.1.1: N,N-bis-[3-(Boc-^{L}Phe)aminopropyl]alkanamide (25a-25e)

About 1.5 equivalents of saturated aliphatic acid (C10, C12, C14, C16, and C18) were dissolved in dry DCM at 0° C. In the reaction mixture about 4 equivalents of DIPEA was added followed by about 1.5 equivalents of HBTU. Now DMF was added to the reaction mixture. After 10 minutes, about 1 equivalent of 24 was added drop wise dissolving it in dry DCM. The reaction mixture was brought to RT and allowed to stir for 24 h. Then reaction solvent was evaporated and residue was diluted in ethyl acetate. Thereafter work-up was done at first with 1N HCl (3 times) followed by saturated Na₂CO₃ solution (3 times). The crude product was collected in ethyl acetate layer. Finally column was done to isolate the puree product of 75-80% yield.

### Example 1.1.1.1: N,N-bis-[{3-(Boc-Phe)amino}propyl]decanamide (25a)

FT-IR (NaCl): 3304 cm⁻¹ (—NH— str.), 3029 cm⁻¹ (aromatic C—H str.), 2927 cm⁻¹ (—CH₂— asym. str.), 2855 cm⁻¹ (—CH₂— sym. str.), 1699 cm⁻¹ and 1684 cm⁻¹ (C═O str.), 1652 cm⁻¹, 1635 cm⁻¹ (aromatic C═C str.). ¹H-NMR (400 MHz, CDCl₃) δ/ppm: 7.246-7.097 (m, 12H), 5.350-5.229 (m, 2H), 4.355-4.315 (d, 2H), 3.384-2.871 (m, 12H), 2.215-2.195 (t, 2H), 1.597-1.463 (m, 6H), 1.353 (s, 18H), 1.255 (s, 12H), 0.889-0.855 (t, 3H). HRMS (m/z): 780.5268 [(M+H)⁺] (Observed), 780.5275 [(M+H)⁺] (Calculated).

### Example 1.1.1.2: N,N-bis-[{3-(Boc-^{L}Phe)amino}propyl]dodecanamide (25b)

FT-IR (NaCl): 3300 cm⁻¹ (—NH— str.), 3030 cm⁻¹ (aromatic C—H str.), 2928 cm⁻¹ (—CH₂— asym. str.), 2854 cm⁻¹ (—CH₂— sym. str.), 1699 cm⁻¹ and 1683 cm⁻¹ (C═O str.), 1652 cm⁻¹, 1635 cm¹ (aromatic C═C str.). ¹H-NMR (400 MHz, CDCl₃) δ/ppm: 7.215-7.096 (m, 12H), 5.417-5.277 (m, 2H), 4.360-4.328 (d, 2H), 3.356-2.869 (m, 12H), 2.209-2.189 (t, 2H), 1.714-1.463 (m, 6H), 1.351 (s, 18H), 1.244 (s, 16H), 0.885-0.851 (t, 3H). HRMS (m/z): 808.5593 [(M+H)⁺] (Observed), 808.5588 [(M+H)⁺] (Calculated).

### Example 1.1.1.3: N,N-bis-[{3-(Boc-^{L}Phe)amino}propyl]tetradecanamide (25c)

FT-IR (NaCl): 3301 cm¹ (—NH— str.), 3028 cm⁻¹ (aromatic C—H str.), 2926 cm⁻¹ (—CH₂— asym. str.), 2855 cm⁻¹ (—CH₂— sym. str.), 1699 cm⁻¹ and 1684 cm⁻¹ (C═O str.), 1654 cm⁻¹, 1637 cm⁻¹ (aromatic C═C str.). ¹H-NMR (400 MHz, CDCl₃) δ/ppm: 7.215-7.100 (m, 12H), 5.495-5.345 (m, 2H), 4.352-4.333 (d, 2H), 3.451-2.861 (m, 12H), 2.202-2.182 (t, 2H), 1.644-1.547 (m, 6H), 1.345 (s, 18H), 1.235 (s, 20H), 0.879-0.845 (t, 3H). HRMS (m/z): 836.5908 [(M+H)⁺] (Observed), 836.5901 [(M+H)⁺] (Calculated).

### Example 1.1.1.4: N,N-bis-[{3-(Boc-^{L}Phe)amino}propyl]hexadecanamide (25d)

FT-IR (NaCl): 3299 cm⁻¹ (—NH— str.), 3031 cm⁻¹ (aromatic C—H str.), 2927 cm⁻¹ (—CH₂— asym. str.), 2854 cm⁻¹ (—CH₂— sym. str.), 1699 cm⁻¹ and 1684 cm⁻¹ (C═O str.), 1656 cm⁻¹, 1635 cm⁻¹ (aromatic C═C str.). ¹H-NMR (400 MHz, CDCl₃) δ/ppm: 7.211-7.097 (m, 12H), 5.515-5.357 (m, 2H), 4.356-4.337 (d, 2H), 3.425-2.861 (m, 12H), 2.200-2.181 (t, 2H), 1.690-1.435 (m, 6H), 1.343 (s, 18H), 1.233 (s, 24H), 0.877-0.843 (t, 3H).). HRMS (m/z): 864.6227 [(M+H)⁺] (Observed), 864.6214 [(M+H)⁺] (Calculated).

### Example 1.1.1.5: N,N-bis-[{3-(Boc-^{L}Phe)amino}propyl]octadecanamide (25e)

FT-IR (NaCl): 3302 cm⁻¹ (—NH— str.), 3028 cm⁻¹ (aromatic C—H str.), 2927 cm⁻¹ (—CH₂— asym. str.), 2855 cm⁻¹ (—CH₂— sym. str.), 1699 cm⁻¹ and 1684 cm⁻¹ (C═O str.), 1652 cm⁻¹, 1635 cm⁻¹ (aromatic C═C str.). ¹H-NMR (400 MHz, CDCl₃) δ/ppm: 7.245-7.100 (m, 12H), 5.338-5.216 (m, 2H), 4.338-4.319 (d, 2H), 3.484-2.802 (m, 12H), 2.214-2.194 (t, 2H), 1.631-1.3578 (m, 6H), 1.354 (s, 18H), 1.250 (s, 28H), 0.894-0.860 (t, 3H). HRMS (m/z): 892.6535 [(M+H)⁺] (Observed), 892.6527 [(M+H)⁺] (Calculated).

### Example 1.1.2: N,N-bis-[{3-(^{L}Phe)amino}propyl]alkanamide bis(trifluoroacetate) (1-5)

At first 1 equivalent of 25a-25e was dissolved in DCM. To the intensely stirred solution 4 equivalents (excess amount) of trifluoroacetic acid (TFA) was added and allowed to stir at RT for 12 h. After that reaction solvent and unused TFA was removed to get the pure product with 100% yield.

### Example 1.1.2.1: N,N-bis-[{3-(^{L}Phe)amino}propyl]decanamide bis(trifluoroacetate) (1)

FT-IR (NaCl): 3270 cm⁻¹ (—NH— str.), 3034 cm⁻¹ (aromatic C—H str.), 2930 cm⁻¹ (—CH₂— asym. str.), 2859 cm⁻¹ (—CH₂— sym. str.), 1673 cm⁻¹ (C═O str.), 1618 cm⁻¹, 1577 cm⁻¹ (aromatic C═C str.). ¹H-NMR (400 MHz, DMSO-d₆) δ/ppm: 8.473-8.231 (m, 8H), 7.339-7.219 (m, 10H), 3.939 (s, 2H), 3.187-2.889 (m, 12H), 2.200-2.165 (t, 2H), 1.527-1.446 (m, 6H), 1.221 (s, 12H), 0.864-0.830 (t, 3H). HRMS (m/z): 580.4225 [(M+H)⁺](Observed), 580.4243 [(M+H)⁺] (Calculated).

### Example 1.1.2.2: N,N-bis-[{3-(^{L}Phe)amino}propyl]dodecanamide bis(trifluoroacetate) (2)

FT-IR (NaCl): 3275 cm⁻¹ (—NH— str.), 3034 cm⁻¹ (aromatic C—H str.), 2927 cm⁻¹ (—CH₂— asym. str.), 2855 cm⁻¹ (—CH₂— sym. str.), 1680 cm⁻¹ (C═O str.), 1620 cm⁻¹, 1576 cm⁻¹ (aromatic C═C str.). ¹H-NMR (400 MHz, DMSO-d₆) δ/ppm: 8.479-8.235 (m, 8H), 7.341-7.218 (m, 10H), 3.940 (s, 2H), 3.171-2.924 (m, 12H), 2.199-2.165 (t, 2H), 1.526-1.444 (m, 6H), 1.221 (s, 16H), 0.865-0.831 (t, 3H). HRMS (m/z): 608.4510 [(M+H)⁺] (Observed), 608.4540 [(M+H)⁺] (Calculated).

### Example 1.1.2.3: N,N-bis-[{3-(^{L}Phe)amino}propyl]tetradecanamide bis(trifluoroacetate) (3)

FT-IR (NaCl): 3275 cm⁻¹ (—NH— str.), 3033 cm⁻¹ (aromatic C—H str.), 2927 cm⁻¹ (—CH₂— asym. str.), 2856 cm⁻¹ (—CH₂— sym. str.), 1679 cm⁻¹ (C═O str.), 1620 cm⁻¹, 1576 cm⁻¹ (aromatic C═C str.). ¹H-NMR (400 MHz, DMSO-d₆) δ/ppm: 8.506-8.257 (m, 8H), 7.333-7.218 (m, 10H), 3.944 (s, 2H), 3.184-2.905 (m, 12H), 2.197-2.163 (t, 2H), 1.525-1.443 (m, 6H), 1.222 (s, 20H), 0.865-0.831 (t, 3H). HRMS (m/z): 636.4843 [(M+H)⁺] (Observed), 636.4853 [(M+H)⁺] (Calculated).

### Example 1.1.2.4: N,N-bis-[{3-(^{L}Phe)amino}propyl]hexadecanamide bis(trifluoroacetate) (4)

FT-IR (NaCl): 3277 cm⁻¹ (—NH— str.), 3034 cm⁻¹ (aromatic C—H str.), 2928 cm⁻¹ (—CH₂— asym. str.), 2857 cm⁻¹ (—CH₂— sym. str.), 1676 cm⁻¹ (C═O str.), 1618 cm⁻¹, 1577 cm⁻¹ (aromatic C═C str.). ¹H-NMR (400 MHz, DMSO-d₆) δ/ppm: 8.500-8.254 (m, 8H), 7.334-7.218 (m, 10H), 3.944 (s, 2H), 3.202-2.891 (m, 12H), 2.197-2.163 (t, 3H), 1.525-1.444 (m, 6H), 1.226 (s, 24H), 0.866-0.832 (t, 3H). HRMS (m/z): 664.5156 [(M+H)⁺] (Observed), 664.5166 [(M+H)⁺] (Calculated).

### Example 1.1.2.5: N,N-bis-[{3-(^{L}Phe)amino}propyl]octadecanamide bis(trifluoroacetate) (5)

FT-IR (NaCl): 3275 cm⁻¹ (—NH— str.), 3034 cm⁻¹ (aromatic C—H str.), 2926 cm⁻¹ (—CH₂— asym. str.), 2854 cm⁻¹ (—CH₂— sym. str.), 1679 cm⁻¹ (C═O str.), 1619 cm⁻¹, 1576 cm⁻¹ (aromatic C═C str.). ¹H-NMR (400 MHz, DMSO-d₆) δ/ppm: 8.500-8.253 (m, 8H), 7.334-7.217 (m, 10H), 3.943 (s, 2H), 3.201-2.891 (m, 12H), 2.196-2.163 (t, 2H), 1.524-1.443 (m, 6H), 1.226 (s, 28H), 0.866-0.832 (t, 3H). HRMS (m/z): 692.5433 [(M+H)⁺] (Observed), 692.5479 [(M+H)⁺] (Calculated).

### Example 1.2: Synthesis of N¹-(Boc-^{D}Phe)-N³-[{3-(Boc-^{D}Phe)amino}propyl]propane-1,3-diamine (26)

About 2.3 g (2 equivalents, 8.67 mmol) of N-Boc-D-Phenylalanine was dissolved in about 10 mL of dry DCM at 0° C. Then to the reaction mixture about 4.5 mL (6 equivalents, 26.01 mmol) of DIPEA was added followed by about 3.3 g (2 equivalents, 8.67 mmol) of HBTU. Now about 3 mL of DMF was added to the reaction mixture. After 10 minutes, about 0.57 g (1 equivalent, 4.3 mmol) of norspermidine [Bis (3-aminopropyl) amine] was added drop wise. The reaction mixture was allowed to stir for 48 h at 0° C. Then reaction solvent was evaporated and residue was diluted in ethyl acetate. Thereafter work-up was done at first with 1N HCl (25 mL, 3 times) followed by saturated Na₂CO₃ solution (25 mL, 3 times). The crude product was collected in ethyl acetate layer. Finally column was done to isolate the product with 66% yield. FT-IR (NaCl): 3303 cm⁻¹ (—NH— str.), 3015 cm⁻¹ (aromatic C—H str.), 2929 cm⁻¹ (—CH₂— asym. str.), 2853 cm⁻¹ (—CH₂— sym. str.), 1699 cm⁻¹ and (C═O str.), 1654 cm⁻¹, 1636 cm⁻¹ (aromatic C═C str.). ¹H-NMR (400 MHz, CDCl₃) δ/ppm: 7.245-7.192 (m, 12H), 5.415 (s, 2H), 4.334-4.328 (d, 2H), 3.377-2.995 (m, 12H), 2.568 (s, 1H), 1.698-1.654 (m, 4H), 1.375 (s, 18H). HRMS (m/z): 626.3901 [(M+H)⁺] (Observed), 626.3918 [(M+H)+](Calculated).

### Example 1.2.1: Synthesis of N,N-bis-[{3-(Boc-^{D}Phe)amino}propyl]dodecanamide (27)

About 0.24 g (1.5 equivalents, 1.2 mmol) of dodecanoic acid was dissolved in 3 mL of dry DCM at 0° C. In the reaction mixture about 0.3 mL (4 equivalents, 3.2 mmol) of DIPEA was added followed by about 0.46 g (1.5 equivalents, 1.2 mmol) of HBTU. Now about 1 mL of DMF was added to the reaction mixture. After 10 minutes, about 0.5 g (1 equivalent, 0.8 mmol) of 26 was added drop wise dissolving it in 1 mL of dry DCM. Then the reaction mixture was brought to RT and allowed to stir for 24 h. Then reaction solvent was evaporated and residue was diluted in ethyl acetate. Thereafter work-up was done at first with 1N HCl (15 mL, 3 times) followed by saturated Na₂CO₃ solution (15 mL, 3 times). The crude product was collected in ethyl acetate layer. Finally column was done to isolate the pure product with 77% yield. FT-IR (NaCl): 3303 cm⁻¹ (—NH— str.), 3034 cm⁻¹ (aromatic C—H str.), 2926 cm⁻¹ (—CH₂— asym. str.), 2854 cm⁻¹ (—CH₂— sym. str.), 1697 cm⁻¹ and 1687 cm⁻¹ (C═O str.), 1654 cm⁻¹, 1632 cm⁻¹ (aromatic C═C str.). ¹H-NMR (400 MHz, CDCl₃) δ/ppm: 7.212-7.094 (m, 12H), 5.55-5.442 (m, 2H), 4.353-4.334 (d, 2H), 3.452-2.892 (m, 12H), 2.205-2.185 (t, 2H), 1.647-1.451 (m, 6H), 1.434 (s, 18H), 1.343 (s, 16H), 0.878-0.844 (t, 3H). HRMS (m/z): 808.5590 [(M+H)⁺] (Observed), 808.5588 [(M+H)⁺] (Calculated).

### Example 1.2.2: Synthesis of N,N-bis-[{3-(^{D}Phe)amino}propyl]dodecanamide bis(trifluoroacetate) (6)

At first 1 equivalent of 27 was dissolved in DCM. To the stirred solution 4 equivalents (excess amount) of trifluoroacetic acid (TFA) was added and allowed to stir at RT for 12 h. After that reaction solvent and unused TFA was removed to get the pure product of 100% yield. FT-IR (NaCl): 3305 cm⁻¹ (—NH— str.), 3030 cm⁻¹ (aromatic C—H str.), 2929 cm⁻¹ (—CH₂— asym. str.), 2852 cm⁻¹ (—CH₂— sym. str.), 1680 cm⁻¹ (C═O str.), 1620 cm⁻¹, 1576 cm⁻¹ (aromatic C═C str.). ¹H-NMR (400 MHz, DMSO-d₆) δ/ppm: 8.483-8.240 (m, 8H), 7.345-7.222 (m, 10H), 3.945 (s, 2H), 3.191-2.893 (m, 12H), 2.203-2.169 (t, 2H), 1.531-1.449 (m, 6H), 1.225 (s, 16H), 0.870-0.836 (t, 3H). HRMS (m/z): 608.4527 [(M+H)⁺] (Observed), 608.4540 [(M+H)⁺] (Calculated).

### Example 1.3: Synthesis of N¹-(Boc-^{L}Trp)-N³-[{3-(Boc-^{L}Trp)amino}propyl]propane-1,3-diamine (28)

About 4 g (2 equivalents, 13.14 mmol) of N-Boc-L-Tryptophan was dissolved in about 20 mL of dry DCM at 0° C. In the reaction mixture about 7.5 mL (6 equivalents, 39.4 mmol) of DIPEA was added followed by about 4.99 g (2 equivalents, 13.143 mmol) of HBTU. Now about 5 mL of DMF was added to the reaction mixture. After 10 minutes, about 0.86 g (1 equivalent, 6.6 mmol) of norspermidine [Bis (3-aminopropyl) amine] was added to the reaction mixture drop wise. The reaction mixture was allowed to stir for 48 h at 0° C. Then reaction solvent was evaporated and residue was diluted in ethyl acetate. Thereafter work-up was done at first with 1N HCl (50 mL, 3 times) followed by saturated Na₂CO₃ solution (50 mL, 3 times). The crude product was collected in ethyl acetate layer. Finally column was done to isolate the product with 66% yield. FT-IR (NaCl): 3313 cm⁻¹ (—NH— str.), 2957 cm⁻¹ (aromatic C—H str.), 2920 cm⁻¹ (—CH₂— asym. str.), 2848 cm⁻¹ (—CH₂— sym. str.), 1718 cm⁻¹ (C═O str.), 1622 cm⁻¹, 1541 cm⁻¹ (aromatic C═C str.). ¹H-NMR (400 MHz, CDCl₃) δ/ppm: 9.054 (s, 2H), 7.620-6.990 (m, 10H), 6.609 (m, 2H), 5.480 (s, 2H), 4.309 (s, 2H), 3.349-2.997 (m, 12H), 2.055 (s, 1H), 1.638-1.529 (m, 4H), 1.482 (s, 18H). HRMS (m/z): 704.4128 [(M+H)⁺] (Observed), 704.4136 [(M+H)⁺] (Calculated).

### Example 1.3.1: Synthesis of N,N-bis-[{3-(Boc-^{L}Trp)amino}propyl]alkanamide (29a-29b)

About 1.5 equivalents of saturated aliphatic acid (C14, C16) were dissolved in dry DCM at 0° C. In the reaction mixture about 4 equivalents of DIPEA was added followed by about 1.5 equivalents of HBTU. Now DMF was added to the reaction mixture. After 10 minutes, about 1 equivalent of 28 was added drop wise dissolving it in dry DCM. The reaction mixture was brought to RT and allowed to stir for 24 h. Then reaction solvent was evaporated and residue was diluted in ethyl acetate. Thereafter work-up was done at first with 1N HCl (3 times) followed by saturated Na₂CO₃ solution (3 times). The crude product was collected in ethyl acetate layer. Finally column was done to isolate the pure product of 75-80% yield.

### Example 1.3.1.1: N,N-bis-[{3-(Boc-^{L}Trp)amino}propyl]tetradecanamide (29a)

FT-IR (NaCl): 3311 cm⁻¹ (—NH— str.), 2977 cm⁻¹ (aromatic C—H str.), 2926 cm⁻¹ (—CH₂-asym. str.), 2853 cm⁻¹ (—CH₂— sym. str.), 1695 cm⁻¹ (C═O str.), 1658 cm⁻¹, 1630 cm⁻¹ (aromatic C═C str.). ¹H-NMR (400 MHz, CDCl₃) δ/ppm: 8.490-8.255 (d, 2H), 7.518-6.605 (m, 12H), 5.269-5.212 (m, 2H), 4.425-4.407 (d, 2H), 3.297-2.837 (m, 12H), 2.111-2.076 (t, 2H), 1.527-1.450 (m, 6H), 1.370 (s, 18H), 1.251 (s, 20H), 0.894-0.861 (t, 3H). HRMS (m/z): 914.6107 [(M+H)⁺] (Observed), 914.6119 [(M+H)⁺] (Calculated).

### Example 1.3.1.2: N,N-bis-[{3-(Boc-^{L}Trp)amino}propyl]hexadecanamide (29b)

FT-IR (NaCl): 3308 cm⁻¹ (—NH— str.), 2956 cm⁻¹ (aromatic C—H str.), 2920 cm⁻¹ (—CH₂— asym. str.), 2852 cm⁻¹ (—CH₂— sym. str.), 1720 cm⁻¹ (C═O str.), 1623 cm⁻¹, 1542 cm⁻¹ (aromatic C═C str.). ¹H-NMR (400 MHz, CDCl₃) δ/ppm: 8.512-8.289 (d, 2H), 7.484-6.664 (m, 12H), 5.297-5.224 (d, 2H), 4.423-4.405 (d, 2H), 3.351-2.832 (m, 12H), 2.111-2.092 (t, 2H), 1.551-1.461 (m, 6H). 1.364 (s, 18H), 1.248 (s, 24H), 0.894-0.860 (t, 3H). HRMS (m/z): 942.6387 [(M+H)⁺] (Observed), 942.6432 [(M+H)⁺] (Calculated).

### Example 1.3.2: Synthesis of N,N-bis-[{3-(^{L}Trp)amino}propyl]alkanamide bis(trifluoro acetate) (7, 8)

At first 1 equivalent of 29a and 29b were dissolved in DCM. Then to the intensely stirred solution 4 equivalents (excess amount) of trifluoroacetic acid (TFA) was added and allowed to stir at RT for 12 h. After that reaction solvent and unreacted TFA was removed to get the pure product 7, 8 with 100% yield.

### Example 1.3.2.1: N,N-bis-[{3-(^{L}Trp)amino}propyl]tetradecanamide bis(trifluoroacetate) (7)

FT-IR (NaCl): 3268 cm⁻¹ (—NH— str.), 3062 cm⁻¹ (aromatic C—H str.), 2925 cm⁻¹ (—CH₂— asym. str.), 2853 cm⁻¹ (—CH₂— sym. str.), 1673 cm⁻¹ (C═O str.), 1618 cm⁻¹, 1578 cm⁻¹ (aromatic C═C str.). ¹H-NMR (400 MHz, DMSO-d₆) δ/ppm: 11.013-10.666 (m, 2H), 8.548-8.100 (m, 8H), 7.635-6.979 (m, 10H), 3.902 (s, 2H), 3.219-3.033 (m, 12H), 2.193-2.161 (t, 2H), 1.425-1.319 (m, 6H), 1.223 (s, 20H), 0.865-0.831 (t, 3H). HRMS (m/z): 714.5039 [(M+H)⁺] (Observed), 714.5071 [(M+H)+](Calculated).

### Example 1.3.2.2: N,N-bis-[{3-(^{L}Trp)amino}propyl]hexadecanamide bis(trifluoroacetate) (8)

FT-IR (NaCl): 3262 cm-¹ (—NH— str.), 3058 cm-¹ (aromatic C—H str.), 2924 cm-¹ (—CH₂— asym. str.), 2853 cm-¹ (—CH₂— sym. str.), 1674 cm-¹ (C═O str.), 1618 cm-¹, 1578 cm-¹ (aromatic C═C str.). ¹H-NMR (400 MHz, DMSO-d₆) δ/ppm: 10.999-10.657 (m, 2H), 8.523-8.368 (m, 8H), 7.625-6.977 (m, 10H), 3.418 (s, 2H), 3.215-2.938 (m, 12H), 2.185-2.153 (t, 2H), 1.486-1.312 (m, 6H), 1.217 (s, 24H), 0.859-0.825 (t, 3H). HRMS (m/z): 742.5376 [(M+H)⁺] (Observed), 742.5384 [(M+H)+](Calculated).

### Example 1.4: Synthesis of N¹-(Boc-^{L}Lys-Boc)-N³-[{3-(Boc-^{L}Lys-Boc)amino}propyl]propane-1,3-diamine (30)

About 5.75 g (2 equivalents, 16.6 mmol) of N,N-Di-Boc-L-Lysine was dissolved in about 30 mL of dry DCM at 0° C. In the reaction mixture about 8.65 mL (6 equivalents, 49.8 mmol) of DIPEA was added followed by about 6.30 g (2 equivalents, 16.6 mmol) of HBTU. Now about 10 mL of DMF was added to the reaction mixture. After 10 minutes, about 1.2 g (1 equivalent, 8.3 mmol) of norspermidine [Bis (3-aminopropyl) amine] was added to the reaction mixture drop wise. The reaction mixture was allowed to stir for 48 h at 0° C. After that the reaction solvent was evaporated and residue was diluted in ethyl acetate. Thereafter work-up was done at first with 1N HCl (50 mL, 3 times) followed by saturated Na₂CO₃ solution (50 mL, 3 times). The crude product was collected in ethyl acetate layer. Finally column was done to isolate the product with 65% yield. FT-IR (NaCl): 3315 cm⁻¹ (—NH-str.), 2929 cm⁻¹ (—CH₂— asym. str.), 2866 cm⁻¹ (—CH₂— sym. str.), 1700 cm⁻¹, 1673 cm⁻¹ (C═O str.). ¹H-NMR (400 MHz, CDCl₃) δ/ppm: 7.475 (s, 2H), 5.521 (s, 2H), 4.786 (s, 2H), 4.034 (s, 2H), 3.514-3.008 (m, 12H), 2.049 (s, 1H), 1.773-1.486 (m, 16H), 1.425 (s, 36H). HRMS (m/z): 788.5493 [(M+H)⁺] (Observed), 788.5497 [(M+H)⁺] (Calculated).

### Example 1.4.1: Synthesis of N,N-bis-[{3-(Boc-^{L}Lys-Boc)amino}propyl]alkanamide (31a-31e)

About 1.5 equivalents of saturated aliphatic acid (C10, C12, C14, C16, and C18) were dissolved in dry DCM at 0° C. Then about 4 equivalents of DIPEA followed by about 1.5 equivalents of HBTU were added to it. Now DMF was added to the reaction mixture. After 10 minutes, about 1 equivalent of 30 was added drop wise dissolving it in dry DCM. The reaction mixture was brought to RT and allowed to stir for 24 h. Then reaction solvent was evaporated and residue was diluted in ethyl acetate. Thereafter work-up was done at first with 1N HCl (3 times) followed by saturated Na₂CO₃ solution (3 times). The crude product was collected in ethyl acetate layer. Finally column was done to isolate the puree product (31a-31e) with 75-80% yield.

### Example 1.4.1.1: N,N-bis-[{3-(Boc-^{L}Lys-Boc)amino}propyl]decanamide (31a)

FT-IR (NaCl): 3316 cm⁻¹ (—NH— str.), 2928 cm⁻¹ (—CH₂— asym. str.), 2855 cm⁻¹ (—CH₂— sym. str.), 1700 cm⁻¹, 1661 cm⁻¹ (C═O str.). H-NMR (400 MHz, CDCl₃) δ/ppm: 7.320 (s, 2H), 5.364-5.300 (d, 2H), 4.713 (s, 2H), 4.152-4.134 (d, 2H), 3.549-2.991 (m, 12H), 2.272-2.233 (t, 2H), 1.886-1.461 (m, 18H), 1.419 (s, 36H), 1.249 (s, 12H), 0.882-0.848 (t, 3H). HRMS (m/z): 942.6830 [(M+H)⁺] (Observed), 942.6855 [(M+H)⁺] (Calculated).

### Example 1.4.1.2: N,N-bis-[{3-(Boc-^{L}Lys-Boc)amino}propyl]dodecanamide (31b)

FT-IR (NaCl): 3320 cm⁻¹ (—NH— str.), 2929 cm⁻¹ (—CH₂— asym. str.), 2855 cm⁻¹ (—CH₂-sym. str.), 1695 cm⁻¹, 1661 cm⁻¹ (C═O str.). ¹H-NMR (400 MHz, CDCl₃) δ/ppm: 7.349 (s, 2H), 5.398-5.331 (d, 2H), 4.728 (s, 2H), 4.151-4.134 (d, 2H), 3.545-2.988 (m, 12H), 2.269-2.231 (t, 2H), 1.737-1.458 (m, 18H), 1.415 (s, 36H), 1.242 (s, 16H), 0.880-0.846 (t, 3H). HRMS (m/z): 970.7135 [(M+H)⁺] (Observed), 970.7168 [(M+H)⁺] (Calculated).

### Example 1.4.1.3 N,N-bis-[{3-(Boc-^{L}Lys-Boc)amino}propyl]tetradecanamide (31c)

FT-IR (NaCl): 3311 cm⁻¹ (—NH— str.), 2927 cm⁻¹ (—CH₂— asym. str.), 2855 cm⁻¹ (—CH₂— sym. str.), 1697 cm⁻¹, 1661 cm⁻¹ (C═O str.). ¹H-NMR (400 MHz, CDCl₃) δ/ppm: 7.327 (s, 2H), 5.65-5.302 (d, 2H), 4.710 (s, 2H), 4.156-4.139 (d, 2H), 3.560-2.966 (m, 12H), 2.275-2.236 (t, 2H), 1.667-1.464 (m, 18H), 1.423 (s, 36H), 1.247 (s, 20H), 0.888-0.854 (t, 3H). HRMS (m/z): 998.7462 [(M+H)⁺] (Observed), 998.7481 [(M+H)⁺] (Calculated).

### Example 1.4.1.4: N,N-bis-[{3-(Boc-^{L}Lys-Boc)amino}propyl]hexadecanamide (31d)

FT-IR (NaCl): 3337 cm⁻¹ (—NH— str.), 2929 cm⁻¹ (—CH₂— asym. str.), 2869 cm⁻¹ (—CH₂— sym. str.), 1697 cm⁻¹, 1661 cm⁻¹ (C═O str.). ¹H-NMR (400 MHz, CDCl₃) δ/ppm: 7.360-7.309 (d, 2H), 5.422-5.351 (d, 2H), 4.750 (s, 2H), 4.139-4.123 (d, 2H), 3.526-2.985 (m, 12H), 2.258-2.220 (t, 2H), 1.714-1.452 (m, 18H), 1.403 (s, 36H), 1.228 (s, 24H), 0.870-0.836 (t, 3H). HRMS (m/z): 1026.7787 [(M+H)⁺] (Observed), 1026.7794 [(M+H)⁺] (Calculated).

### Example 1.4.1.5: N,N-bis-[{3-(Boc-^{L}Lys-Boc)amino}propyl]octadecanamide (31e)

FT-IR (NaCl): 3321 cm⁻¹ (—NH— str.), 2927 cm⁻¹ (—CH₂— asym. str.), 2853 cm⁻¹ (—CH₂-sym. str.), 1699 cm⁻¹, 1663 cm⁻¹ (C═O str.). ¹H-NMR (400 MHz, CDCl₃) δ/ppm: 7.367-7.310 (d, 2H), 5.436-5.372 (d, 2H), 4.768 (s, 2H), 4.116-4.099 (d, 2H), 3.513-3.055 (m, 12H), 2.252-2.216 (t, 2H) 1.722-1.443 (m, 18H), 1.398 (s, 36H), 1.223 (s, 28H), 0.865-0.830 (t, 3H). HRMS (m/z): 1054.8097 [(M+H)⁺] (Observed), 1054.8107 [(M+H)+](Calculated).

### Example 1.4.2: Synthesis of N,N-bis-[{3-(^{L}Lys)amino}propyl]alkanamide bis(trifluoroacetate) (9-13)

At first 1 equivalent of 31a-31e were dissolved in DCM. Then to the intensely stirred solution 4 equivalents (excess amount) of trifluoroacetic acid (TFA) was added and allowed to stir at RT for 12 h. After that reaction solvent and unused TFA was removed to get the pure product 9-13 with 100% yield.

### Example 1.4.2.1: N,N-bis-[{3-(^{L}Lys)amino}propyl]decanamide bis(trifluoroacetate) (9)

FT-IR (NaCl): 3277 cm⁻¹ (—NH— str.), 2928 cm⁻¹ (—CH₂— asym. str.), 2858 cm⁻¹ (—CH₂-sym. str.), 1677 cm⁻¹ (C═O str.). ¹H-NMR (400 MHz, CDCl₃) δ/ppm: 8.619-7.877 (m, 14H), 3.709-3.653 (d, 2H), 3.646-2.760 (m, 12H), 2.241-2.222 (t, 2H), 1.709-1.312 (m, 18H), 1.241 (s, 12H), 0.871-0.837 (t, 3H). HRMS (m/z): 542.4752 [(M+H)⁺] (observed), 542.4758 [(M+H)⁺] (calculated).

### Example 1.4.2.2: N,N-bis-[{3-(^{L}Lys)amino}propyl]dodecanamide bis(trifluoroacetate) (10)

FT-IR (NaCl): 3264 cm⁻¹ (—NH— str.), 2929 cm⁻¹ (—CH₂— asym. str.), 2869 cm⁻¹ (—CH₂— sym. str.), 1676 (C═O str.). ¹H-NMR (400 MHz, CDCl₃) δ/ppm: 8.608-7.862 (m, 14H), 3.709 (s, 2H), 3.274-2.761 (m, 12H), 2.254-2.221 (t, 2H), 1.688-1.289 (m, 18H), 1.239 (s, 16H), 0.871-0.837 (t, 3H). HRMS (m/z): 570.5016 [(M+H)^{+]} (observed), 570.5071 [(M+H)⁺] (calculated).

### Example 1.4.2.3: N,N-bis-[{3-(^{L}Lys)amino}propyl]tetradecanamide bis(trifluoroacetate) (11)

FT-IR (NaCl): 3316 cm⁻¹ (—NH— str.), 2978 cm⁻¹ (—CH₂— asym. str.), 2867 cm⁻¹ (—CH₂— sym. str.), 1697 cm⁻¹ (C═O str.). ¹H-NMR (400 MHz, CDCl₃) δ/ppm: 8.599-7.841 (m, 14H), 3.706 (s, 2H), 3.285-2.759 (m, 12H), 2.240-2.220 (t, 2H), 1.686-1.324 (m, 18H), 1.236 (s, 20H), 0.869-0.835 (t, 3H). HRMS (m/z): 598.3578 [(M+H)⁺] (observed), 598.5384 [(M+H)⁺] (calculated).

### Example 1.4.2.4: N,N-bis-[{3-(^{L}Lys)amino}propyl]hexadecanamide bis(trifluoroacetate) (12)

FT-IR (NaCl): 3337 cm⁻¹ (—NH— str.), 2979 cm⁻¹, 2929 cm⁻¹ (—CH₂— asym. str.), 2869 cm⁻¹ (—CH₂— sym. str.), 1697 cm⁻¹, 1661 cm⁻¹ (C═O str.). ¹H-NMR (400 MHz, CDCl₃) δ/ppm: 8.600-7.842 (d, 14H), 3.706 (s, 2H), 3.288-2.759 (m, 12H), 2.258-2.221 (t, 2H), 1.687-1.325 (m, 18H), 1.237 (s, 24H), 0.870-0.836 (t, 3H). HRMS (m/z): 626.5669 [(M+H)⁺] (observed), 626.5697 [(M+H)⁺] (calculated).

### Example 1.4.2.5: N,N-bis-[{3-(^{L}Lys)amino}propyl]octadecanamide bis(trifluoroacetate) (13)

FT-IR (NaCl): 3272 cm⁻¹ (—NH— str.), 2979 cm⁻¹ (—CH₂— asym. str.), 2854 cm⁻¹ (—CH₂— sym. str.), 1676 cm⁻¹ (C═O str.). ¹H-NMR (400 MHz, CDCl₃) δ/ppm: 8.599-7.846 (m, 14H), 3.708 (s, 2H), 3.274-3.033 (m, 12H), 2.254-2.220 (t, 2H), 1.687-1.324 (m, 18H), 1.235 (s, 28H), 0.869-0.835 (t, 3H). HRMS (m/z): 654.5973 [(M+H)⁺] (observed), 654.6010 [(M+H)⁺] (calculated).

### Example 1.5: Synthesis of ^{N}-(Boc-^{L}Orn-Boc)-N³-[{3-(Boc-^{L}Orn-Boc)amino}propyl]pro pane-1,3-diamine (32)

About 5 g (2 equivalents, 15.04 mmol) of N-Boc-L-Ornithine was dissolved in about 25 mL of dry DCM at 0° C. In the reaction mixture about 7.8 mL (6 equivalents, 45.12 mmol) of DIPEA was added followed by about 5.70 g (2 equivalents, 15.04 mmol) of HBTU. Now about 6 mL of DMF was added to the reaction mixture. After 10 minutes, about 0.99 g (1 equivalent, 7.52 mmol) of norspermidine [Bis (3-aminopropyl) amine] was added to the reaction mixture drop wise. The reaction mixture was allowed to stir for 48 h at 0° C. Then reaction solvent was evaporated and residue was diluted in ethyl acetate. Thereafter work-up was done at first with 1N HCl (50 mL, 3 times) followed by saturated Na₂CO₃ (50 mL, 3 times) solution. The crude product was collected in ethyl acetate layer. Finally column was done to isolate the product of 66% yield. FT-IR (NaCl): 3311 cm⁻¹ (—NH— str.), 2927 cm⁻¹ (—CH₂-asym. str.), 2856 cm⁻¹ (—CH₂— sym. str.), 1700 cm⁻¹, 1678 cm⁻¹ (C═O str.). ¹H-NMR (400 MHz, CDCl₃) δ/ppm: 7.472 (s, 2H), 5.524 (s, 2H), 4.776 (s, 2H), 4.134 (s, 2H), 3.504-3.018 (m, 12H), 2.139 (s, 1H), 1.765-1.445 (m, 12H), 1.395 (s, 36H). HRMS (m/z): 760.5172 [(M+H)⁺] (Observed), 760.5184 [(M+H)⁺] (Calculated).

### Example 1.5.1: N,N-bis-[{3-(Boc-^{L}Orn-Boc)amino}propyl]alkanamide (33a, 33b)

About 1.5 equivalents of saturated aliphatic acid (C14, C16) were dissolved in dry DCM at 0° C. Then about 4 equivalents of DIPEA followed by about 1.5 equivalents of HBTU were added to it. Now DMF was added to the reaction mixture. After 10 minutes, about 1 equivalent of 32 was added drop wise dissolving it in dry DCM. The reaction mixture was brought to RT and allowed to stir for 24 h. Then reaction solvent was evaporated and residue was diluted in ethyl acetate. Thereafter work-up was done at first with 1N HCl (3 times) followed by saturated Na₂CO₃ solution (3 times). The crude product was collected in ethyl acetate layer. Finally column was done to isolate the pure product with 75-80% yield.

### Example 1.5.1.1: N,N-bis-[{3-(Boc-^{L}Orn-Boc)amino}propyl]tetradecanamide (33a)

FT-IR (NaCl): 3318 cm¹ (—NH— str.), 2928 cm⁻¹ (—CH₂— asym. str.), 2854 cm⁻¹ (—CH₂— sym. str.), 1698 cm⁻¹ (C═O str.). ¹H-NMR (400 MHz, CDCl₃) δ/ppm: 7.350 (s, 2H), 5.523-5.548 (d, 2H), 4.879 (s, 2H), 4.199-4.181 (d, 2H), 3.479-3.025 (m, 12H), 2.256-2.218 (t, 2H), 1.731-1.491 (m, 14H), 1.397 (s, 36H), 1.224 (s, 20), 0.864-0.8350 (t, 3H). HRMS (m/z): 970.7175 [(M+H)⁺] (observed), 970.7168 [(M+H)⁺] (calculated).

### Example 1.5.1.2: N,N-bis-[{3-(Boc-^{L}Orn-Boc)amino}propyl]hexadecanamide (33b)

FT-IR (NaCl): 3318 cm⁻¹ (—NH— str.), 2927 cm⁻¹ (—CH₂— asym. str.), 2855 cm⁻¹ (—CH₂— sym. str.), 1700 cm⁻¹ (C═O str.). ¹H-NMR (400 MHz, CDCl₃) δ/ppm: 7.284 (s, 2H), 5.459-5.387 (d, 2H), 4.849-4.833 (d, 2H), 4.216-4.199 (d, 2H), 3.501-3.040 (m, 12H), 2.270-2.232 (t, 2H), 1.767-1.541 (m, 14H), 1.415 (s, 36H), 1.241 (s, 24H), 0.882-0.848 (t, 3H). HRMS (m/z): 998.7415 [(M+H)⁺] (observed), 998.7481 [(M+H)⁺] (calculated).

### Example 1.5.2: Synthesis of N,N-bis-[{3-(^{L}Orn)amino}propyl]alkanamide bis(trifluoroacetate) (14, 15)

At first 1 equivalent of 33a and 33b were dissolved in DCM. Then to the intensely stirred solution 4 equivalents (excess amount) of trifluoroacetic acid (TFA) was added and allowed to stir at RT for 12 h. After that reaction solvent and unreacted TFA was removed to get the pure product 14, 15 with the yield of 100%.

### Example 1.5.2.1: N,N-bis-[{3-(^{L}Orn)amino}propyl]tetradecanamide bis(trifluoroacetate) (14)

FT-IR (NaCl): 3266 cm⁻¹ (—NH— str.), 2928 cm¹ (—CH₂-asym. str.), 2855 cm¹ (—CH₂— sym. str.), 1675 cm⁻¹ (C═O str.). ¹H-NMR (400 MHz, D₂O) 6/ppm: 4.048-4.010 (d, 2H), 3.367-3.043 (m, 12H), 2.373-2.236 (t, 2H), 2.001-1.576 (m, 14H), 1.294 (s, 20H), 0.906-0.873 (t, 3H). HRMS (m/z): 570.5063 [(M+H)⁺] (observed), 570.5071 [(M+H)⁺] (calculated).

### Example 1.5.2.2: N,N-bis-[{3-(^{L}Orn)amino}propyl]hexadecanamide bis(trifluoroacetate) (15)

FT-IR (NaCl): 3267 cm⁻¹ (—NH— str.), 2924 cm¹ (—CH₂-asym. str.), 2854 cm¹ (—CH₂— sym. str.), 1673 cm-⁻¹ (C═O str.). ¹H-NMR (400 MHz, D₂O) 8/ppm: 4.044-4.011 (d, 2H), 3.369-3.039 (m, 12H), 2.372-2.235 (t, 2H), 2.001-1.579 (m, 14H), 1.298 (s, 24H), 0.911-0.878 (t, 3H). HRMS (m/z): 598.5378 [(M+H)⁺] (observed), 598.5384 [(M+H)⁺] (calculated).

### Example 1.6: Synthesis of N¹-(Boc-^{D}Lys-Boc)-N³-[{3-(Boc-^{D}Lys-Boc)amino}propyl]propane-1,3-diamine (34)

About 5 g (2 equivalents, 14.4 mmol) of N,N-Di-Boc-D-Lysine was dissolved in about 25 mL of dry DCM at 0° C. Then to the reaction mixture about 7.5 mL (6 equivalents, 43.2 mmol) of DIPEA was added followed by about 6.30 g (2 equivalents, 14.4 mmol) of HBTU. Now about 6 mL of DMF was added to the reaction mixture. After another 10 minutes, about 0.94 g (1 equivalent, 8.3 mmol) of norspermidine [Bis (3-aminopropyl) amine] was added to the reaction mixture drop wise. Now, the reaction mixture was allowed to stir for 48 h at 0° C. Next, the reaction solvent was evaporated and residue was diluted in ethyl acetate. Thereafter work-up was done at first with 1N HCl (50 mL, 3 times) followed by saturated Na₂CO₃ solution (50 mL, 3 times). The crude product was collected in ethyl acetate layer. Finally column was done to isolate the product with 67% yield. FT-IR(NaCl): 3316 cm⁻¹ (—NH-str.), 2931 cm⁻¹ (—CH₂— asym. str.), 2863 cm⁻¹ (—CH₂— sym. str.), 1701 cm⁻¹, 1670 cm⁻¹ (C═O str.). ¹H-NMR (400 MHz, CDCl₃) δ/ppm: 7.475 (s, 2H), 5.521 (s, 2H), 4.786 (s, 2H), 4.034 (s, 2H), 3.514-3.008 (m, 12H), 2.049 (s, 1H), 1.773-1.486 (m, 16H), 1.425 (s, 36H). HRMS (m/z): 788.5497 [(M+H)⁺] (Observed), 788.5593 [(M+H)⁺] (Calculated).

### Example 1.6.1: Synthesis of N,N-bis-[{3-(Boc-^{D}Lys-Boc)amino}propyl]alkanamide (35a, 35b)

About 1.5 equivalents of saturated aliphatic acid (C14, C16) were dissolved in dry DCM at 0° C. Then about 4 equivalents of DIPEA and followed by about 1.5 equivalents of HBTU were added to the reaction mixture. Now DMF was added to the reaction mixture. After 10 minutes, about 1 equivalent of 34 was dissolved in dry DCM and added to the reaction mixture drop wise. Now, the reaction mixture was brought to RT and allowed to stir for 24 h. Then reaction solvent was evaporated and residue was diluted in ethyl acetate. Thereafter work-up was done at first with 1N HCl (3 times) followed by saturated Na₂CO₃ solution (3 times). The crude product was collected in ethyl acetate layer. Finally column was done to isolate the puree product with 75-80% yield.

### Example 1.6.1.1: N,N-bis-[{3-(Boc-^{D}Lys-Boc)amino}propyl]tetradecanamide (35a)

FT-IR(NaCl): 3311 cm⁻¹ (—NH— str.), 2927 cm⁻¹ (—CH₂— asym. str.), 2855 cm⁻¹ (—CH₂— sym. str.), 1697 cm⁻¹, 1661 cm⁻¹ (C═O str.). ¹H-NMR (400 MHz, CDCl₃) δ/ppm: 7.326 (s, 2H), 5.345-5.288 (d, 2H), 4.708 (s, 2H), 4.158-4.141 (d, 2H), 3.569-2.984 (m, 12H), 2.276-2.237 (t, 2H), 1.747-1.465 (m, 18H), 1.424 (s, 36H), 1.247 (s, 20H), 0.889-0.855 (t, 3H). HRMS (m/z): 998.7415 [(M+H)⁺] (observed), 998.7481 [(M+H)⁺] (calculated).

### Example 1.6.1.2: N,N-bis-[{3-(Boc-^{D}Lys-Boc)amino}propyl]hexadecanamide (35b)

FT-IR(NaCl): 3311 cm⁻¹ (—NH— str.), 2927 cm⁻¹ (—CH₂— asym. str.), 2855 cm⁻¹ (—CH₂— sym. str.), 1697 cm⁻¹, 1661 cm⁻¹ (C═O str.). ¹H-NMR (400 MHz, CDCl₃) δ/ppm: 7.361-7.309 (s, 2H), 5.435-5.363 (d, 2H), 4.53 (s, 2H), 4.120 (s, 2H), 3.529-2.978 (m, 12H), 2.256-2.218 (t, 2H), 1.712-1.446 (m, 18H), 1.402 (s, 36H), 1.226 (s, 20H), 0.868-0.834 (t, 3H). HRMS (m/z): 1026.7742 [(M+H)⁺] (observed), 1026.7794 [(M+H)⁺] (calculated).

### Example 1.6.2: Synthesis of N,N-bis-[{3-(^{D}Lys)amino}propyl]alkanamide bis(trifluoroacetate) (16, 17)

At first 1 equivalent of 35a and 35b were dissolved in DCM. Then to the intensely stirred solution, 4 equivalents (excess amount) of trifluoroacetic acid (TFA) was added and allowed to stir at RT for 12 h. After that reaction solvent and unreacted TFA was removed to get the pure product 16, 17 with the yield of 100%.

### Example 1.6.2.1: N,N-bis-[{3-(^{D}Lys)amino}propyl]tetradecanamide bis(trifluoroacetate) (16)

FT-IR (NaCl): 3268 cm⁻¹ (—NH— str.), 2927 cm⁻¹ (—CH₂-asym. str.), 2857 cm⁻¹ (—CH₂— sym. str.), 1676 cm⁻¹ (C═O str.). ¹H-NMR (400 MHz, D₂O) 8/ppm: 3.983-3.976 (m, 2H), 3.352-2.984 (m, 12H), 2.350-2.314 (t, 2H), 1.923-1.445 (m, 18H), 1.280 (s, 24H), 0.894-0.862 (t, 3H). HRMS (m/z): 598.5378 [(M+H)⁺] (observed), 598.5384 [(M+H)⁺] (calculated).

### Example 1.6.2.2: N,N-bis-[{3-(^{D}Lys)amino}propyl]hexadecanamide bis(trifluoroacetate) (17)

FT-IR (NaCl): 3272 cm⁻¹ (—NH— str.), 2926 cm⁻¹ (—CH₂-asym. str.), 2855 cm⁻¹ (—CH₂— sym. str.), 1677 cm⁻¹ (C═O str.). ¹H-NMR (400 MHz, D₂O) 8/ppm: 4.007-3.979 (m, 2H), 3.370-3.002 (m, 12H), 2.372-2.336 (t, 2H), 1.924-1.462 (m, 18H), 1.300 (s, 24H), 0.913-0.880 (t, 3H). HRMS (m/z): 626.5688 [(M+H)⁺] (observed), 626.5697 [(M+H)⁺] (calculated).

### Example 1.7: Synthesis of N,N-bis-(propylnitrile)octylamine (36)

At first, about 5 g (1 equivalent, 38.68 mmol) of N-octyl amine was dissolved in about 25 mL of dry MeOH at 0° C. Then to the reaction mixture about 15.2 mL (6 equivalents, 232.08 mmol) of acrylonitrile was added drop wise during 30 minutes. Now the reaction mixture was brought to RT and kept for stirring for 48 h. Then the reaction solvent and unused acrylonitrile was removed by using rotary evaporator, finally using high vacuum pump and oven to get the pure 36 with the yield of 100%. FT-IR (NaCl): 2926 cm⁻¹ (—CH₂-asym. str.), 2856 cm⁻¹ (—CH₂— sym. str.), 2249 cm⁻¹ (—CN str.). ¹H-NMR (400 MHz, CDCl₃) δ/ppm: 2.873-2.839 (t, 4H), 2.536-2.499 (t, 2H), 2.477-2.442 (t, 4H), 1.460-1.281 (m, 2H), 1.277 (s, 10H), 0.897-0.862 (t, 3H).

### Example 1.7.1: Synthesis of N¹-(3-aminopropyl)-N¹-octylpropane-1,3-diamine tris(trifluoroacetate) (37)

About 2.88 g (2 equivalents, 75.88 mmol) of lithium aluminium hydride (LAH) was made suspension in about 20 mL of freshly dried diethyl ether (Et₂O) at 0° C. under nitrogen atmosphere. Now, about 8.93 g (1 equivalent, 37.94 mmol) of 36 was dissolved in about 10 mL of dry Et₂O and inject into the LAH suspension drop wise. Then, the reaction mixture was brought at RT and allowed to stir for 2 h. Now, again the reaction mixture was brought to 0° C. and milipore water was added to it slowly drop wise. After complete destruction of unreacted LAH, workup has done with water and Et₂O. The organic layer (Et₂O) was collected several times and finally dried over anhydrous sodium sulphate (Na₂SO₄). Then it was evaporated to get the crude product. Now, crude was made suspension in 20 mL of 4N HCl and allowed to stir for 2 h. Then, again workup was done using water and Et₂O, aqueous layer was collected and volume was reduced to 5 mL using rotary evaporator. Finally, remaining water was removed by using lyophilizer to get the pure 37 with the yield of 60%. FT-IR (NaCl): 3357 cm⁻¹ (—NH— str.), 2927 cm⁻¹ (—CH₂— asym. str.), 2854 cm⁻¹ (—CH₂— sym. str.). ¹H-NMR (400 MHz, D₂O) δ/ppm: 3.349-3.307 (t, 4H), 3.266-3.225 (t, 2H), 3.138-3.099 (t, 4H), 2.195-2.115 (m, 4H), 1.756-1.700 (m, 2H), 1.299 (s, 10H), 0.898-0.865 (t, 3H). HRMS (m/z): 244.2774 [(M+H)⁺] (observed), 244.2753 [(M+H)⁺] (calculated).

### Example 1.7.2: N¹-octyl-N³-(Boc-^{L}AA)-N¹-[3-{(Boc-^{L}AA)amino}propyl]propane-1,3-diamine (38a-38c)

About 2 equivalents of respective N-Boc-amino acid (N-Boc-L-Phenylalanine, N-Boc-L-Alanine and N,N-Di-Boc-L-Lysine) was dissolved in dry DCM at 0° C. Then about 3 equivalents of DIPEA were added to the reaction mixture followed by about 2 equivalents of HBTU. Now DMF was added to the reaction mixture. After 10 minutes, about 1 equivalent of 37 was added drop wise dissolving it in dry DCM and another 3 equivalents of DIPEA. The reaction mixture was brought to RT and allowed to stir for 48 h. Then reaction solvent was evaporated and residue was diluted in ethyl acetate. Thereafter work-up was done at first with 1N HCl (3 times) followed by saturated Na₂CO₃ solution (3 times). The crude product was collected in ethyl acetate layer. Finally column was done to isolate the puree product with 70-75% yield.

### Example 1.7.2.1: N¹-octyl-N³-(Boc-^{L}Phe)-N¹-[3-{(Boc-^{L}Phe)amino}propyl]propane-1,3-diamine (38a): FT-IR (NaCl)

3264 cm¹ (—NH— str.), 3033 cm⁻¹ (aromatic C—H str.), 2930 cm⁻¹ (—CH₂— asym. str.), 2856 cm⁻¹ (—CH₂— sym. str.), 1700 cm⁻¹ (C═O str.), 1674 cm⁻¹ (aromatic C═C str.). ¹H-NMR (400 MHz, CDCl₃) δ/ppm: 7.219-7.190 (m, 12H), 5.451-5.258 (m, 2H), 4.363-4.348 (d, 2H), 3.375-2.884 (m, 14H), 1.852-1.328 (m, 6H), 1.278 (s, 18H), 1.265 (s, 10H), 0.881-0.847 (t, 3H).

### Example 1.7.2.2: N¹-octyl-N³-(Boc-^{L}Ala)-N¹-[3-{(Boc-^{L}Ala)amino}propyl]propane-1,3-diamine (38b)

FT-IR (NaCl): 3294 cm⁻¹ (—NH— str.), 2929 cm⁻¹ (—CH₂— asym. str.), 2856 cm⁻¹ (—CH₂— sym. str.), 1700 cm⁻¹ 1659 cm⁻¹ (C═O str.). ¹H-NMR (400 MHz, CDCl3) δ/ppm: 7.301 (s, 2H), 5.542 (s, 2H), 4.191 (s, 2H), 3.489-3.191 (m, 4H), 2.613-2.553 (m, 6H), 1.723-1.674 (d, 6H), 1.437 (s, 18H), 1.369 (s, 6H), 1.269 (s, 10H), 0.895-0.861 (t, 3H).

### Example 1.7.2.3: N¹-octyl-N³-(Boc-^{L}Lys)-N¹-[3-{(Boc-^{L}Lys)amino}propyl]propane-1,3-diamine (38c)

FT-IR (NaCl): 3321 cm⁻¹ (—NH— str.), 2928 cm⁻¹ (—CH₂— asym. str.), 2856 cm⁻¹ (—CH₂— sym. str.), 1700 cm⁻¹ 1664 cm⁻¹ (C═O str.). ¹H-NMR (400 MHz, CDCl₃) δ/ppm: 7.146 (s, 2H), 5.295-5224 (d, 2H), 4.695-4.507 (d, 2H), 4.157-4.076 (d, 2H), 3.600-3.098 (m, 14H), 2.019-1.490 (m, 18H), 1.434 (s, 36H), 1.272 (s, 10H), 0.898-0.871 (t, 3H).

### Example 1.7.3.1: N¹-octyl-N³-(Boc-^{L}Phe)-N¹-[3-{(Boc-^{L}Phe)amino}propyl]propane-1,3-diamine tris(trifluoroacetate) (18)

FT-IR (NaCl): 3222 cm⁻¹ (—NH— str.), 3030 cm¹ (aromatic C—H str.), 2930 cm⁻¹ (—CH₂— asym. str.), 2856 cm⁻¹ (—CH₂— sym. str.), 1674 cm⁻¹ (C═O str.), 1574 cm⁻¹ (aromatic C═C str.). ¹H-NMR (400 MHz, DMSO-d₆) δ/ppm: 8.557-8.197 (m, 7H), 7.344-7.222 (m, 10H), 3.946-3.932 (d, 2H), 3.167-2.861 (m, 14H), 1.617-1.525 (m, 6H), 1.265 (s, 10H), 0.870-0.839 (t, 3H). HRMS (m/z): 538.4098 [(M+H)⁺] (observed), 538.4121 [(M+H)⁺] (calculated).

### Example 1.7.3.2: N¹-octyl-N³-(Boc-^{L}Ala)-N¹-[3-{(Boc-^{L}Ala)amino}propyl]propane-1,3-diamine tris(trifluoroacetate) (19)

FT-IR (NaCl): 3210 cm⁻¹ (—NH— str.), 2928 cm⁻¹ (—CH₂— asym. str.), 2857 cm⁻¹ (—CH₂— sym. str.), 1675 cm⁻¹ (C═O str.). ¹H-NMR (400 MHz, DMSO-d₆) δ/ppm: 8.714-8.219 (d, 7H), 3.836-3.819 (d, 2H), 3.210-2.850 (m, 14H), 1.907-1.618 (m, 6H), 1.349 (s, 6H) 1.269 (s, 10H), 0.882-0.848 (t, 3H). HRMS (m/z): 386.3406 [(M+H)⁺] (observed), 386.3495 [(M+H)⁺] (calculated).

### Example 1.7.3.3: N¹-octyl-N³-(Boc-^{L}Lys)-N¹-[3-{(Boc-^{L}Lys)amino}propyl]propane-1,3-diamine tris(trifluoroacetate) (20)

FT-IR (NaCl): 3210 cm⁻¹ (—NH— str.), 2926 cm⁻¹ (—CH₂— asym. str.), 2856 cm⁻¹ (—CH₂— sym. str.), 1680 cm⁻¹ (C═O str.). ¹H-NMR (400 MHz, DMSO-d₆) δ/ppm: 8.691-7.877 (m, 13H), 3.731-3.716 (d, 2H), 3.700-2.737 (m, 14H), 1.801-1.341 (m, 18H), 1.280 (s, 10H), 0.886-0.852 (t, 3H). HRMS (m/z): 500.4658 [(M+H)⁺] (observed), 500.4652 [(M+H)⁺] (calculated).

### Example 1.8: Synthesis of N,N-bis-[{3-(^{L}Phe)amino}propyl]α-naphthylacetamide bis(trifluoroacetate) (21)

About 2.23 g (1.5 equivalents, 1.2 mmol) of α-Naphthyl acetic acid was dissolved in about 4 mL of dry DCM at 0° C. To the reaction mixture about 0.63 mL (4.5 equivalents, 3.56 mmol) of DIPEA was added followed by about 0.45 (1.5 equivalents, 1.2 mmol) of HBTU. Now about 1 mL of DMF was added to the reaction mixture. After 10 minutes, about 0.5 g (1 equivalent, 8.3 mmol) of 24 was added to the reaction mixture drop wise dissolving it in about 0.5 mL of dry DCM. The reaction mixture was allowed to stir for 48 h at 0° C. After that the reaction solvent was evaporated and residue was diluted in ethyl acetate. Thereafter work-up was done at first with 1N HCl (10 mL, 3 times) followed by saturated Na₂CO₃ solution (10 mL, 3 times). The crude product was collected in ethyl acetate layer. Now the ethyl acetate was removed by using rotary evaporator and crude product was dissolved again in 2 mL of DCM followed by 2 mL of trifluoroacetic acid (TFA) was added to the solution. After 12 h reaction solvent and unused TFA was removed by rotary evaporator, finally using high vacuum pump. Then it was dissolved in milipore water and HPLC has done to get the pure 21 with 75% yield. FT-IR(NaCl): 3315 cm⁻¹ (—NH— str.), 2929 cm⁻¹ (—CH₂— asym. str.), 2866 cm⁻¹ (—CH₂— sym. str.), 1700 cm⁻¹, 1673 cm⁻¹ (C═O str.). ¹H-NMR (400 MHz, D₂O) 8/ppm: 7.887-7.118 (m, 17H), 4.105-4,073 (m, 2H), 3.980-3.969 (d, 2H), 3.200-2.891 (m, 12H), 1.498-1.446 (m, 4H). HRMS (m/z): 594.3410 [(M+H)⁺] (Observed), 594.3444 [(M+H)⁺](Calculated).

### Example 1.9: Synthesis of N¹-^{L}Phe-N³-[{3-(^{L}Phe)amino}propyl]propane-1,3-diamine tris(trifluoroacetate) (22)

At first 1 equivalent of 24 was dissolved in DCM. To the intensely stirred solution 4 equivalents (excess amount) of trifluoroacetic acid (TFA) was added and allowed to stir at RT for 12 h. After that reaction solvent and unused TFA was removed to get the pure product with 100% yield. FT-IR (NaCl): 3273 cm⁻¹ (—NH— str.), 3036 cm⁻¹ (aromatic C—H str.), 2924 cm⁻¹ (—CH₂— asym. str.), 2857 cm⁻¹ (—CH₂— sym. str.), 1678 cm⁻¹ (C═O str.), 1617 cm⁻¹, 1579 cm⁻¹ (aromatic C═C str.). ¹H-NMR (400 MHz, DMSO-d₆) δ: 8.502-8.242 (m, 10H), 7.360-7.214 (m, 10H), 3.927-3.912 (d, 2H), 3.171-2.719 (m, 12H), 1.644-1.614 (m, 4H). HRMS (m/z): 426.2879 [(M+H)⁺] (Observed), 426.2869 [(M+H)⁺] (Calculated).

### Example 1.10: Synthesis of N¹-^{L}Lys-N³-[{3-(^{L}Lys)amino}propyl]propane-1,3-diamine tris(trifluoroacetate) (23)

At first 1 equivalent of 30 was dissolved in DCM. To the intensely stirred solution 4 equivalents (excess amount) of trifluoroacetic acid (TFA) was added and allowed to stir at RT for 12 h. After that reaction solvent and unused TFA was removed to get the pure product with 100% yield. FT-IR (NaCl): 3321 cm⁻¹ (—NH— str.), 2927 cm⁻¹ (—CH₂— asym. str.), 2846 cm⁻¹ (—CH₂— sym. str.), 1710 cm¹, 1678 cm⁻¹ (C═O str.). ¹H-NMR (400 MHz, DMSO-d₆) δ/ppm: 8.613-7.866 (m, 16H), 4.011 (s, 2H), 3.512-3.014 (m, 12H), 1.713-1.416 (m, 16H). HRMS (m/z): 388.3409 [(M+H)⁺] (Observed), 388.3400 [(M+H)⁺] (Calculated).

### Example 1.11: Synthesis of N,N-bis-[{3-(Boc-^{L}Lys-Boc)amino}propyl]alkenamide (41a, 41b)

About 1.5 equivalents of oleic and linoleic were dissolved in dry DCM at 0° C. Then about 4 equivalents of DIPEA followed by about 1.5 equivalents of HBTU were added to it. Now DMF was added to the reaction mixture. After 10 minutes, about 1 equivalent of 30 was added drop wise dissolving it in dry DCM. The reaction mixture was brought to RT and allowed to stir for 24 h. Then reaction solvent was evaporated and residue was diluted in ethyl acetate. Thereafter work-up was done at first with 1N HCl (3 times) followed by saturated Na₂CO₃ solution (3 times). The crude product was collected in ethyl acetate layer. Finally column was done to isolate the puree product (41a-41b) with 75-80% yield.

### Example 1.11.1.1: N,N-bis-[{3-(Boc-^{L}Lys-Boc)amido}propyl]oleamide (41a)

Yield-77%, FT-IR (NaCl): 3321 cm⁻¹ (—NH— str.), 2927 cm⁻¹ (—CH₂— asym. str.), 2853 cm⁻¹ (—CH₂— sym. str.), 1699 cm⁻¹, 1663 cm⁻¹ (C═O str.). ¹H-NMR (400 MHz, CDCl₃) δ/ppm: 7.359 (s, 2H), 5.481-5.292 (m, 4H), 4.764 (s, 2H), 4.130-4.117 (t, 2H), 3.436-3.049 (m, 12H), 2.250-2.212 (t, 2H), 1.980-1.949 (t, 4H), 1.717-1.438 (m, 18H), 1.394 (s, 36H), 1.2267-1.232 (bs, 20H), 0.861-0.827 (t, 3H); HRMS (m/z): 1052.7944 [(M+H)^{+]} (observed), 1052.7950 [(M+H)⁺] (calculated).

### Example 1.11.1.2: N,N-bis-[{3-(Boc-^{L}Lys-Boc)amido}propyl]linoleamide (41b)

Yield-76%. FT-IR (NaCl): 3321 cm⁻¹ (—NH— str.), 2927 cm⁻¹ (—CH₂— asym. str.), 2853 cm⁻¹ (—CH₂— sym. str.), 1699 cm⁻¹, 1663 cm⁻¹ (C═O str.); ¹H-NMR (400 MHz, CDCl₃) δ/ppm: 7.348 (s, 2H), 5.459-5.257 (m, 6H), 4.760 (s, 2H), 4.115 (t, 2H), 3.514-2.973 (m, 12H), 2.750-2.718 (t, 2H), 2.251-2.212 (t, 2H), 2.038-1.987 (m, 4H), 1.704-1.442 (m, 18H), 1.394 (s, 36H), 1.237 (bs, 14H), 0.871-0.836 (t, 3H); HRMS (m/z): 1050.7785 [(M+H)⁺] (observed), 1050.7794 [(M+H)⁺] (calculated).

### Example 1.11.2: Synthesis of N,N-bis-[{3-(^{L}Lys)amido}propyl]alkenamide tetrakis(trifluoroacetate) (39, 40)

About 1 equivalent of 41a and 41b was dissolved in DCM (3 mL). To the intensely stirred solution 4 equivalents (excess amount) of trifluoroacetic acid (TFA) was added and allowed to stirring at RT for 12 h. Then solvent and unused TFA were removed to afford pure products with 100% yield.

### Example 1.11.2.1: N,N-bis-[{3-(^{L}Lys)amido}propyl]oleamide tetrakis(trifluoroacetate) (39)

FT-IR (NaCl): 3272 cm⁻¹ (—NH— str.), 2979 cm⁻¹ (—CH₂-asym. str.), 2854 cm⁻¹ (—CH₂— sym. str.), 1676 cm⁻¹ (C═O str.). ¹H-NMR (400 MHz, DMSO-d₆) δ/ppm: 8.621-7.884 (m, 14H), 3.704 (t, 2H), 3.606 (m, 2H), 3.245-2.755 (m, 12H), 2.245-2.212 (t, 2H), 1.684-1.224 (m, 42H), 0.854-0.821 (t, 3H). HRMS (m/z): 652.5852 [(M+H)⁺] (observed), 652.5853 [(M+H)⁺] (calculated).

### Example 1.11.2.2: N,N-bis-[{3-(^{L}Lys)amido}propyl]linoleamide tetrakis(trifluoroacetate) (40)

FT-IR (NaCl): 3272 cm⁻¹ (—NH— str.), 2979 cm⁻¹ (—CH₂-asym. str.), 2854 cm⁻¹ (—CH₂— sym. str.), 1676 cm⁻¹ (C═O str.); ¹H-NMR (400 MHz, DMSO-d₆) δ/ppm: 8.605-7.850 (m, 14H), 5.500-5.050 (m, 2H), 3.708 (t, 2H), 3.253-2.758 (m, 14H), 2.237-2.220 (t, 2H), 2.026-1.954 (m, 2H), 1.687-1.240 (m, 36H), 0.850 (t, 3H). HRMS (m/z): 650.5684 [(M+H)⁺] (observed), 650.5697 [(M+H)⁺] (calculated).

### Example 1.12: N¹—Boc-N³-(3-(Boc-amino)propyl)propane-1,3-diamine (66)

About 1 equivalent of norspermidine was dissolved in MeOH (50 mL) and the solution was kept at −80° C. Then 1.5 equivalents of di-tert-butyldicarbonate (Boc₂O) was dissolved in MeOH (50 mL) and added to the reaction mixture drop wise. The reaction was continued for 1 h at −80° C. Then the reaction mixture was allowed to come at RT. MeOH was removed under reduced pressure and purification was done through column chromatography on silica gel (60-120 mesh) using methanol and chloroform (7:93) as eluent to afford the product with 65% yield. ¹H-NMR (400 MHz, CDCl₃) δ/ppm: 5.182 (s, 2H), 3.190-3.175 (m, 4H), 2.649-2.616 (m, 4H), 1.860 (s, 1H), 1.666-1.602 (m, 4H), 1.417 (s, 18H).

### Example 1.12.1: N,N-bis(3-(Boc-amino)propyl)alkanamide (67a-67c)

About 1.5 equivalents of saturated aliphatic acid (dodecanoic, tetradecanoic or hexadecanoic) were dissolved in dry DCM (12 mL) at 0° C. In the reaction mixture 4 equivalents of N,N-diisopropylethylamine (DIPEA) was added and followed by 1.5 equivalents of N,N,N′,N′-tetramethyl-O-(1H-benzotriazol-1-yl)uronium hexafluorophosphate (HBTU). Then DMF (3 mL) was added to the reaction mixture. After 10 minutes, 1 equivalent of 66 in dry DCM (1 mL) was added drop wise. The reaction mixture was brought to R. T. and allowed to stir for 24 h. Solvent was evaporated and residue was diluted in ethyl acetate (50 mL). Then work-up was carried out at first with 1N HCl (3 times) followed by saturated Na₂CO₃ solution (3 times). The crude product was extracted in ethyl acetate layer. Finally purification was accomplished through column chromatography on silica gel (60-120 mesh) using different ratios of methanol and chloroform as eluent to afford 67a-67c with 75-80% yield.

### Example 1.12.1.1: N,N-bis(3-(Boc-amino)propyl)dodecanamide (67a)

Yield-80%, ¹H-NMR (400 MHz, CDCl₃) δ/ppm: 5.379-4.629 (d, 2H), 3.400-3.029 (m, 8H), 2.296-2.258 (t, 2H), 1.764-1.594 (m, 6H), 1.422 (s, 18H), 1.249 (m, 16H), 0.887-0.853 (t, 3H).

### Example 1.12.1.2: N,N-bis(3-(Boc-amino)propyl)tetradecanamide (67b)

Yield-75%, ¹H-NMR (400 MHz, CDCl₃) δ/ppm: 5.377-4.643 (d, 2H), 3.400-3.030 (m, 8H), 2.299-2.261 (t, 2H), 1.774-1.592 (m, 6H), 1.422 (s, 18H), 1.246 (m, 20H), 0.887-0.853 (t, 3H).

### Example 1.12.1.3: N,N-bis(3-(Boc-amino)propyl)hexadecanamide (67c)

Yield-76%, ¹H-NMR (400 MHz, CDCl₃) δ/ppm: 5.377-4.643 (d, 2H), 3.400-3.030 (m, 8H), 2.299-2.261 (t, 2H), 1.774-1.592 (m, 6H), 1.422 (s, 18H), 1.246 (m, 24H), 0.887-0.853 (t, 3H).

### Example 1.17.2: N,N-bis(3-aminopropyl) alkanamide bis(trifluoroacetate) (68a-68c)

About 1 equivalent of 67a-67c was dissolved in DCM (3 mL). To the intensely stirred solution 4 equivalents (excess amount) of trifluoroacetic acid (TFA) was added and the mixture was stirred at R. T. for 12 h. Then solvent and unused TFA were removed to afford pure compounds 68a-68c with 100% yield.

### Example 1.12.2.1: N,N-bis(3-aminopropyl)dodecanamide bis(trifluoroacetate) (68a)

¹H-NMR (400 MHz, DMSO-d₆) δ/ppm: 7.931-7.797 (d, 6H), 3.326-3.268 (m, 4H), 2.832-2.688 (m, 4H), 2.299-2.262 (t, 2H), 1.821-1.693 (m, 6H), 1.502-1.470 (m, 2H), 1.242 (m, 16H), 0.871-0.837 (t, 3H). HRMS (m/z): 314.3172 [(M+H)⁺] (observed), 314.3171 [(M+H)⁺] (calculated).

### Example 1.12.2.2: N,N-bis(3-aminopropyl)tetradecanamide bis(trifluoroacetate) (68b)

¹H-NMR (400 MHz, DMSO-d₆) δ/ppm: 7.966-7.825 (d, 6H), 3.328-3.269 (m, 4H), 2.851-2.710 (m, 4H), 2.297-2.260 (t, 2H), 1.787-1.713 (m, 4H), 1.483 (m, 2H), 1.234 (m, 20H), 0.867-0.833 (t, 3H). HRMS (m/z): 342.3482 [(M+H)⁺] (observed), 342.3484 [(M+H)⁺] (calculated).

### Example 1.12.2.3: N,N-bis(3-aminopropyl)hexadecanamide bis(trifluoroacetate) (68c)

¹H-NMR (400 MHz, DMSO-d₆) δ/ppm: 7.890-7.765 (d, 6H), 3.328-3.272 (m, 4H), 2.851-2.713 (m, 4H), 2.304-2.267 (t, 2H), 1.801-1.695 (m, 4H), 1.491 (m, 2H), 1.241 (m, 24H), 0.875-0.840 (t, 3H); HRMS (m/z): 370.3787 [(M+H)⁺] (observed), 370.3797 [(M+H)^{+]} (calculated).

### Example 1.13.1: N,N-bis-[{3-(Boc-Gly)amido}propyl]alkanamide (69a-69c)

About 2.4 equivalents of N-Boc-Glycine was dissolved in dry DCM (12 mL) at 0° C. In the reaction mixture 6 equivalents of DIPEA was added followed by 2.4 equivalents of HBTU. Then DMF (3 mL) was added to the reaction mixture. After 10 minutes, 1 equivalent of 68a-68c in dry DCM (1 mL) was added drop wise. The reaction mixture was brought to RT and allowed to stir for 48 h. Solvent was evaporated and residue was diluted in ethyl acetate (50 mL). Then work-up was carried out at first with 1N HCl (3 times) followed by saturated Na₂CO₃ solution (3 times). The crude product was extracted in ethyl acetate layer. Finally purification was accomplished through column chromatography on silica gel (60-120 mesh) using methanol and chloroform as eluent to afford 69a-69c with 65-70% yield.

### Example 1.13.1.1: N,N-bis-[{3-(Boc-Gly)amido}propyl]dodecanamide (69a)

Yield: 67%, ¹H-NMR (400 MHz, CDCl₃) δ/ppm: 7.277-6.995 (s, 2H), 5.609 (s, 2H), 3.746-3.732 (d, 4H), 3.316-3.152 (m, 8H), 2.259-2.221 (t, 2H), 1.757-1.542 (m, 6H), 1.403 (s, 18H), 1.217 (bs, 16H), 0.853-0.819 (t, 3H). HRMS (m/z): 628.4653 [(M+H)^{+]} (observed), 628.4649 [(M+H)⁺] (calculated).

### Example 1.13.1.2: N,N-bis-[{3-(Boc-Gly)amido}propyl]tetradecanamide (69b)

Yield: 68%, ¹H-NMR (400 MHz, CDCl₃) δ/ppm: 7.225-6.874 (s, 2H), 5.520 (s, 2H), 3.766-3.751 (d, 4H), 3.338-3.160 (m, 8H), 2.277-2.239 (t, 2H), 1.774-1.564 (m, 6H), 1.425 (s, 18H), 1.234 (bs, 3H). HRMS (m/z): 656.4957 [(M+H)⁺] (observed), 656.4962 [(M+H)⁺] (calculated)

### Example 1.13.1.3: N,N-bis-[{3-(Boc-Gly)amido}propyl]hexadecanamide (69c): Yield

66%, ¹H-NMR (400 MHz, CDCl₃) δ/ppm: 7.229-6.874 (s, 2H), 5.511 (s, 2H), 3.769-3.754 (t, 2H), 3.340-3.163 (m, 8H), 2.278-2.240 (t, 2H), 1.775-1.583 (m, 6H), 1.427 (s, 18H), 1.234 (bs, 24H), 0.875-0.841 (t, 3H). HRMS (m/z): 684.5290 [(M+H)+](observed), 684.5275 [(M+H)⁺] (Calculated).

### Example 1.13.2: N,N-bis-[{3-(Gly)amido}propyl]alkanamide bis(trifluoroacetate) (42, 43, 44)

About 1 equivalent of 69a-69c was dissolved in DCM (3 mL). To the intensely stirred solution 4 equivalents (excess amount) of trifluoroacetic acid (TFA) was added and allowed to stirring at RT for 12 h. Then solvent and unused TFA were removed to afford products with 100% yield.

### Example 1.13.2.1: N,N-bis-[{3-(Gly)amido}propyl]dodecanamide bis(trifluoroacetate) (42)

¹H-NMR (100 MHz, DMSO-d₆); δ/ppm: 8.482-8.073 (s, 8H), 3.536-3.366 (s, 4H), 3.251-3.086 (m, 8H), 2.254-2.217 (t, 2H), 1.675-1.577 (m, 6H), 1.237 (bs, 16H), 0.868-0.834 (t, 3H). HRMS (m/z): 428.3588 [(M+H)⁺] (observed), 428.3601 [(M+H)⁺] (calculated).

### Example 1.13.2.2: N,N-bis-[{3-(Gly)amido}propyl]tetradecanamide bis(trifluoroacetate) (43)

¹H-NMR (400 MHz, DMSO-d₆) δ/ppm: 8.449-8.053 (s, 8H), 3.532-3.527 (s, 4H), 3.355-3.070 (m, 8H), 2.254-2.218 (t, 2H), 1.657-1.578 (m, 6H), 1.236 (bs, 20H), 0.852-0.835 (t, 3H). HRMS (m/z): 456.3915 [(M+H)⁺] (observed), 456.3914 [(M+H)⁺] (calculated).

### Example 1.13.2.3: N,N-bis-[{3-(Gly)amido}propyl]hexadecanamide bis(trifluoroacetate) (44)

¹H-NMR (400 MHz, DMSO-d₆) δ/ppm: 8.460-8.071 (s, 8H), 3.534-3.527 (s, 4H), 3.333-3.070 (m, 8H), 2.252-2.218 (t, 2H), 1.657-1.595 (m, 6H), 1.235 (bs, 24H), 0.851-0.835 (t, 3H). HRMS (m/z): 484.4231 [(M+H)⁺] (observed), 484.4227 [(M+H)⁺] (calculated).

### Example 1.14.1: N,N-bis-[{3-(Boc-^{L}Ala)amido}propyl]alkanamide (70a-70c)

About 2.4 equivalents of N-Boc-L-Alanine was dissolved in dry DCM (12 mL) at 0° C. In the reaction mixture 6 equivalents of DIPEA was added followed by 2.4 equivalents of HBTU. Then DMF (3 mL) was added to the reaction mixture. After 10 minutes, 1 equivalent of 68a-68c in dry DCM (1 mL) was added drop wise. The reaction mixture was brought to RT and allowed to stir for 48 hours. Solvent was evaporated and residue was diluted in ethyl acetate (50 mL). Then work-up was carried out at first with 1N HCl (3 times) followed by saturated Na₂CO₃ solution (3 times). The crude product was extracted in ethyl acetate layer. Finally purification was accomplished through column chromatography on silica gel (60-120 mesh) using methanol and chloroform as eluent to afford 70a-70c with 65-70% yield.

### Example 1.14.1.1: N,N-bis-[{3-(Boc-^{L}Ala)amido}propyl]dodecanamide (70a): Yield

65%, ¹H-NMR (400 MHz, CDCl₃) δ/ppm: 7.334-7.200 (d, 2H), 5.385-5.266 (m, 2H), 4.212-4.197 (d, 2H), 3.450-3.033 (m, 8H), 2.267-2.229 (t, 2H), 1.757-1.567 (m, 6H), 1.412 (s, 18H), 1.332-1.315 (d, 6H), 1.234 (bs, 16H), 0.872-0.838 (t, 3H). HRMS (m/z): 656.4949 [(M+H)⁺] (observed), 656.4962 [(M+H)⁺] (calculated).

### Example 1.14.1.2: N,N-bis-[{3-(Boc-^{L}Ala)amido}propyl]tetradecanamide (70b)

Yield: 69%, ¹H-NMR (400 MHz, CDCl₃) δ/ppm: 7.334-7.211 (d, 2H), 5.387-5.282 (m, 2H), 4.207 (s, 2H), 3.448-3.033 (m, 8H), 2.264-2.227 (t, 2H), 1.734-1.567 (m, 6H), 1.409 (s, 18H), 1.329-1.312 (d, 6H), 1.229 (bs, 20H), 0.870-0.836 (t, 3H). HRMS (m/z): 684.5241 [(M+H)⁺] (observed), 684.5275 [(M+H)⁺] (calculated).

### Example 1.14.1.3: N,N-bis-[{3-(Boc-^{L}Ala)amido}propyl]hexadecanamide (70c)

Yield: 68%, ¹H-NMR (400 MHz, CDCl₃) δ/ppm: 7.335-7.199 (d, 2H), 5.387-5.260 (m, 2H), 4.213-4.198 (d, 2H), 3.455-3.031 (m, 8H), 2.268-2.230 (t, 2H), 1.744-1.722 (m, 6H), 1.413 (s, 18H), 1.333-1.316 (d, 6H), 1.232 (bs, 24H), 0.875-0.840 (t, 3H). HRMS (m/z): 712.5558 [(M+H)⁺] (observed), 712.5558 [(M+H)⁺] (calculated).

### Example 1.14.2: N,N-bis-[{3-(^{L}Ala)amido}propyl]alkanamide bis(trifluoroacetate) (45, 46, 47)

About 1 equivalent of 70a-70c was dissolved in DCM (3 mL). To the intensely stirred solution 4 equivalents (excess amount) of trifluoroacetic acid (TFA) was added and allowed to stirring at RT for 12 h. Then solvent and unused TFA were removed to afford products with 100% yield.

### Example 1.14.2.1: N,N-bis-[{3-(^{L}Ala)amido}propyl]dodecanamide bis(trifluoroacetate) (45)

¹H-NMR (100 MHz, DMSO-d₆) δ/ppm: 8.417-8.084 (s, 8H), 3.793-3.776 (t, 2H), 3.247-3.037 (m, 8H), 2.284-2.212 (t, 2H), 1.660-1.472 (m, 6H), 1.347-1.333 (d, 6H), 1.237 (bs, 16H), 0.871-0.837 (t, 3H). HRMS (m/z): 456.3847 [(M+H)⁺] (observed), 456.3914 [(M+H)⁺] (calculated).

### Example 1.14.2.2: N,N-bis-[{3-(^{L}Ala)amido}propyl]tetradecanamide bis(trifluoroacetate) (46)

¹H-NMR (100 MHz, DMSO-d₆) δ/ppm: 8.531-8.130 (s, 8H), 3.817-3.785 (t, 2H), 3.243-3.035 (m, 8H), 2.244-2.209 (t, 2H), 1.675-1.466 (m, 6H), 1.346-1.333 (d, 6H), 1.232 (bs, 20H), 0.866-0.832 (t, 3H). HRMS (m/z): 484.4103 [(M+H)⁺] (observed), 484.4227 [(M+H)⁺] (calculated).

### Example 1.14.2.3: N,N-bis-[{3-(^{L}Ala)amido}propyl]hexadecanamide bis(trifluoroacetate) (47)

¹H-NMR (400 MHz, DMSO-d₆) δ/ppm: 8.460-8.071 (s, 8H), 3.534-3.527 (s, 4H), 3.333-3.070 (m, 8H), 2.252-2.218 (t, 2H), 1.657-1.595 (m, 6H), 1.235 (bs, 24H), 0.851-0.835 (t, 3H). HRMS (m/z): 512.4410 [(M+H)⁺] (observed), 512.4540 [(M+H)⁺] (calculated).

### Example 1.15.1: N,N-bis-[{3-(Boc-^{L}Val)amido}propyl]alkanamide (71a-70c)

About 2.4 equivalents of N-Boc-L-Valine was dissolved in dry DCM (12 mL) at 0° C. In the reaction mixture 6 equivalents of DIPEA was added followed by 2.4 equivalents of HBTU. Then DMF (3 mL) was added to the reaction mixture. After 10 minutes, 1 equivalent of 68a-68c in dry DCM (1 mL) was added drop wise. The reaction mixture was brought to RT and allowed to stir for 48 h. Solvent was evaporated and residue was diluted in ethyl acetate (50 mL). Then work-up was carried out at first with 1N HCl (3 times) followed by saturated Na₂CO₃ solution (3 times). The crude product was extracted in ethyl acetate layer. Finally purification was accomplished through column chromatography on silica gel (60-120 mesh) using methanol and chloroform as eluent to afford 71a-71c with 65-70% yield.

### Example 1.15.1.1: N,N-bis-[{3-(Boc-^{L}Val)amido}propyl]dodecanamide (71a)

Yield: 67%, ¹H-NMR (400 MHz, CDCl₃) δ/ppm: 7.553-7.486 (m, 2H), 5.425-5.271 (m, 2H), 3.949-3.910 (t, 2H), 3.606-3.900 (m, 8H), 2.238-2.184 (t, 2H), 1.916-1.541 (m, 8H), 1.386 (s, 18H), 1.207 (bs, 16H), 0.929-0.891 (m, 12H), 0.847-0.813 (t, 3H). HRMS (m/z): 712.5586 [(M+H)⁺] (observed), 712.5588 [(M+H)⁺] (calculated).

### Example 1.15.1.2: N,N-bis-[{3-(Boc-^{L}Val)amido}propyl]tetradecanamide (71b)

Yield: 66%, ¹H-NMR (400 MHz, CDCl₃) δ/ppm: 7.531-7.479 (m, 2H), 5.371-5.231 (m, 2H), 3.978-3.924 (t, 2H), 3.655-2.914 (m, 8H), 2.258-2.201 (t, 2H), 1.928-1.565 (m, 8H), 1.409 (s, 18H), 1.230 (bs, 20H), 0.953-0.913 (m, 12H), 0.872-0.838 (t, 3H). HRMS (m/z): 740.5850 [(M+H)⁺] (observed), 740.5901 [(M+H)⁺] (calculated).

### Example 1.15.1.3: N,N-bis-[{3-(Boc-^{L}Val)amido}propyl]hexadecanamide (71c)

Yield: 68%, ¹H-NMR (400 MHz, CDCl₃) δ/ppm: 7.515-7.475 (m, 2H), 5.366-5.224 (m, 2H), 3.977-3.924 (t, 2H), 3.649-2.907 (m, 8H), 2.257-2.219 (t, 2H), 1.928-1.563 (m, 8H), 1.407 (s, 18H), 1.228 (bs, 24H), 0.951-0.912 (m, 12H), 0.870-0.836 (t, 3H). HRMS (m/z): 768.6166 [(M+H)⁺] (observed), 768.6214 [(M+H)⁺] (calculated).

### Example 1.15.2: N,N-bis-[{3-(^{L}Val)amido}propyl]alkanamide bis(trifluoroacetate) (48, 49, 50)

About 1 equivalent of 71a-71c was dissolved in DCM (3 mL). To the intensely stirred solution 4 equivalents (excess amount) of trifluoroacetic acid (TFA) was added and allowed to stirring at RT for 12 h. Then solvent and unused TFA were removed to afford pure products with 100% yield.

### Example 1.15.2.1: N,N-bis-[{3-(Val)amido}propyl]dodecanamide bis(trifluoroacetate) (48)

¹H-NMR (100 MHz, DMSO-d₆) δ/ppm: 8.569-8.128 (m, 8H), 3.500 (s, 2H), 3.256-2.984 (m, 8H), 2.254-2.223 (t, 2H), 2.063-2.021 (m, 2H), 1.684-1.472 (m, 6H), 1.234 (bs, 16H), 0.936-0.919 (d, 12H), 0.868-0.834 (t, 3H). HRMS (m/z): 512.4481 [(M+H)⁺] (observed), 512.4540 [(M+H)⁺] (calculated).

### Example 1.15.2.2: N,N-bis-[{3-(^{L}Val)amido}propyl]tetradecanamide bis(trifluoroacetate) (49)

¹H-NMR (100 MHz, DMSO-d₆) δ/ppm: 8.569-8.122 (m, 8H), 3.520 (s, 2H), 3.256-2.984 (m, 8H), 2.254-2.223 (t, 2H), 2.063-2.019 (m, 2H), 1.685-1.472 (m, 6H), 1.234 (bs, 20H), 0.936-0.919 (d, 12H), 0.868-0.834 (t, 3H). HRMS (m/z): 540.4783 [(M+H)⁺] (observed), 540.4853 [(M+H)⁺] (calculated).

### Example 1.15.2.3: N,N-bis-[{3-(^{L}Val)amido}propyl]hexadecanamide bis(trifluoroacetate) (50)

¹H-NMR (100 MHz, DMSO-d₆) δ/ppm: 8.611-8.158 (m, 8H), 3.539 (s, 2H), 3.256-3.004 (m, 8H), 2.250-2.220 (t, 2H), 2.080-2.020 (m, 2H), 1.685-1.469 (m, 6H), 1.229 (bs, 24H), 0.936-0.919 (d, 12H), 0.864-0.830 (t, 3H). HRMS (m/z): 568.5128 [(M+H)⁺] (observed), 568.5166 [(M+H)⁺] (calculated).

### Example 1.16.1: N,N-bis-[{3-(Boc-^{L}Ile)amido}propyl]alkanamide (72a-72c)

About 2.4 equivalents of N-Boc-L-Isoleucine was dissolved in dry DCM (12 mL) at 0° C. In the reaction mixture 6 equivalents of DIPEA was added followed by 2.4 equivalents of HBTU. Then DMF (3 mL) was added to the reaction mixture. After 10 minutes, 1 equivalent of 68a-68c in dry DCM (1 mL) was added drop wise. The reaction mixture was brought to RT and allowed to stir for 48 h. Solvent was evaporated and residue was diluted in ethyl acetate (50 mL). Then work-up was carried out at first with 1N HCl (3 times) followed by saturated Na₂CO₃ solution (3 times). The crude product was extracted in ethyl acetate layer. Finally purification was accomplished through column chromatography on silica gel (60-120 mesh) using methanol and chloroform as eluent to afford 72a-72c with 65-70% yield.

### Example 1.16.1.1: N,N-bis-[{3-(Boc-^{L}Ile)amido}propyl]dodecanamide (72a)

Yield: 68%, ¹H-NMR (400 MHz, CDCl₃) δ/ppm: 7.530-7.469 (m, 2H), 5.315-5.183 (m, 2H), 3.998-3.917 (t, 2H), 3.672-2.887 (m, 8H), 2.254-2.209 (t, 2H), 2.002-1.557 (m, 10H), 1.403 (s, 18H), 1.227 (bs, 16H), 1.148-1.063 (m, 2H), 0.859-0.839 (m, 15H). HRMS (m/z): 740.5080 [(M+H)⁺] (observed), 740.5901 [(M+H)⁺] (calculated).

### Example 1.16.1.2: N,N-bis-[{3-(Boc-^{L}Ile)amido}propyl]tetradecanamide (72b)

Yield: 70%, ¹H-NMR (400 MHz, CDCl₃) δ/ppm: 7.532-7.572 (m, 2H), 5.317-5.185 (m, 2H), 4.013-3.959 (t, 2H), 3.673-2.891 (m, 8H), 2.259-2.204 (t, 2H), 2.008-1.567 (m, 10H), 1.410 (s, 18H), 1.235 (bs, 20H), 1.151-1.069 (m, 2H), 0.903-0.842 (m, 15H). HRMS (m/z): 768.6249 [(M+H)⁺] (observed), 768.6214 [(M+H)⁺] (calculated).

### Example 1.16.1.3: N,N-bis-[{3-(Boc-^{L}Ile)amido}propyl]hexadecanamide (72c)

Yield: 67%, ¹H-NMR (400 MHz, CDCl₃) δ/ppm: 7.581-7.492 (m, 2H), 5.435-5.279 (m, 2H), 3.972-3.929 (t, 2H), 3.564-2.892 (m, 8H), 2.225-2.174 (t, 2H), 1.794-1.529 (m, 10H), 1.371 (s, 18H), 1.197 (bs, 24H), 1.113-1.021 (m, 2H), 0.863-0.803 (m, 15H). HRMS (m/z): 796.6477 [(M+H)⁺] (observed), 796.6527 [(M+H)⁺] (calculated).

### Example 1.16.2: N,N-bis-[{3-(^{L}Ile)amido}propyl]alkanamide bis(trifluoroacetate) (51, 52, 52)

About 1 equivalent of 72a-72c was dissolved in DCM (3 mL). To the intensely stirred solution 4 equivalents (excess amount) of trifluoroacetic acid (TFA) was added and allowed to stirring at RT for 12 h. Then solvent and unused TFA were removed to afford pure products with 100% yield.

### Example 1.16.2.1: N,N-bis-[{3-(^{L}Ile)amido}propyl]dodecanamide bis(trifluoroacetate) (51)

¹H-NMR (100 MHz, DMSO-d₆) δ/ppm: 8.518-8.111 (m, 8H), 3.562 (s, 2H), 3.333-2.976 (m, 8H), 2.251-2.224 (t, 2H), 1.794-1.471 (m, 10H), 1.232 (bs, 16H), 1.152-1.076 (m, 2H), 0.901-0.832 (m, 15H). HRMS (m/z): 540.4715 [(M+H)^{+]} (observed), 540.4853 [(M+H)⁺] (calculated).

### Example 1.16.2.2: N,N-bis-[{3-(^{L}Ile)amido}propyl]tetradecanamide bis(trifluoroacetate) (52)

¹H-NMR (100 MHz, DMSO-d₆) δ/ppm: 8.527-8.116 (m, 8H), 3.566 (s, 2H), 3.337-2.978 (m, 8H), 2.257-2.227 (t, 2H), 1.796-1.479 (m, 10H), 1.234 (bs, 20H), 1.156-1.081 (m, 2H), 0.905-0.835 (m, 15H). HRMS (m/z): 568.5097 [(M+H)^{+]} (observed), 568.5166 [(M+H)⁺] (calculated).

### Example 1.16.2.3: N,N-bis-[{3-(^{L}Ile)amido}propyl]hexadecanamide bis(trifluoroacetate) (53)

¹H-NMR (100 MHz, DMSO-d₆) δ/ppm: 8.572-8.146 (m, 8H), 3.570 (s, 2H), 3.254-2.963 (m, 8H), 2.262-2.219 (t, 2H), 1.797-1.456 (m, 10H), 1.231 (bs, 24H), 1.154-1.079 (m, 2H), 0.902-0.832 (m, 15H). HRMS (m/z): 596.5325 [(M+H)^{+]} (observed), 596.5479 [(M+H)⁺] (calculated).

### Example 1.17.1: N,N-bis-[{3-(Boc-^{L}Leu)amido}propyl]alkanamide (73a-73c)

About 2.4 equivalents of N-Boc-L-Leucine was dissolved in dry DCM (12 mL) at 0° C. In the reaction mixture 6 equivalents of DIPEA was added followed by 2.4 equivalents of HBTU. Then DMF (3 mL) was added to the reaction mixture. After 10 minutes, 1 equivalent of 68a-68c in dry DCM (1 mL) was added drop wise. The reaction mixture was brought to RT and allowed to stir for 48 h. Solvent was evaporated and residue was diluted in ethyl acetate (50 mL). Then work-up was carried out at first with 1N HCl (50 mL, 3 times) followed by saturated Na₂CO₃ solution (50 mL, 3 times). The crude product was extracted in ethyl acetate layer. Finally purification was accomplished through column chromatography on silica gel (60-120 mesh) using methanol and chloroform as eluent to afford 73a-73c with 65-70% yield.

### Example 1.17.1.1: N,N-bis-[{3-(Boc-^{L}Leu)amido}propyl]dodecanamide (73a)

Yield: 69%, ¹H-NMR (400 MHz, CDCl₃) δ/ppm: 7.637-7.534 (s, 2H), 5.246-5.126 (d, 2H), 4.246-4.126 (t, 2H), 3.628-2.890 (m, 8H), 2.254-2.180 (t, 2H), 2.160-2.135 (m, 2H), 1.719-1.458 (m, 10H), 1.400 (s, 18H), 1.230 (bs, 16H), 0.923-0.835 (m, 15H). HRMS (m/z): 740.5845 [(M+H)⁺] (observed), 740.5901 [(M+H)⁺] (calculated).

### Example 1.17.1.2: N,N-bis-[{3-(Boc-LLeu)amido}propyl]tetradecanamide (73b)

Yield: 66%, ¹H-NMR (400 MHz, CDCl₃) δ/ppm: 7.621-7.527 (s, 2H), 5.248-5.129 (d, 2H), 4.248-4.129 (t, 2H), 3.480-2.893 (m, 8H), 2.257-2.220 (t, 2H), 2.160-1.458 (m, 12H), 1.404 (s, 18H), 1.232 (bs, 20H), 0.912-0.839 (m, 15H). HRMS (m/z): 768.6181 [(M+H)⁺] (observed), 768.6214 [(M+H)⁺] (calculated).

### Example 1.17.1.3: N,N-bis-[{3-(Boc-^{L}Leu)amido}propyl]hexadecanamide (73c)

Yield: 68%, ¹H-NMR (400 MHz, CDCl₃) δ/ppm: 7.624-7.530 (s, 2H), 5.252-5.132 (d, 2H), 4.219-4.201 (t, 2H), 3.634-2.894 (m, 8H), 2.257-2.220 (t, 2H), 1.746-1.454 (m, 12H), 1.404 (s, 18H), 1.233 (bs, 24H), 0.912-0.840 (m, 15H). HRMS (m/z): 796.6482 [(M+H)⁺] (observed), 796.6527 [(M+H)⁺] (calculated).

### Example 1.17.2: N,N-bis-[{3-(^{L}Leu)amido}propyl]alkanamide bis(trifluoroacetate) (54, 55, 56)

About 1 equivalent of 70a-70c was dissolved in DCM (3 mL). To the intensely stirred solution 4 equivalents (excess amount) of trifluoroacetic acid (TFA) was added and allowed to stirring at RT for 12 h. Then solvent and unused TFA were removed to afford pure products with 100% yield.

### Example 1.17.2.1: N,N-bis-[{3-(^{L}Leu)amido}propyl]dodecanamide bis(trifluoroacetate) (54)

¹H-NMR (400 MHz, DMSO-d₆) δ/ppm: 8.670-8.176 (m, 8H), 3.758-3.715 (t, 2H), 3.252-3.007 (m, 8H), 2.240-2.227 (t, 2H), 1.684-1.476 (m, 12H), 1.234 (bs, 16H), 0.906-0.833 (m, 15H). HRMS (m/z): 540.4860 [(M+H)⁺] (observed), 540.4853 [(M+H)⁺] (calculated).

### Example 1.17.2.2: N,N-bis-[{3-(^{L}Leu)amido}propyl]tetradecanamide bis(trifluoroacetate) (55)

¹H-NMR (400 MHz, DMSO-d₆) δ/ppm: 8.523-8.188 (m, 8H), 3.710 (s, 2H), 3.253-2.994 (m, 8H), 2.257-2.225 (t, 2H), 1.667-1.474 (m, 12H), 1.231 (bs, 20H), 0.904-0.830 (m, 15H). HRMS (m/z): 568.5182 [(M+H)⁺] (observed), 568.5166 [(M+H)^{+]} (calculated).

### Example 1.17.2.3: N,N-bis-[{3-(^{L}Leu)amido}propyl]hexadecanamide bis(trifluoroacetate) (56)

¹H-NMR (400 MHz, DMSO-d₆) δ/ppm: 8.608-8.130 (m, 8H), 3.694 (s, 2H), 3.238-2.994 (m, 8H), 2.240-2.227 (t, 2H), 1.683-1.477 (m, 12H), 1.234 (bs, 24H), 0.911-0.853 (m, 15H). HRMS (m/z): 596.5475 [(M+H)⁺] (observed), 596.5479 [(M+H)^{+]} (calculated).

### Example 1.18.1: N,N-bis-[{3-(Boc-^{L}Pro)amido}propyl]alkanamide (74a-74c)

About 2.4 equivalents of N-Boc-L-Proline was dissolved in dry DCM (12 mL) at 0° C. In the reaction mixture 6 equivalents of DIPEA was added followed by 2.4 equivalents of HBTU. Then DMF (3 mL) was added to the reaction mixture. After 10 minutes, 1 equivalent of 68a-68c in dry DCM (1 mL) was added drop wise. The reaction mixture was brought to RT and allowed to stir for 48 h. Solvent was evaporated and residue was diluted in ethyl acetate (50 mL). Then work-up was carried out at first with 1N HCl (3 times) followed by saturated Na₂CO₃ solution (3 times). The crude product was extracted in ethyl acetate layer. Finally purification was accomplished through column chromatography on silica gel (60-120 mesh) using methanol and chloroform as eluent to afford 74a-74c with 65-70% yield.

### Example 1.18.1.1: N,N-bis-[{3-(Boc-^{L}Pro)amido}propyl]dodecanamide (74a)

Yield: 70%, ¹H-NMR (400 MHz, CDCl₃) δ/ppm: 7.441-6.795 (s, 2H), 4.219-4.131 (t, 2H), 3.532-3.055 (m, 12H), 2.265-2.228 (t, 2H), 2.164-1.583 (m, 14H), 1.429 (s, 18H), 1.229 (bs, 16H), 0.869-0.835 (t, 3H). HRMS (m/z): 708.5245 [(M+H)⁺] (observed), 708.5275 [(M+H)⁺] (calculated).

### Example 1.18.1.2: N,N-bis-[{3-(Boc-^{L}Pro)amido}propyl]tetradecanamide (74b)

Yield: 65%, ¹H-NMR (400 MHz, CDCl₃) δ/ppm: 7.509-6.795 (s, 2H), 4.219-4.131 (t, 2H), 3.553-3.055 (m, 12H), 2.265-2.164 (t, 2H), 2.164-1.583 (m, 14H), 1.420 (s, 18H), 1.229 (bs, 20H), 0.869-0.835 (t, 3H). HRMS (m/z): 736.5596 [(M+H)⁺] (observed), 736.5588 [(M+H)⁺] (calculated).

### Example 1.18.1.3: N,N-bis-[{3-(Boc-^{L}Pro)amido}propyl]hexadecanamide (74c)

Yield: 68%, ¹H-NMR (400 MHz, CDCl₃) δ/ppm: 7.519-6.806 (s, 2H), 4.224-4.136 (t, 2H), 3.558-3.088 (m, 12H), 2.269-2.232 (t, 2H), 2.152-1.586 (m, 14H), 1.425 (s, 18H), 1.232 (bs, 16H), 0.874-0.840 (t, 3H). HRMS (m/z): 764.5910 [(M+H)⁺] (observed), 764.5901 [(M+H)⁺] (calculated).

### Example 1.18.2: N,N-bis-[{3-(^{L}Pro)amido}propyl]alkanamide bis(trifluoroacetate) (57, 58, 59)

About 1 equivalent of 74a-74c was dissolved in DCM (3 mL). To the intensely stirred solution 4 equivalents (excess amount) of trifluoroacetic acid (TFA) was added and allowed to stirring at RT for 12 h. Then solvent and unused TFA were removed to afford pure products with 100% yield.

### Example 1.18.2.1: N,N-bis-[{3-(^{L}Pro)amido}propyl]dodecanamide bis(trifluoroacetate) (57)

¹H-NMR (400 MHz, DMSO-d₆) δ/ppm: 9.787 (s, 2H), 8.731-8.540 (m, 4H) 4.168-4.151 (m, 2H), 3.227-2.885 (m, 12H), 2.280-1.464 (m, 16H), 1.230 (bs, 16H), 0.862-0.828 (t, 3H). HRMS (m/z): 508.3608 [(M+H)⁺] (observed), 508.4227 [(M+H)⁺] (calculated).

### Example 1.18.2.2: N,N-bis-[{3-(^{L}Pro)amido}propyl]tetradecanamide bis(trifluoroacetate) (58)

¹H-NMR (400 MHz, DMSO-d₆) δ/ppm: 9.683 (s, 2H), 8.702-8.518 (m, 4H) 4.157-4.144 (m, 2H), 3.302-3.009 (m, 12H), 2.500-1.465 (m, 16H), 1.230 (bs, 20H), 0.862-0.828 (t, 3H). HRMS (m/z): 536.3861 [(M+H)⁺] (observed), 536.4540 [(M+H)⁺] (calculated).

### Example 1.18.2.3: N,N-bis-[{3-(^{L}Pro)amido}propyl]hexadecanamide bis(trifluoroacetate) (59)

¹H-NMR (400 MHz, DMSO-d₆) δ/ppm: 9.715 (s, 2H), 8.713-8.525 (m, 4H) 4.163-4.146 (m, 2H), 3.231-2.994 (m, 12H), 2.500-1.465 (m, 16H), 1.229 (bs, 24H), 0.847-0.829 (t, 3H). HRMS (m/z): 564.4152 [(M+H)⁺] (observed), 564.4853 [(M+H)⁺] (calculated).

### Example 1.19.1: N¹-(Boc-^{D}Leu)-N³-[{3-(Boc-^{D}Leu)amido}propyl]propane-1,3-diamine (75)

About 2 equivalents of N-Boc-D-Leucine was dissolved in about 25 mL of dry DCM at 0° C. In the reaction mixture about 6 equivalents of DIPEA was added followed by about 2 equivalents of HBTU. Now about 8 mL of DMF was added to the reaction mixture. After 10 minutes, about 1 equivalent of norspermidine [Bis (3-aminopropyl) amine] was added drop wise. The reaction mixture was allowed to stir for 48 h at 0° C. Then reaction solvent was evaporated and residue was diluted in ethyl acetate. Thereafter work-up was carried out with 1N HCl (3 times) followed by saturated Na₂CO₃ solution (3 times). The crude product was collected in ethyl acetate layer. Finally column was done to isolate the product with 67% yield. HRMS (m/z): 558.4241 [(M+H)+] (observed), 558.4231 [(M+H)+] (calculated).

### Example 1.19.2: N,N-Bis[3-(Boc-^{D}Leu)amidopropyl]octanamide (76)

About 1.5 equivalents of octanoic was dissolved in dry DCM (12 mL) at 0° C. To the reaction mixture about 4 equivalents of DIPEA was then added followed by 1.5 equivalents of HBTU. Then DMF (3 mL) was added to the reaction mixture. After 10 min, 1 equivalent of 75 in dry DCM (2 mL) was added dropwise. The reaction mixture was brought to room temperature and allowed to stir for 24 h. At the end of 24 h, solvent was evaporated and residue was diluted in ethyl acetate. The reaction mixture was washed at first with 1 N HCl (3 times) followed by saturated Na₂CO₃ solution (3 times). The crude product was extracted in ethyl acetate layer and dried over anhydrous sodium sulfate, and finally purification was accomplished through column chromatography on silica gel (60-120 mesh) using different percentage of methanol and chloroform as eluent to afford 76 with 72% yield. HRMS (m/z): 684.5204 [(M+H)⁺] (observed), 684.5275 [(M+H)+](calculated).

### Example 1.19.3: N,N-Bis[3-(^{D}Leu)amidopropyl]octanamide bis(trifluoroacetate) (77)

About 1 equivalent of 76 was dissolved in DCM (3 mL). To the intensely stirred solution 4 equivalents (excess amount) of trifluoroacetic acid (TFA) was added and allowed to stirring at RT for 12 h. Then solvent and unused TFA were removed to afford pure products with 100% yield. HRMS (m/z): 484.4184 [(M+H)+] (observed), 484.4227 [(M+H)+] (calculated).

### Example 1.19.4: N,N-Bis[3-(Boc-^{D}AA^{D}Leu)amidopropyl]octanamide (78a-78c)

About 2 equivalents of N,N-Di-Boc-D-Lysine, N-Boc-D-Leucine or N-Boc-D-Phenylalanine was dissolved in about 25 mL of dry DCM at 0° C. In the reaction mixture about 6 equivalents of DIPEA was added followed by about 2 equivalents of HBTU. Now about 8 mL of DMF was added to the reaction mixture. After 10 min, 1 equivalent of 77 in dry DCM (2 mL) was added dropwise. The reaction mixture was brought to room temperature and allowed to stir for 48 h. At the end of 48 h, solvent was evaporated and residue was diluted in ethyl acetate. The reaction mixture was washed at first with 1 N HCl (3 times) followed by saturated Na₂CO₃ solution (3 times). The crude product was extracted in ethyl acetate layer and dried over anhydrous sodium sulfate, and finally purification was accomplished through column chromatography on silica gel (60-120 mesh) using different percentage of methanol and chloroform as eluent to afford 78a-78c with 65-70% yield.

### Example 1.19.4.1: N,N-Bis[3-(Boc-^{D}Lys-Boc^{D}Leu)amidopropyl]octanamide (78a)

Yield-68%, HRMS (m/z): 1140.8053 [(M+H)⁺] (observed), 1140.8223 [(M+H)+](calculated).

### Example 1.19.4.2: N,N-Bis[3-(Boc-^{D}Leu^{D}Leu)amidopropyl]octanamide (78b)

Yield-66%, HRMS (m/z): 910.6837 [(M+H)⁺] (observed), 910.6957 [(M+H)⁺] (calculated).

### Example 1.19.4.3: N,N-Bis[3-(Boc-^{D}Phe^{D}Leu)amidopropyl]octanamide (78c)

Yield-68%, HRMS (m/z): 978.6502 [(M+H)⁺] (observed), 978.6644 [(M+H)⁺] (calculated).

### Example 1.19.5: N,N-Bis[3-(^{D}AA^{D}Leu)amidopropyl]octanamide bis/tetrakis(trifluoroacetate) (60, 61, 62)

About 1 equivalent of 78a-78c was dissolved in DCM (3 mL). To the intensely stirred solution 4 equivalents (excess amount) of trifluoroacetic acid (TFA) was added and allowed to stirring at RT for 12 h. Then solvent and unused TFA were removed to afford pure products with 100% yield.

### Example 1.19.5.1: N,N-Bis[3-(^{D}Lys^{D}Leu)amidopropyl]octanamide tetrakis(trifluoroacetate) (60)

HRMS (m/z): 740.5940 [(M+H)⁺] (observed), 740.6126 [(M+H)⁺] (calculated).

### Example 1.19.5.2: N,N-Bis[3-(^{D}Leu^{D}Leu)amidopropyl]octanamide bis(trifluoroacetate) (61)

HRMS (m/z): 710.5724 [(M+H)⁺] (observed), 710.5908 [(M+H)⁺] (calculated).

### Example 1.19.5.3: N,N-Bis[3-(^{D}Phe^{D}Leu)amidopropyl]octanamide bis(trifluoroacetate) (62)

HRMS (m/z): 778.5375 [(M+H)⁺] (observed), 778.5595 [(M+H)⁺] (calculated).

### Example 1.20.1: N¹-(Boc-^{D}Phe)-N³-[{3-(Boc-^{D}Phe)amido}propyl]propane-1,3-diamine (79)

About 2 equivalents of N-Boc-D-Phenylalanine was dissolved in about 25 mL of dry DCM at 0° C. In the reaction mixture about 6 equivalents of DIPEA was added followed by about 2 equivalents of HBTU. Now about 8 mL of DMF was added to the reaction mixture. After 10 minutes, about 1 equivalent of norspermidine [Bis (3-aminopropyl) amine] was added drop wise. The reaction mixture was allowed to stir for 48 h at 0° C. Then reaction solvent was evaporated and residue was diluted in ethyl acetate. Thereafter work-up was carried out with 1N HCl (3 times) followed by saturated Na₂CO₃ solution (3 times). The crude product was collected in ethyl acetate layer. Finally column was done to isolate the product with 70% yield. HRMS (m/z): 626.3898 [(M+H)+] (observed), 626.3918 [(M+H)⁺] (calculated).

### Example 1.20.2: N,N-Bis[3-(Boc-^{D}Phe)amidopropyl]octanamide (80)

About 1.5 equivalents of octanoic was dissolved in dry DCM (12 mL) at 0° C. To the reaction mixture about 4 equivalents of DIPEA was then added followed by 1.5 equivalents of HBTU. Then DMF (3 mL) was added to the reaction mixture. After 10 min, 1 equivalent of 79 in dry DCM (2 mL) was added dropwise. The reaction mixture was brought to room temperature and allowed to stir for 24 h. At the end of 24 h, solvent was evaporated and residue was diluted in ethyl acetate. The reaction mixture was washed at first with 1 N HCl (3 times) followed by saturated Na₂CO₃ solution (3 times). The crude product was extracted in ethyl acetate layer and dried over anhydrous sodium sulfate, and finally purification was accomplished through column chromatography on silica gel (60-120 mesh) using different percentage of methanol and chloroform as eluent to afford 80 with 74% yield. HRMS (m/z): 752.4911 [(M+H)⁺] (observed), 752.4962 [(M+H)+](calculated).

### Example 1.20.3: N,N-Bis[3-(^{D}Phe)amidopropyl]octanamide bis(trifluoroacetate) (81)

About 1 equivalent of 80 was dissolved in DCM (3 mL). To the intensely stirred solution 4 equivalents (excess amount) of trifluoroacetic acid (TFA) was added and allowed to stirring at RT for 12 h. Then solvent and unused TFA were removed to afford pure products with 100% yield. HRMS (m/z): 552.3946 [(M+H)+] (observed), 552.3914 [(M+H)+] (calculated).

### Example 1.20.4: N,N-Bis[3-(Boc-^{D}AA^{D}Phe)amidopropyl]octanamide (82a-82c)

About 2 equivalents of N,N-Di-Boc-D-Lysine, N-Boc-D-Leucine or N-Boc-D-phenylalanine was dissolved in about 25 mL of dry DCM at 0° C. In the reaction mixture about 6 equivalents of DIPEA was added followed by about 2 equivalents of HBTU. Now about 8 mL of DMF was added to the reaction mixture. After 10 min, 1 equivalent of 81 in dry DCM (2 mL) was added dropwise. The reaction mixture was brought to room temperature and allowed to stir for 48 h. At the end of 48 h, solvent was evaporated and residue was diluted in ethyl acetate. The reaction mixture was washed at first with 1 N HCl (3 times) followed by saturated Na₂CO₃ solution (3 times). The crude product was extracted in ethyl acetate layer and dried over anhydrous sodium sulfate, and finally purification was accomplished through column chromatography on silica gel (60-120 mesh) using different percentage of methanol and chloroform as eluent to afford 82a-82c with 65-70% yield.

### Example 1.20.4.1: N,N-Bis[3-(Boc-^{D}Lys-Boc^{D}Phe)amidopropyl]octanamide (82a)

Yield-67%, HRMS (m/z): 1208.7910 [(M+H)⁺] (observed), 1208.7910 [(M+H)+](calculated).

### Example 1.20.4.2: N,N-Bis[3-(Boc-^{D}Leu^{D}Phe)amidopropyl]octanamide (82b)

Yield-68%, HRMS (m/z): 978.6630 [(M+H)⁺] (observed), 978.6644 [(M+H)⁺] (calculated).

### Example 1.20.4.3: N,N-Bis[3-(Boc-^{D}Phe^{D}Phe)amidopropyl]octanamide (82c)

Yield-65%, HRMS (m/z): 1064.6280 [(M+H)⁺] (observed), 1046.6331 [(M+H)⁺] (calculated).

### Example 1.20.5: N,N-Bis[3-(^{D}AA^{D}Phe)amidopropyl]octanamide bis/tetrakis(trifluoroacetate) (63, 64, 65)

About 1 equivalent of 82a-82c was dissolved in DCM (3 mL). To the intensely stirred solution 4 equivalents (excess amount) of trifluoroacetic acid (TFA) was added and allowed to stirring at RT for 12 h. Then solvent and unused TFA were removed to afford pure products with 100% yield.

### Example 1.20.5.1: N,N-Bis[3-(^{D}Lys^{D}Phe)amidopropyl]octanamide tetrakis(trifluoroacetate) (63)

HRMS (m/z): 808.5796 [(M+H)⁺] (observed), 808.5813 [(M+H)⁺] (calculated).

### Example 1.20.5.2: N,N-Bis[3-(^{D}Leu^{D}Phe)amidopropyl]octanamide bis(trifluoroacetate) (64)

HRMS (m/z): 778.5390 [(M+H)⁺] (observed), 778.5595 [(M+H)⁺] (calculated).

### Example 1.20.5.3: N,N-Bis[3-(^{D}Phe^{D}Phe)amidopropyl]octanamide bis(trifluoroacetate) (65)

HRMS (m/z): 846.5059 [(M+H)⁺] (observed), 846.5282 [(M+H)⁺] (calculated).

### Example 2

### In-Vitro Antibacterial Activity

Minimum Inhibitory Concentration (MIC): Antibacterial activity is reported as Minimum Inhibitory Concentration (MIC), which is the lowest concentration of the antibacterial agent that able to inhibit the growth of microorganism after overnight incubation. All synthesized compounds were assayed in a modified micro-dilution broth format. Stock solutions were made by serially diluting the compounds using autoclaved millipore water.

The bacterial freeze dried stock samples were stored at −80° C. About 5 μL of these stocks were added to about 3 mL of the respective broth and the culture was grown for about 6 h at about 37° C. with prior to the experiments. This 6 h grown culture gives about 10⁹ cfu/mL in case of S. aureus (MTCC 737), MRSA (ATCC 33591), and 10⁸ cfu/mL in case of E. coli (MTCC 443), Enterococcus faecium (ATCC 19634), VRE and Klebsiella pneumonie (ATCC 700603) which were determined by spread plating method. This 6 h grown culture was diluted to give effective cell concentration of 10⁵ cfu/mL, was then used for determining MIC. Compounds were serially diluted, in sterile millipore water (as 2 fold manner) in a way, so that the maximum working concentration was 250 μg/mL. About 50 μL of these serial dilutions were added to the wells of 96 well plate followed by the addition of about 150 μL of bacterial solution. The plates were then incubated at about 37° C., 150 rpm in the incubator and O.D at 600 nm was recorded at 24 h using TECAN (Infinite series, M200 pro) Plate Reader. Each concentration had triplicate values and the whole experiment was done at least twice and the MIC value was determined by taking the average of triplicate O. D. values for each concentration and plotting it against concentration. The data was then subjected to sigmoidal fitting. From the curve the MIC value was determined, as the point in the curve where the O.D. was similar to that of control having no bacteria.

The antimicrobial activities of these compounds were determined against a variety of bacteria by evaluating their Minimum Inhibition Concentrations (MIC). Most of the compounds were active against both gram-positive and gram-negative bacteria at micro molar concentrations which is comparable to the clinically approved conventional antibiotics. For example, compound 2, 6, 7, 12 and 17 were active against Staphylococcus aureus at a concentration of range 2-5 μM (Table 1). Similarly compound 11 and 12 showed very good activity with MIC values 2 and 1.1 μM respectively against Enterococcus faecium. Other compounds also showed high activity against this bacteria, like compound 2, 6, 7, 13, 15, 16 and 17 have the MIC value in the range of 2-5 μM (Table 1). In case of E. coli only lysine and ornithine series of compounds have shown good activity, compounds 11, 12, 15, 16 and 17 having MIC value in the concentration range of 4-6 μM (Table 1). Vancomycin resistant Enterococcus faecium (VRE) is a multi-drug resistant bacterium, which is highly pathogenic. All the compounds were active against VRE and most of the compound having MIC value below 5 μM (Table 2), the best activity with MIC values 1 and 1.5 μM were shown by the compounds 11 and 12 respectively. Most of the compound was active against MRSA and multi-drug resistant Klebsiella pneumonie in the range of micro molar (3-10 μM) concentration (Table 2).

All the antibacterial activities have been furnished in Tables: 1 and 2.

TABLE 1
Antibacterial activities of the compounds against wild-type
Gram-positive and Gram-negative bacteria.

| | Minimum Inhibitory Concentration | | |
|---|---|---|---|
| | (μM) | | |
| Compounds | S. aureus | E. faceium | E. coli |
| 1 | 19 | 36 | 24 |
| 2 | 4.3 | 4.8 | 12 |
| 3 | 7 | 5.2 | 16 |
| 4 | 10 | 8.5 | 225 |
| 5 | 12 | 11 | 270 |
| 6 | 4.2 | 3.3 | 12 |
| 7 | 2.5 | 2.5 | 39 |
| 8 | 6.5 | 5.4 | >257 |
| 9 | 53 | 60 | 40 |
| 10 | 37 | 32 | 14 |
| 11 | 7.8 | 2 | 5.9 |
| 12 | 4.7 | 1.1 | 5.4 |
| 13 | 7.7 | 4.5 | 8.4 |
| 14 | 7.9 | 5.2 | 6.6 |
| 15 | 6.4 | 3.2 | 5.2 |
| 16 | 5.7 | 4.5 | 5.4 |
| 17 | 3.9 | 2.7 | 4.8 |
| 18 | 38 | ND | 68 |
| 19 | 313 | ND | 343 |
| 20 | 86 | ND | 133 |
| 21 | >304 | >304 | >304 |
| 22 | >250 | >250 | >250 |
| 23 | >250 | >250 | >250 |

ND stands for “not determined”.

TABLE 2
Antibacterial activities of the compounds against drug-resistant
Gram-positive and Gram-negative bacteria.

| | Minimum Inhibitory Concentration | | |
|---|---|---|---|
| | (μM) | | |
| Compounds | MRSA | VRE | K. pneumonie |
| 1 | 25 | 17 | 70 |
| 2 | 4 | 2.7 | 9.7 |
| 3 | 4.8 | 2.8 | 18 |
| 4 | 5.2 | 3 | 70 |
| 5 | 5.6 | 3.2 | 95 |
| 6 | ND | 2.9 | ND |
| 7 | ND | 2.7 | ND |
| 8 | ND | 7 | ND |
| 9 | 65 | 58 | 98 |
| 10 | 45 | 30 | 40 |
| 11 | 14 | 1.5 | 9.3 |
| 12 | 4.8 | 1 | 6.5 |
| 13 | 14 | 2.2 | 7.5 |
| 14 | ND | 5.3 | ND |
| 15 | ND | 3 | ND |
| 16 | ND | 2.8 | ND |
| 17 | ND | 2.2 | ND |
| 18 | ND | ND | ND |
| 19 | ND | ND | ND |
| 20 | ND | ND | ND |
| 21 | ND | >304 | ND |
| 22 | >250 | >250 | >250 |
| 23 | >250 | >250 | >250 |

ND stands for “not determined”.

### Example 3

### Haemolytic Activity

Erythrocytes were isolated from freshly drawn, heparanized human blood and re-suspended to 5 vol % in PBS (pH 7.4). In a 96-well microtiter plate, 150 μL of erythrocyte suspension was added to 50 μL of serially diluted compound. Two controls were made, one without compound and other with 50 μL of 1 vol % solution of Triton X-100. The plate was incubated for 1 h at 37° C. The plate was then centrifuged at 3,500 rpm for 5 min, 100 μL of the supernatant from each well was transferred to a fresh 96-well microtiter plate, and A₅₄₀ was measured. Percentage of hemolysis was determined as (A−A₀)/(Aₜₒₜₐₗ−A₀)×100, where A is the absorbance of the test well, A₀ the absorbance of the negative controls (without compound), and Aₜₒₜₐₗ the absorbance of 100% hemolysis wells (with Triton X-100), all at 540 nm.

Toxicity studies of the compounds were done on freshly drawn human RBCs. Toxicity of the compounds was defined by their HC₅₀ values (Table 3), i.e. the concentration of compound at which 50% of the blood cells are lysed. Hemolytic studies showed that all of these compounds were selective towards bacteria over human RBCs. Compound 9 and 10 did not show any hemolytic even at 1000 M concentration also. But, at the same time these compounds are not good from activity point of view. The best compound from selectivity (HC₅₀/MIC) point of view is compound 11, which shows 50% hemolysis at 588 M having selectivity of 392 with respect to resistant bacteria VRE.

TABLE 3
Hemolytic activitie of the compounds against freshly
drawn human Red Blood Cells.

| Compounds | HC₅₀ |
|---|---|
| 1 | 232 |
| 2 | 205 |
| 3 | 144 |
| 4 | 83 |
| 5 | 71 |
| 6 | ND |
| 7 | ND |
| 8 | ND |
| 9 | >1000 |
| 10 | >1000 |
| 11 | 588 |
| 12 | 264 |
| 13 | 108 |
| 14 | ND |
| 15 | ND |
| 16 | ND |
| 17 | ND |
| 18 | ND |
| 19 | ND |
| 20 | ND |
| 21 | ND |
| 22 | ND |
| 23 | ND |

### Example 4

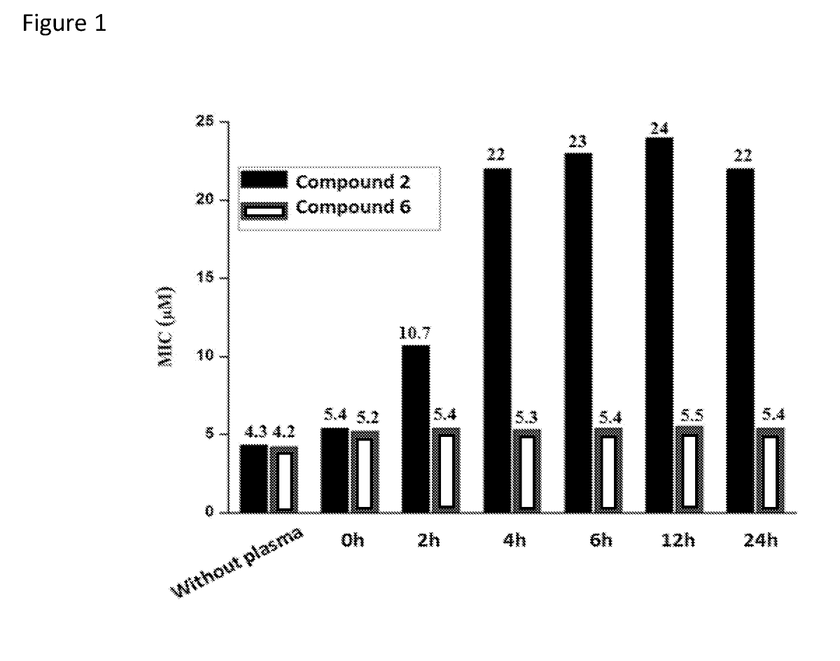

Antibacterial efficacy in human plasma: One of the limitations of lipopeptides is liability towards protease degradation. We were able to overcome this limitation through our synthetic design. We have incorporated D-Phenylalanine (compound 6) in place of L-Phenylalanine (compound 2) and performed the MIC experiment at different time intervals after incubating the compounds in 50% human plasma (FIG. 1). We found that MIC values increased from 5.4 μM to 10.7 μM after 2 h incubation of compound 2 with 50% plasma whereas it increased further to 22 μM after 4 h incubation, then MIC values remained same up to 24 h incubation. But, Compound 6 did not show any loss of activity in the plasma even up to 24 h incubation. It implied that compound 6 is stable in plasma.

TABLE 4
Antibacterial activities of the compounds (42-59) against wild-type
Gram-positive and Gram-negative bacteria.

| | Minimum Inhibitory Concentration | | |
|---|---|---|---|
| | (μM) | | |
| Compounds | S. aureus | E. faceium | E. coli |
| 42 | 67 | 86 | 340 |
| 43 | 12.5 | 8.8 | 16 |
| 44 | 6.5 | 4 | 5.9 |
| 45 | 95 | 73 | 282 |
| 46 | 19 | 9 | 17 |
| 47 | 5.6 | 3 | 4 |
| 48 | 28 | 25 | 18 |
| 49 | 4 | 3.6 | 7 |
| 50 | 2.7 | 1.7 | 10 |
| 51 | 8 | 8 | 11 |
| 52 | 3 | 4 | 5 |
| 53 | 4 | 3 | 26 |
| 54 | 5.8 | 8.6 | 8.8 |
| 55 | 3.6 | 4.2 | 5.4 |
| 56 | 3.8 | 3.4 | 20 |
| 57 | 60 | 78 | 90 |
| 58 | 11 | 13 | 12 |
| 59 | 2.8 | 4 | 4.4 |

TABLE 5
Antibacterial activities of the compounds (42-59)
against drug-resistant Gram-positive and Gram-negative bacteria.

| | Minimum Inhibitory Concentration | | |
|---|---|---|---|
| | (μM) | | |
| Compounds | MRSA | VRE | K. pneumonie |
| 42 | 61 | 112 | >340 |
| 43 | 10.8 | 13 | >365 |
| 44 | 10 | 3.3 | >365 |
| 45 | 133 | 82 | >365 |
| 46 | 17 | 7.6 | >360 |
| 47 | 6 | 3 | 12 |
| 48 | 40 | 25 | 58 |
| 49 | 3.4 | 4 | 7.3 |
| 50 | 1.9 | 1.4 | >314 |
| 51 | 5.7 | 11.3 | 28 |
| 52 | 2.9 | 2.6 | 300 |
| 53 | 2.6 | 4.2 | >300 |
| 54 | 8.9 | 5 | 18 |
| 53 | 2.7 | 2.6 | 110 |
| 56 | 2.9 | 3.4 | 280 |
| 57 | 130 | 112 | >340 |
| 58 | 15 | 18 | 135 |
| 59 | 7.2 | 2.7 | 12 |

TABLE 6
Hemolytic activities of the compounds (42-59) against freshly
drawn human Red Blood Cells

| Compounds | HC₅₀ (μM) |
|---|---|
| 42 | 400 |
| 43 | 157 |
| 44 | 90 |
| 45 | 562 |
| 46 | 93 |
| 47 | 68 |
| 48 | 264 |
| 49 | 134 |
| 50 | 86 |
| 51 | 95 |
| 52 | 64 |
| 53 | 60 |
| 54 | 305 |
| 55 | 118 |
| 56 | 95 |
| 57 | 918 |
| 58 | 216 |
| 59 | 76 |

### Example 5

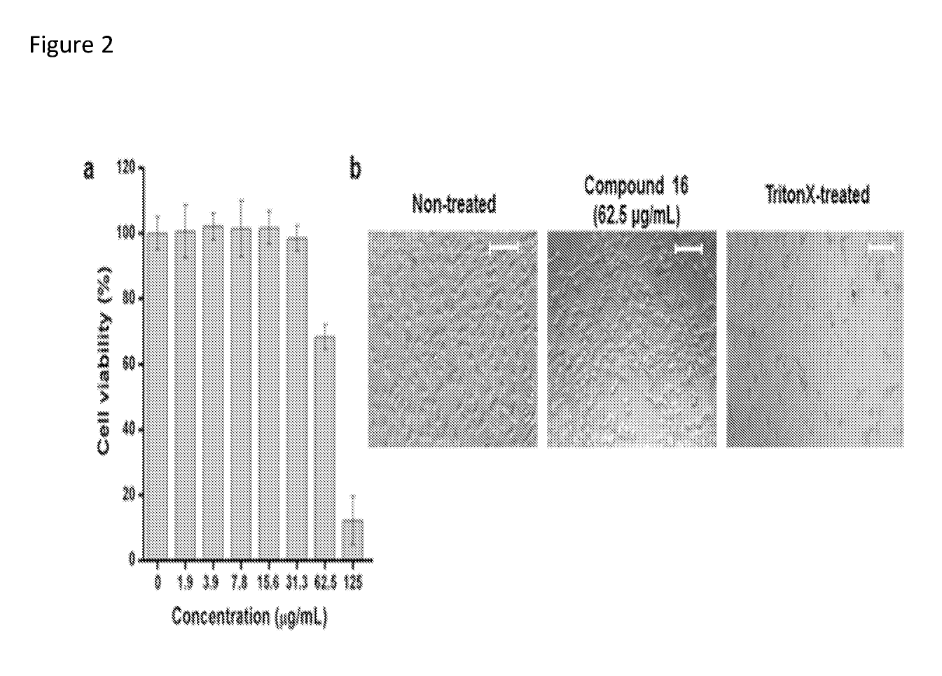

Cytotoxicity: Toxicity of the compounds against a mammalian cell line (Hela Cells) was determined using the MTT assay. Cytotoxicity of the compound 8a was assessed against HeLa and RAW (macrophage) cell lines. Briefly, the cells were grown in a 96-well plate in DMEM media (supplemented with 10% fetal bovine serum and 5% penicillin-streptomycin) until they reached around 70-80% confluency. The cells were then treated with 50 μL of serially diluted compound 16. Two controls were made; one containing no compound (non-treated cells) and the other one was treated with 10 vol % Triton-X 100 solution. The plate was incubated for 1 h at 37° C. under 5% CO₂ atmosphere. After 24 h, the medium was carefully removed and 100 μL of MTT solution (5 mg/mL concentration) was then added to each well. The plate was incubated for 4 h at 37° C. under 5% CO₂ atmosphere. Then it was centrifuged at 1100 rpm for 5 min and the supernatant was removed. After that 100 μL of DMSO was added to solubilize formazan crystals. The O. D. of the plate was then recorded at 570 nm. Percentage of cell survival was calculated using the following equation: Cell viability (%)=(A_{treated}-A_{tritonX-treated})/(A_{non-treated}-A_{tritonX-treated})×100. Each concentration had triplicate values and the average of triplicate O. D. values were plotted against concentration followed by fitted with sigmoidal plot. From the curve the values were determined corresponding to 50% cell viability. For bright-field microscopic images, a 40× objective was used and images were captured using a Leica DM2500 microscope. Compound 16 did not show any toxicity at its MIC and showed EC₅₀ values (50% cells viability)>70 μg/mL against these cell lines (FIG. 2). The results suggest that compound 16 holds enormous potential to be developed as a selective antibacterial agent.

### Example 6

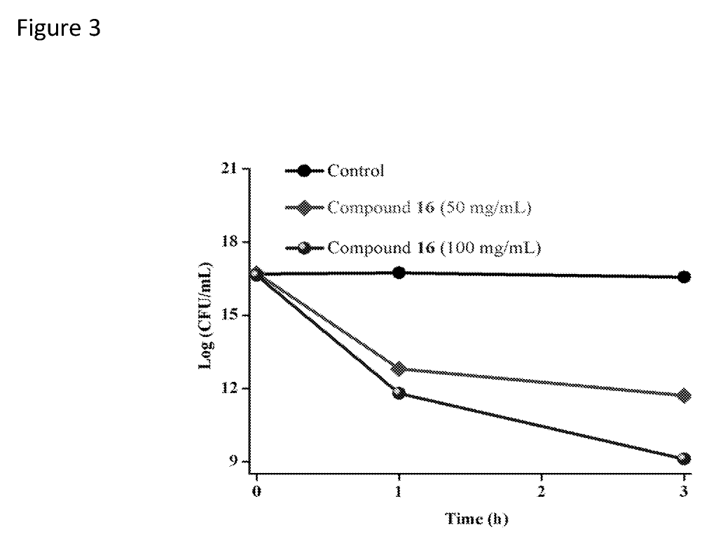

Antibacterial activity against stationary phase bacteria: As conventional antibiotics primarily target the cellular processes in bacteria, therefore most known antibiotics remain ineffective at the stationary-phase of bacteria. Time-kill kinetics against the stationary phase S. aureus bacteria were also investigated for compound 16. Briefly, S. aureus was grown in yeast-dextrose broth at 37° C. for 18 h to achieve them in stationary-phase. The test compound 16 was then added to the stationary-phase bacteria with the working concentrations of 50 and 100 μg/mL. It was then incubated at 37° C. with shaking at 150 rpm. At different time intervals (0, 1 and 3 h) 20 μL of aliquots from that solution were serially diluted 10-fold in 0.9% saline. Then from the dilutions, 20 μL was plated on yeast-dextrose agar plates and incubated at 37° C. After 24 h the bacterial colonies were counted and results represented in logarithmic scale, i.e. Log (CFU/mL). As shown in FIG. 3, compound 16 displayed more than 3 Log CFU/mL reductions in cell viability within 1 h of exposure at concentration even as low as 50 μg/mL (approximately 8×MIC of compound concentration). These results suggest that compound 16 retain high potency even at non-dividing stationary phase of bacteria where most of antibiotics remain ineffective at this phase of bacteria.

### Example 7

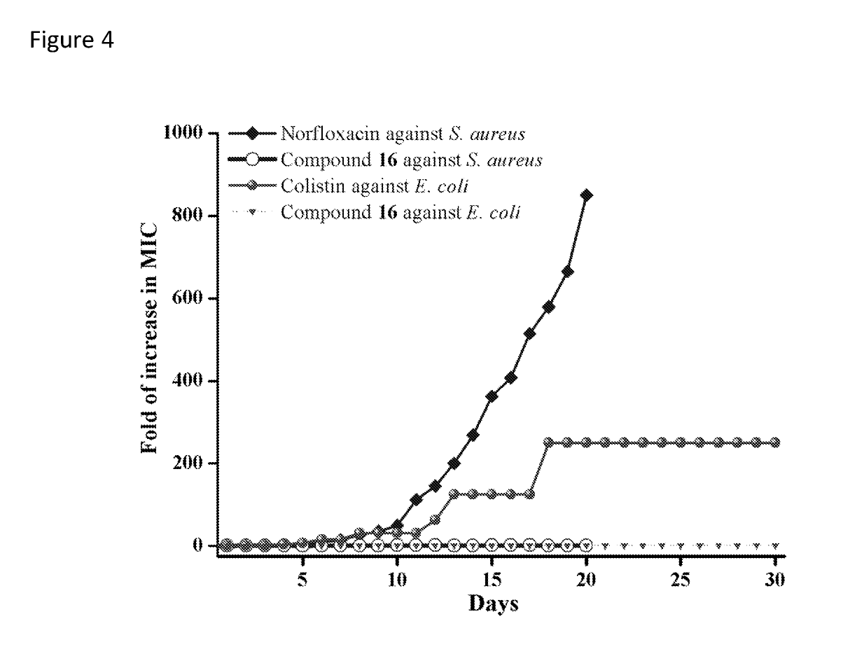

Resistance studies: The emergence of antibiotic-resistant bacteria is a major problem to global health. Hence, to investigate the potential of these compounds as an antibacterial agent with sufficient longevity, the ability of S. aureus (Gram-positive representative) and E. coli (Gram-negative representative) bacteria to develop resistance against these compounds was investigated. For this study, compound 16 was chosen as the model compound. Norfloxacin was used as a positive control for S. aureus, whereas colistin was used in the case of E. coli. At first, the MIC value of compound 16 was determined against both the bacteria. In the cases of norfloxacin and colistin the initial MIC values were also determined against respective bacteria. For the next day MIC experiment, the bacterial dilution was made by using the bacteria from sub-MIC concentration of the compounds (at MIC/2). After a 24 h incubation period, again bacterial dilution was prepared by using the bacterial suspension from sub-MIC concentration of the compound (at MIC/2) and assayed for the next MIC experiment. The process was repeated for 20 and 30 passages in the cases of S. aureus and E. coli, respectively. The fold of MIC increased for test compound, and control antibiotics were plotted against the number of days and it showed no change in the MIC for test compounds against S. aureus and E. coli even after 20 and 30 passages, respectively (FIG. 4), whereas around 800-fold increase in the MIC was observed in the case of norfloxacin and 250-fold in the case of colistin. This study suggested that bacteria find it difficult to develop resistance against this type of compound.

### Example 8

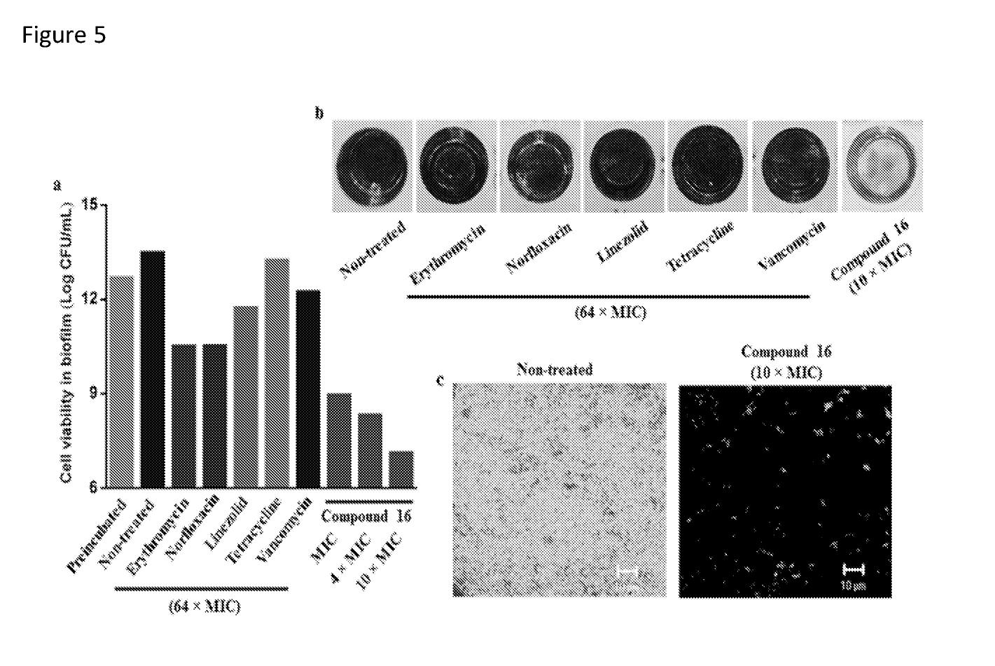

Bacterial biofilm disruption: Biofilms are adherent communities of bacteria where they are embedded within self-produced extracellular matrix consisting of exopolysaccharides, proteins and sometimes extracellular DNA. Bacteria behave as multicellular organisms inside the biofilm and develop strategies that prevent the entrance of antibiotics. As a result, antibiotics that are able to kill planktonic bacteria, are often ineffective to treat biofilm associated infections such as lung infections of cystic fibrosis (CF) patients, burn wound infections, catheter infections, bacterial endocarditis, chronic wound infections, and so on. The efficacy of this class of compounds as bacterial biofilm disrupting agent was established by comparing the extent of pre-formed biofilm eradication of compound 16 and antibiotics such as erythromycin, norfloxacin, linezolid, tetracycline and vancomycin. Briefly, stock solution was prepared with midlog phase culture of S. aureus (diluted to approximately 10⁵ CFU/mL) in nutrient broth supplemented with 1% glucose and 1% NaCl. Biofilms were then grown into a 96-well plate by adding 100 μL of this stock solution. Then, 100 μL of the test compound 16 and control antibiotics such as erythromycin, norfloxacin, linezolid, tetracycline and vancomycin (64×MIC) were added to the biofilm and allowed to incubate for 24 h. One control was made where 100 μL of complete medium was added instead of compound or antibiotics. At the end of 24 h disrupted biofilms were quantified by plating serial 10-fold dilutions of biofilm suspension. Cell viability in biofilms was expressed as Log (CFU/mL) and compared with the non-treated control. For visualizing, disrupted biofilms were stained with 0.1% of crystal violet (CV) and images were captured using normal digital camera. For confocal laser-scanning microscope, biofilm were stained with SYTO-9 (3 μM). Results suggested that compared to the non-treated control, compound 16 displayed significant reductions (more than 4 Log CFU/mL) of cell viability in the pre-existing biofilms of pathogenic S. aureus bacteria at its MIC concentration, whereas none of the antibiotics showed significant reduction even at very high concentrations (64×MIC) as shown in FIG. 5a. Furthermore, 5 and 6 Log (CFU/mL) reduction in cell viability was observed at concentrations of 4×MIC and 10×MIC, respectively. Crystal violet staining and confocal imaging, allowed us to visualize the extent of biofilm disruption. Crystal violet staining clearly illustrated that compound 16 (at a concentration of 10×MIC) displayed an enormous reduction in pre-formed biofilm mass compared to the non-treated control and the antibiotics as shown in FIG. 5b. At the same concentration, confocal microscopy also revealed significant biofilm eradication as shown in FIG. 5c.

### Example 9

### In-Vitro Antiviral Activity

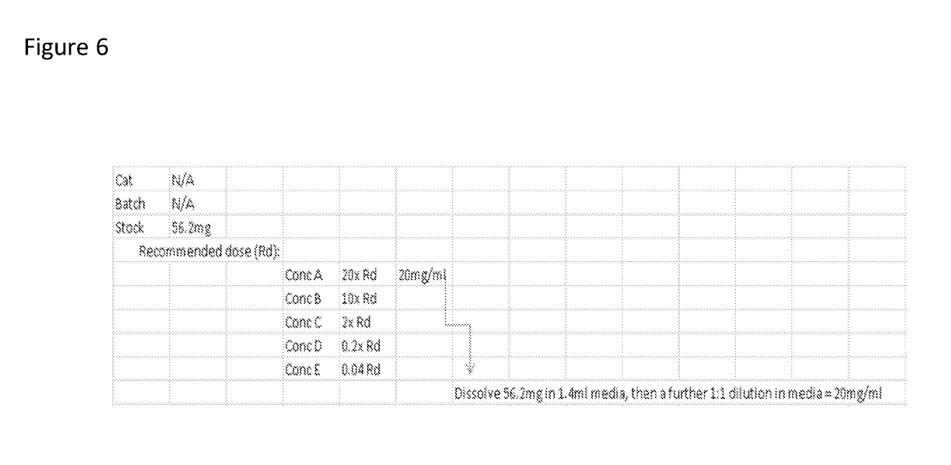

MRC-5 and VeroE6 cells were sourced from the European Collection of Cell Cultures having been seeded into 96-well plates. Compound 16 was selected as a model for in vitro antiviral studies. A range of concentrations for were made at two levels either side of the recommended concentration suggested for in vitro work (1 mg/ml). Concentrations were made at double the final dilution to take into account an equal volume of virus suspension to be added (FIG. 6).

Within a Containment Level 4 laboratory, media was removed from the inner wells of the 96-well plates. Due to edge-effects, the outer wells were left with media added. Compounds were added at 5 replicates per dilution. Ebola virus suspension (strain ME718) was added at a concentration of approximately 500 TCID50/well to triplicate wells per compound dilution, with the remaining two wells having media alone added.

Based on results from growth of Ebola virus in MRC-5 and VeroE6 cells, the supernatants from MRC-5 and VeroE6 cells were harvested on days 3 and 6 post-infection, respectively. Cells were microscopically assessed for assessment of cell health. 140 μl of supernatant was added to 560 μl AVL buffer for RNA extraction and PCR. Ct values from the PCR assay were used to give a consistent reading of the amount of Ebola virus RNA levels in the samples. Formaldehyde solution was added to all wells to fix attached cells for subsequent staining with crystal violet to assess cell attachment.

To confirm these results, a second in vitro study was conducted in MRC-5 cells. The Ebola virus infection was at a higher inoculum (5000 TCID50 per well) and supernatants harvested after 2 days into AVL buffer. Additionally, the surfaces of the wells were treated with RLT buffer and samples collected for the assessment of viral RNA levels within attached cells. Additionally, concentrations of compounds used were lower.

The Ct values from the Ebola virus PCR assays from the first in vitro testing are shown in Tables 7 and 8 from the MRC-5 and VeroE6 studies, respectively. The PCR assay showed undetectable Ebola virus specific signals at high concentrations and then reductions in viral genome levels at lower concentrations.

TABLE 7
Ct values from MRC-5 cell supernatant
3 days post-Ebola virus infection.

| | | | | | | Drug - no drug |
|---|---|---|---|---|---|---|
| Compound 16 | 1 | 2 | 3 | Ave | Std Dev | Ct change |
| 10x | UD | UD | UD | — | — | — |
| 5x | UD | UD | UD | — | — | — |
| 1x | UD | UD | UD | — | — | — |
| 0.1x | 34.79 | 33.84 | 34.37 | 34.3 | 0.48 | 7.02 |
| 0.02x | 32.60 | 32.79 | 33.47 | 32.9 | 0.46 | 5.64 |
| 0 | 26.17 | 26.64 | 27.71 | 26.8 | 0.79 | |

| | | |
|---|---|---|
| Cell plate controls | 27.3 | 1.88 |
| no drug | | |

“UD” means undetermined.

TABLE 8
Ct values from VeroE6 cell supernatant
6 days post-Ebola virus infection.

| | | | | | | Drug - no drug |
|---|---|---|---|---|---|---|
| Compound 16 | 1 | 2 | 3 | Ave | Std Dev | Ct change |
| 10x | UD | Not | UD | — | — | — |
| | | done | | | | |
| 5x | UD | UD | UD | — | — | — |
| 1x | UD | UD | UD | — | — | — |
| 0.1x | 34.00 | UD | 33.30 | 33.65 | 0.50 | 3.76 |
| 0.02x | 33.87 | 32.88 | 36.70 | 35.29 | 2.00 | 5.40 |
| 0 | 28.22 | 35.03 | 27.96 | 28.09 | 0.18 | |

| | | |
|---|---|---|
| Cell plate controls | 29.89 | 3.43 |
| no drug | | |

“UD” means undetermined.

To confirm the antiviral effects of model compound 16, the in vitro screening was repeated in the MRC-5 cells infected with a higher dose of Ebola virus and left for 2 days. Ct values from the supernatant and attached cells are shown in tables 9 and 10, respectively. The Ct values are generally lower than the previous study, most likely as a result of the higher viral inoculum used. Results were consistent with the previous study, showing that and compound 16 reduced the amount of viral genome present.

TABLE 9
Ct values from MRC-5 cell supernatants
2 days post-Ebola virus infection.

| | | | | | | Drug-no drug |
|---|---|---|---|---|---|---|
| Compound 16 | 1 | 2 | 3 | Ave | StdDev | Ct change |
| 60 ug/ml | 26.99 | 26.76 | 27.13 | 26.97 | 0.19 | 1.49 |
| 20 ug/ml | 26.30 | 26.31 | 26.49 | 26.37 | 0.11 | 0.90 |
| 6.67 ug/ml | 26.53 | 26.04 | 26.63 | 26.40 | 0.32 | 0.93 |
| 2.22 ug/ml | 25.96 | 27.13 | 26.42 | 26.51 | 0.59 | 1.04 |
| 0.74 ug/ml | 26.03 | 24.81 | 25.73 | 25.53 | 0.64 | 0.06 |
| 0 | 25.41 | 25.55 | 25.44 | 25.47 | 0.08 | 0.00 |

TABLE 10
Ct values from MRC-5 cells 2 days post-Ebola virus infection.

| | | | | | | Drug-no drug |
|---|---|---|---|---|---|---|
| Compound 16 | 1 | 2 | 3 | Ave | StdDev | Ct change |
| 60 ug/ml | 24.42 | 24.33 | 24.32 | 24.36 | 0.05 | 6.77 |
| 20 ug/ml | 21.29 | 20.72 | 23.10 | 21.71 | 1.24 | 4.13 |
| 6.67 ug/ml | 19.95 | 20.87 | 20.22 | 20.35 | 0.47 | 2.77 |
| 2.22 ug/ml | 17.82 | 20.49 | 19.45 | 19.26 | 1.35 | 1.68 |
| 0.74 ug/ml | 16.70 | 18.76 | 16.93 | 17.47 | 1.13 | −0.12 |
| 0 | 17.59 | 18.04 | 17.10 | 17.58 | 0.47 | 0.00 |

A further in vitro study was performed in which model compound 16 was compared with 19 other compounds, which were identified according to their Technology Readiness Score, their ability to make a difference to the current Ebolavirus epidemic, and their likely efficacy against Ebola virus. The effects of these 20 compounds (including compound 16) on viral amplification was assessed using MRC-5 and VeroE6 cells infected with Ebola virus. Of these 20 compounds, compound 16 provided one of the top two Ct values, in both cell types.

The above mentioned implementation examples as described on this subject matter and its equivalent thereof have many advantages, including those which are described.

The compounds disclosed in the present disclosure are highly stable in human plasma. The compounds of the present disclosure show high antibacterial activity against various pathogens including drug resistant bacteria. Toxicity studies showed that all compounds are selective towards bacteria over human RBCs. The compounds in the present disclosure also show high antiviral activity, even against highly pathogenic virus. Thus, the compounds in the present disclosure exhibit highly advantageous antimicrobial properties. In view of the advantageous antibacterial and antiviral results described above, the inventors believe that the compounds of the present disclosure may also be used against other types of microbe, particularly fungi and protozoa.

Although the subject matter has been described in considerable detail with reference to certain embodiments thereof, other embodiments are possible. As such, the spirit and scope of the invention should not be limited to the description of the embodiments contained herein.

1. A compound of formula I 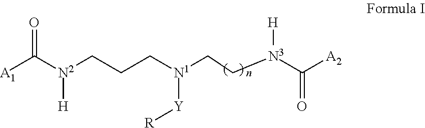
or its stereoisomers, pharmaceutically acceptable salts, polymorphs, solvates and hydrates thereof, wherein,
n is 1, 2, 3, 4, or 5; Y is selected from the group of-CH₂—, —CO—, —CONH—, —COO—;
A₁ and A₂ are independently selected from a sequence of one or up to 4 additional amino acids, wherein the amino acids are independently selected from L-configuration or D-configuration, wherein A₁ and A₂ are optionally substituted with one or more of R₂;
R is selected from C6-C28alkyl, C4-C28alkenyl, C4-C28alkyne, C6-C18aryl, C3-C28cycloalkyl, saturated or unsaturated 5 to 18 membered heterocyclyl, with 1, 2 or 3 hetero atoms selected from O, N and S, wherein R is optionally substituted with one or more of R₂;
R₂ is independently selected from the group consisting of hydrogen, halogen, CF₃, CN, straight or branched C1-C6alkyl, C3-C6cycloalkyl, C5-C6 aryl, aromatic 5 to 6 membered heterocyclyl, with 1, 2 or 3 hetero atoms selected from O, N and S;
and wherein the compound exhibits anti-microbial activity.

2. The compound as claimed in claim 1 or its stereoisomers, pharmaceutically acceptable salts, polymorphs, solvates and hydrates thereof wherein, Y is —CH2- or —CO—;
A₁ and A₂ are same or different, and independently selected from 2 amino acid residues, wherein the amino acids are independently selected from L-configuration or D-configuration; R is selected from C4-28 alkyl, C6-18 aryl.

3. The compound as claimed in claim 1 or its stereoisomers, pharmaceutically acceptable salts, polymorphs, solvates and hydrates thereof wherein,
A₁ and A₂ are same or different, and independently selected from 1, 2, 3, or 4 amino acid residues wherein the amino acid residues are independently selected from L-configuration or D-configuration and are positively charged.

4. The compound as claimed in claim 1 or its stereoisomers, pharmaceutically acceptable salts, polymorphs, solvates and hydrates thereof wherein,
Y is —CH₂—;
N¹ is positively charged.

5. A compound as claimed in claim 1 or its stereoisomers, pharmaceutically acceptable salts, polymorphs, solvates and hydrates thereof, which is selected from a group consisting of:

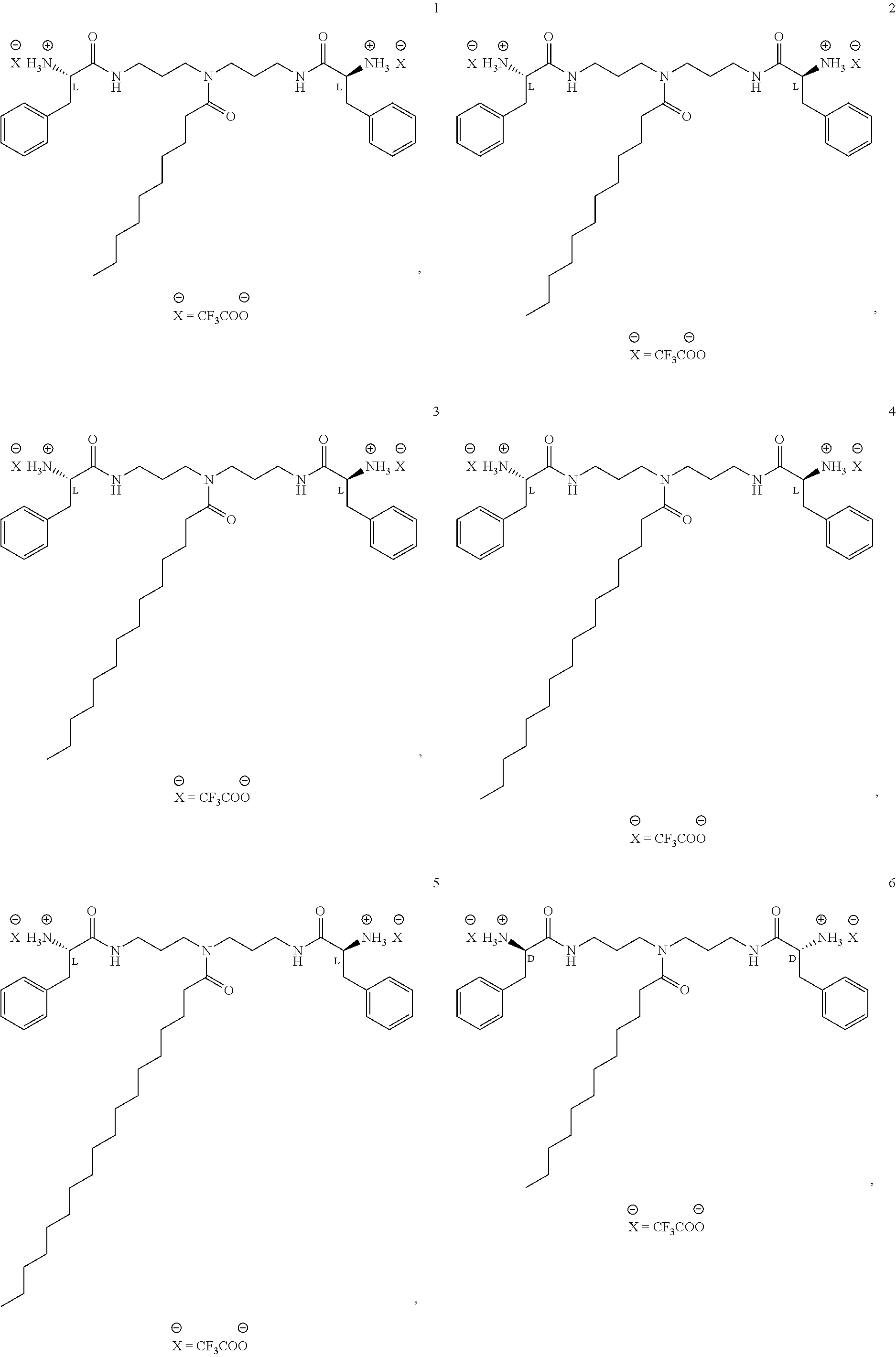

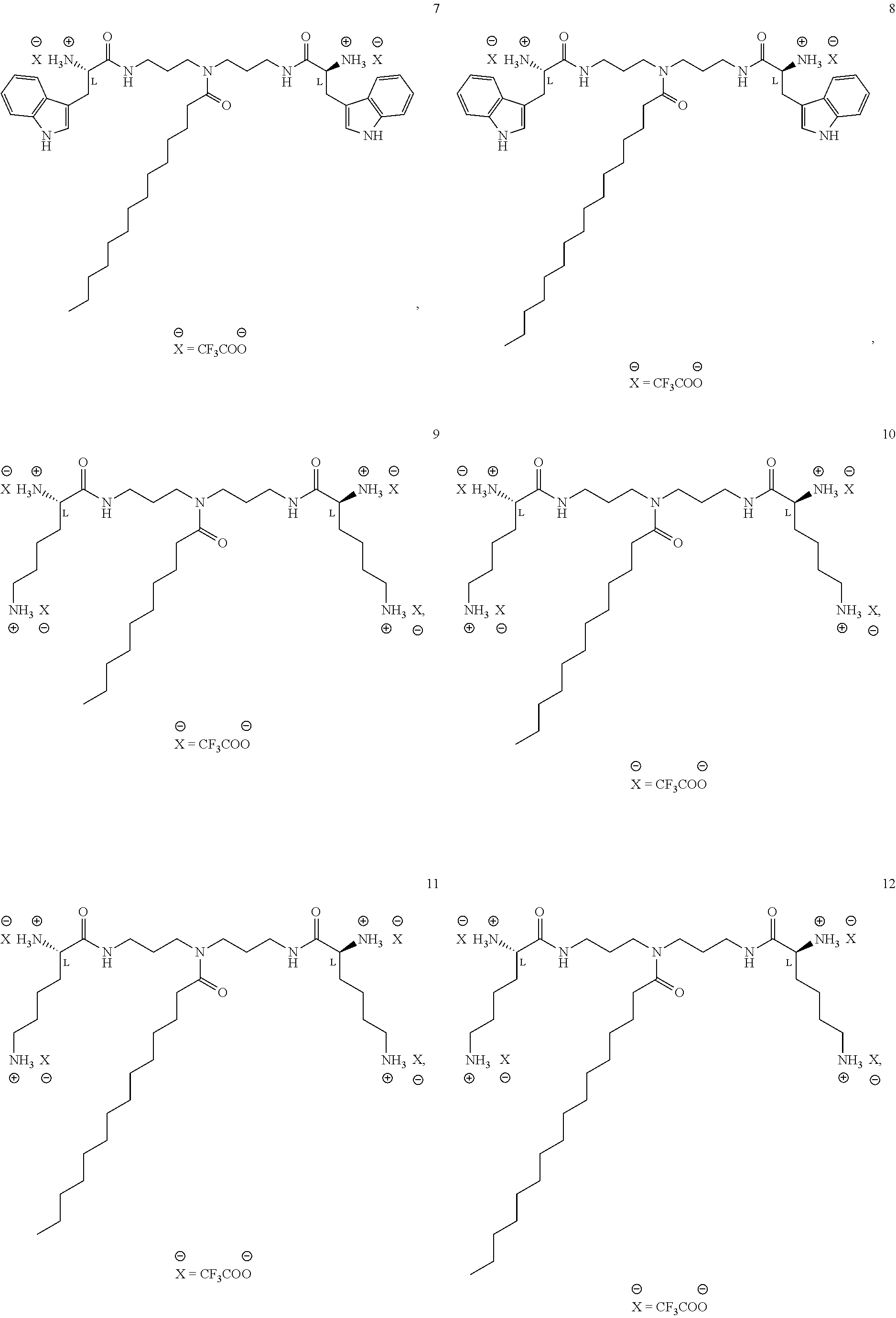

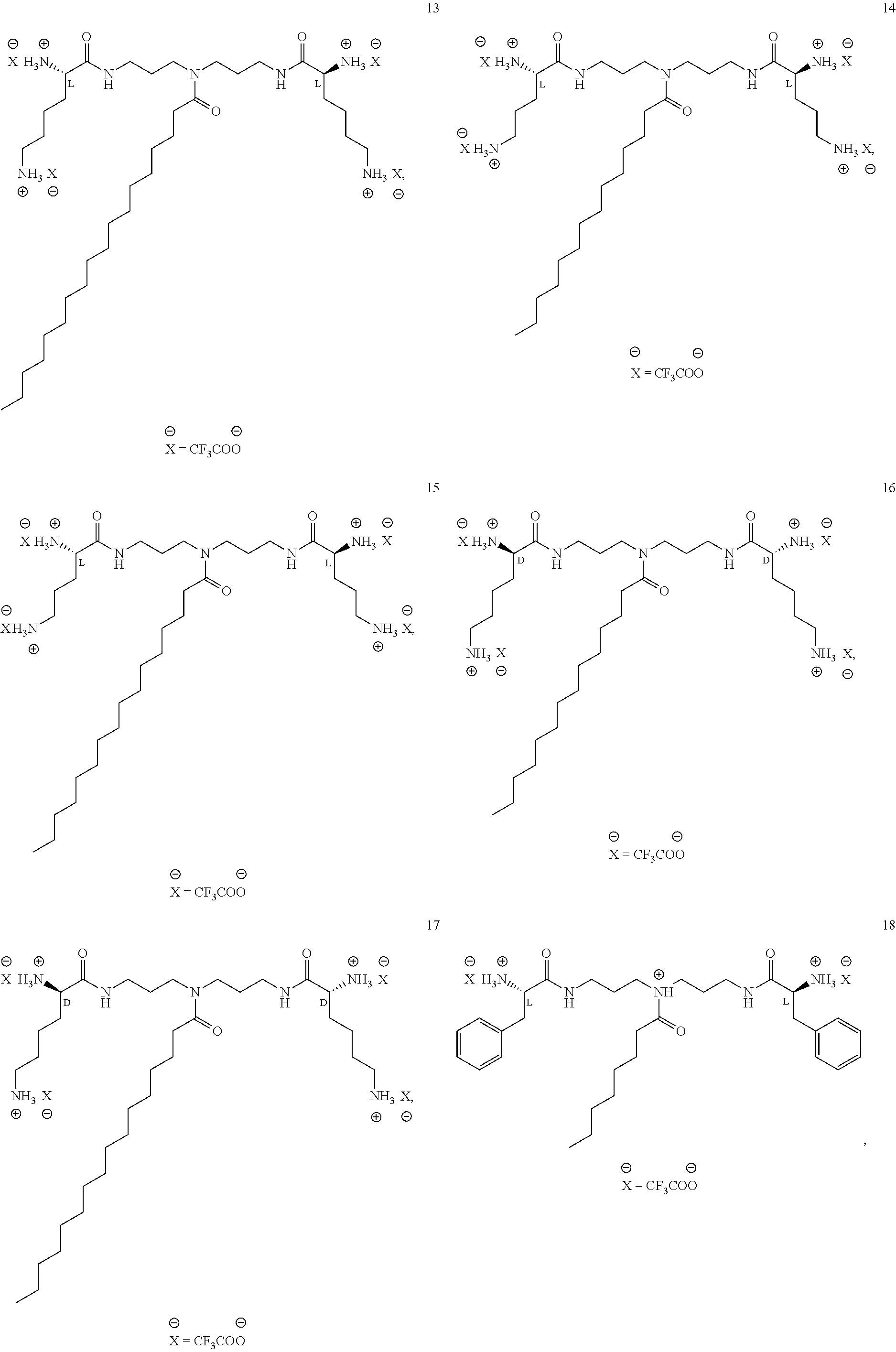

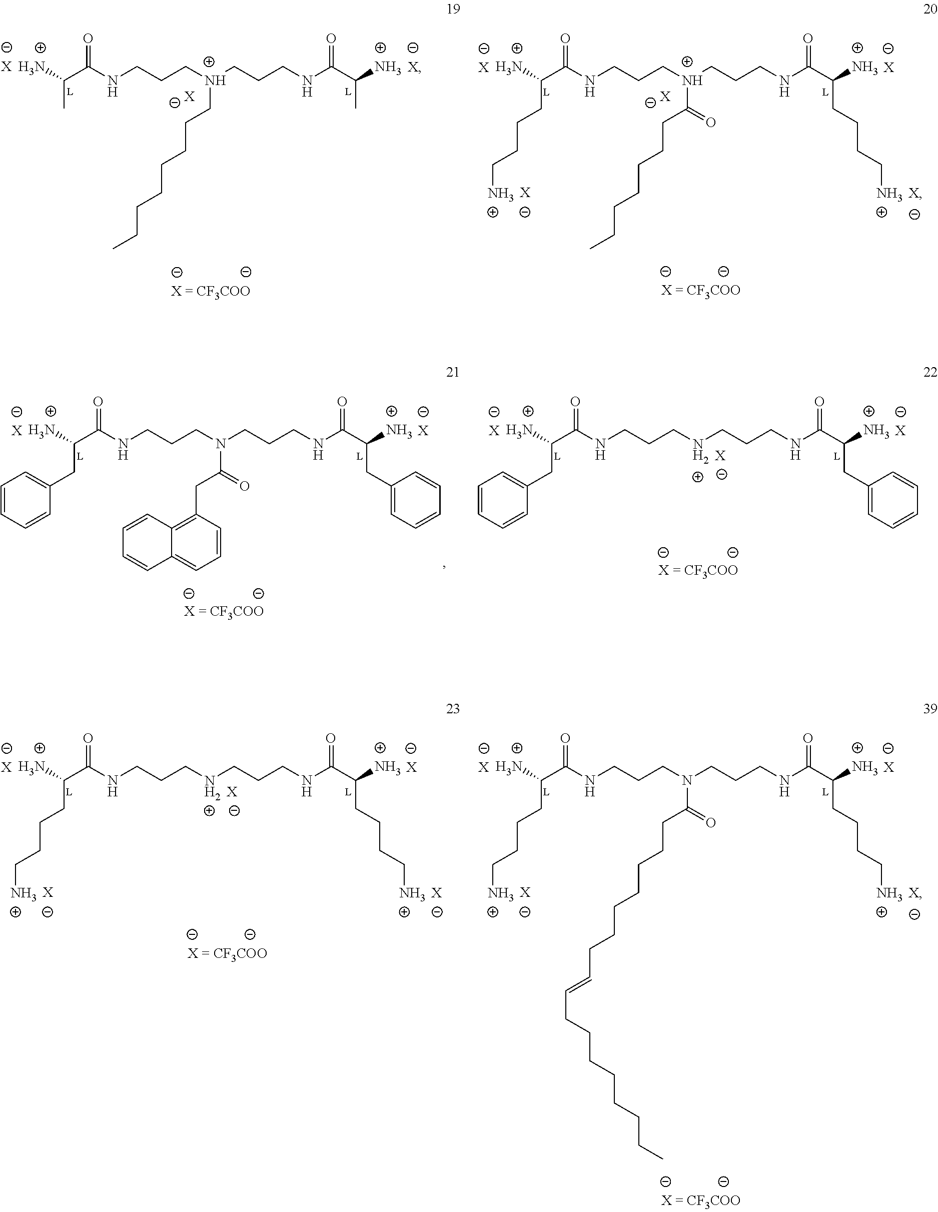

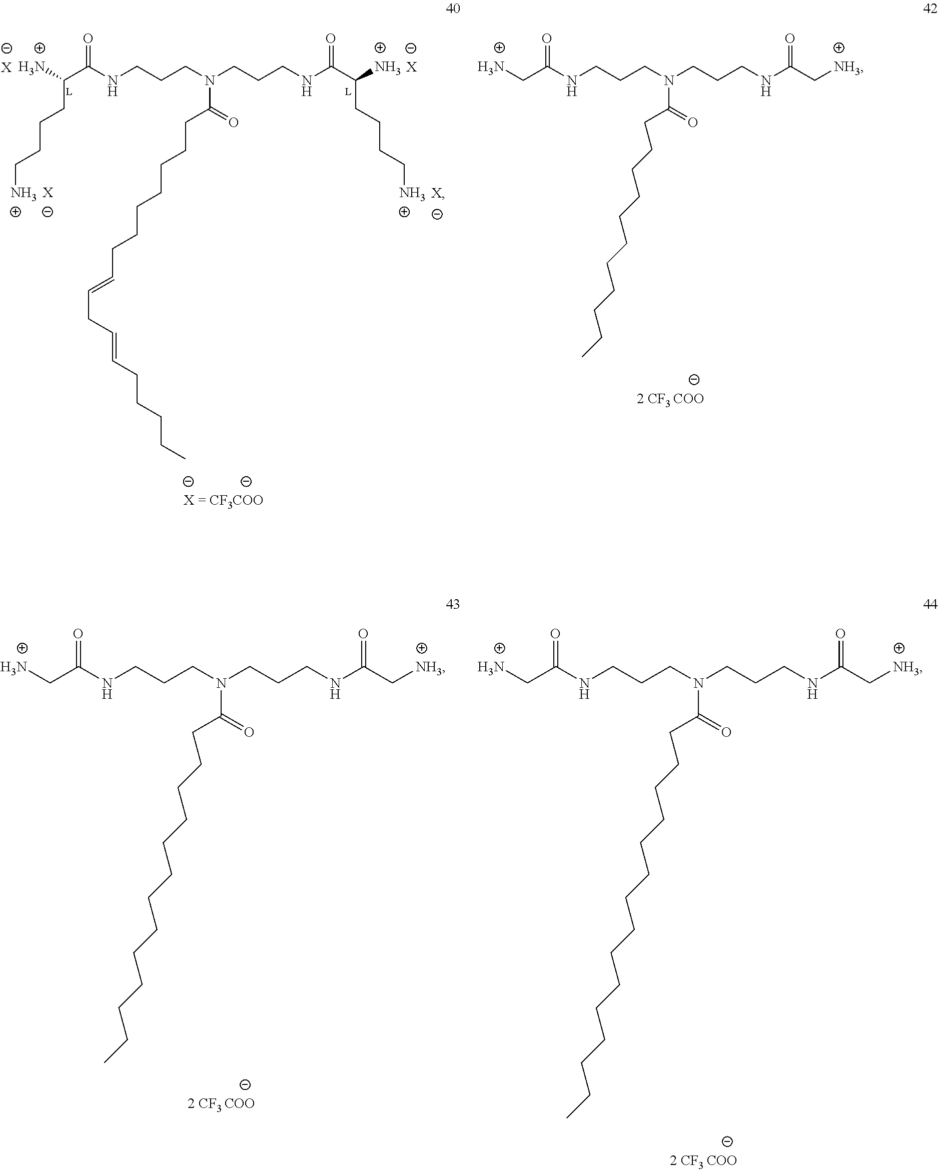

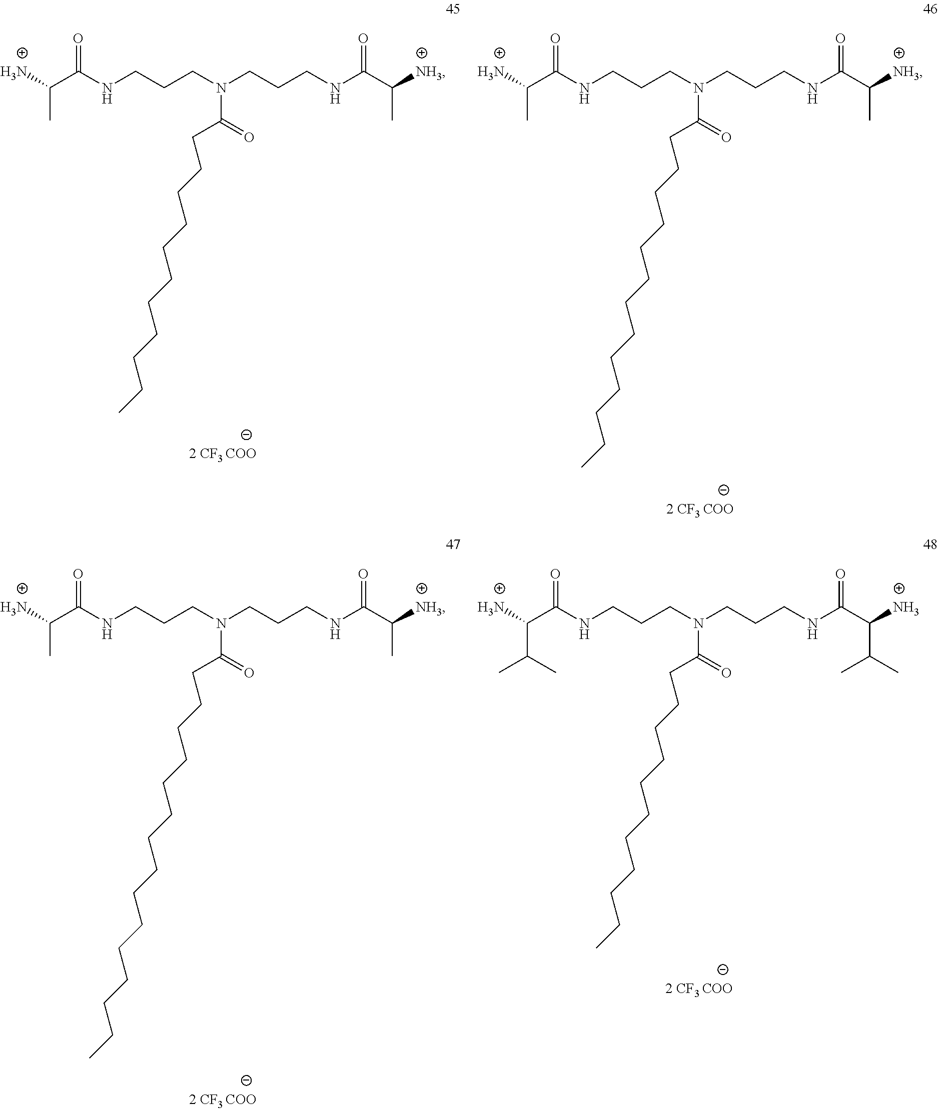

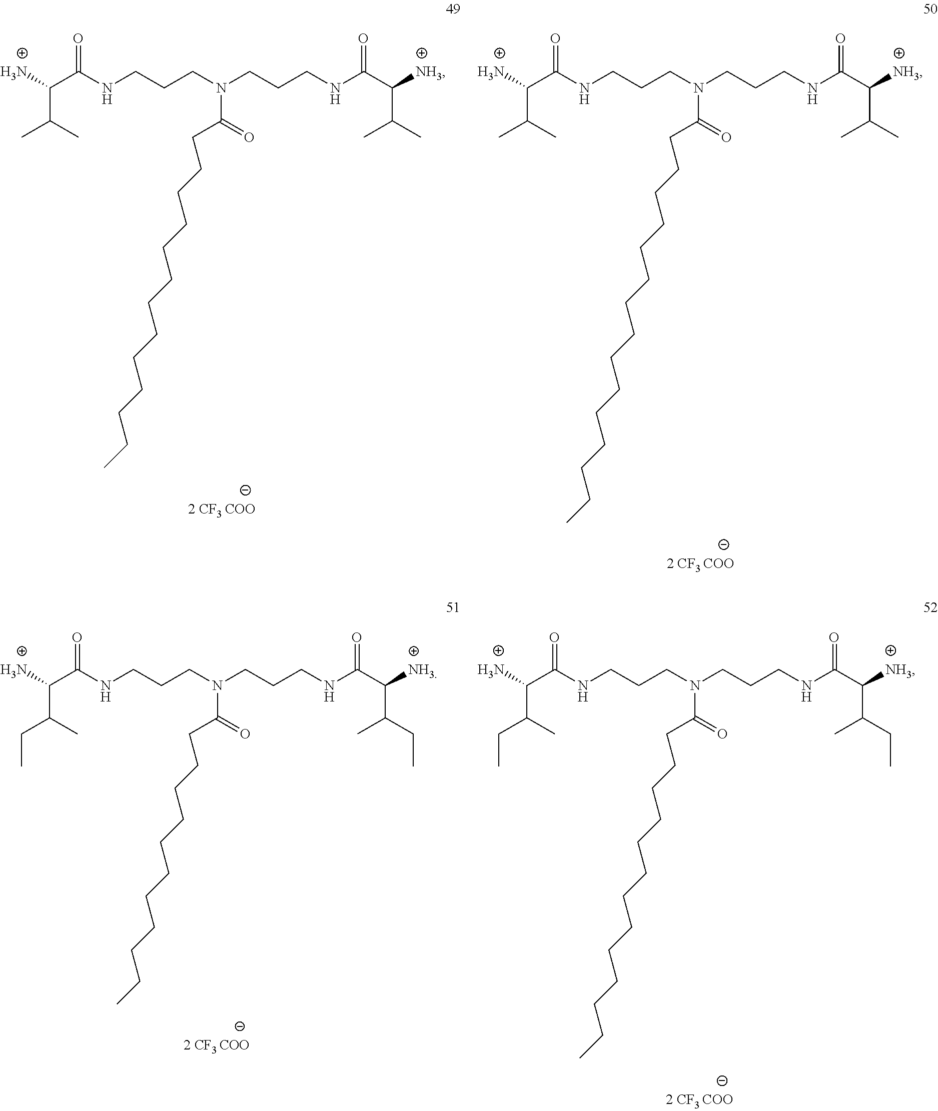

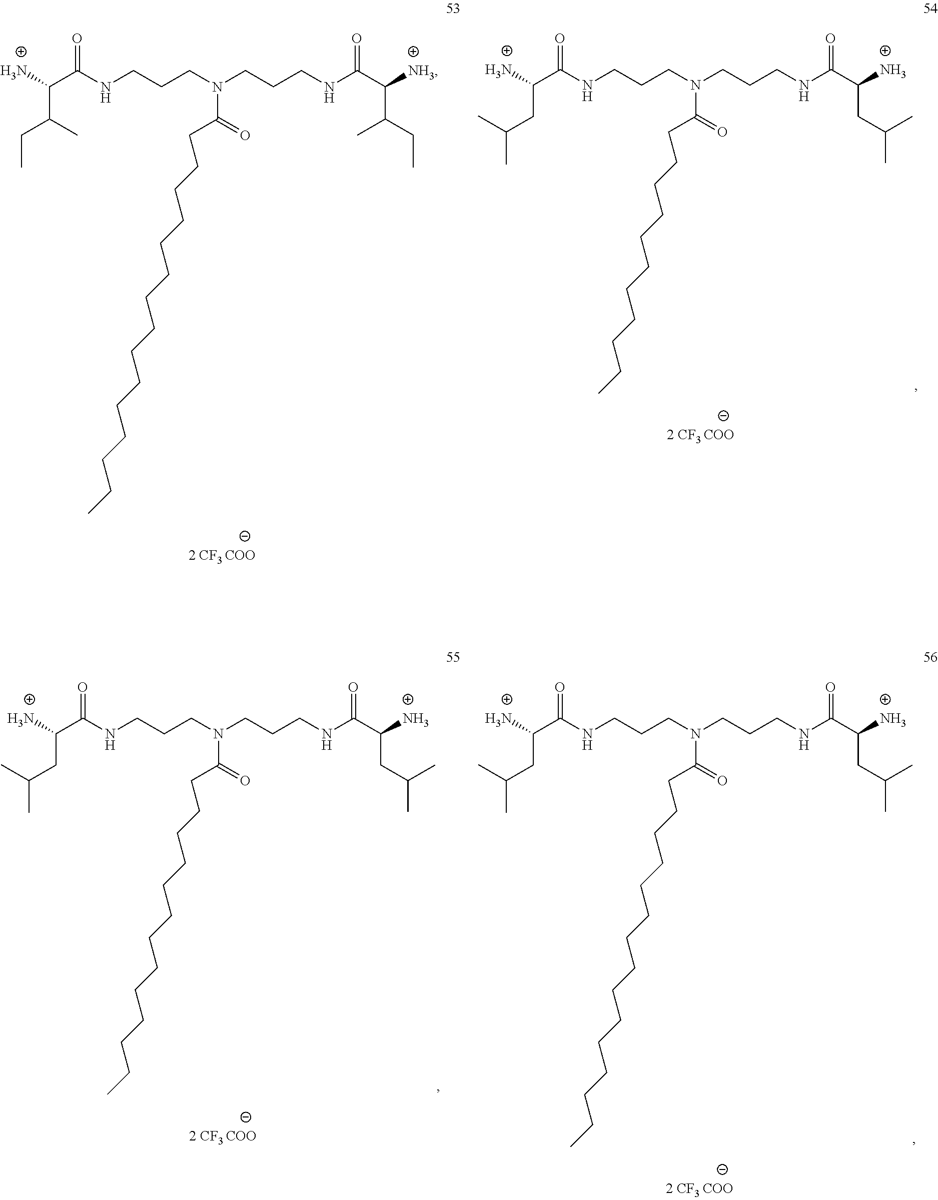

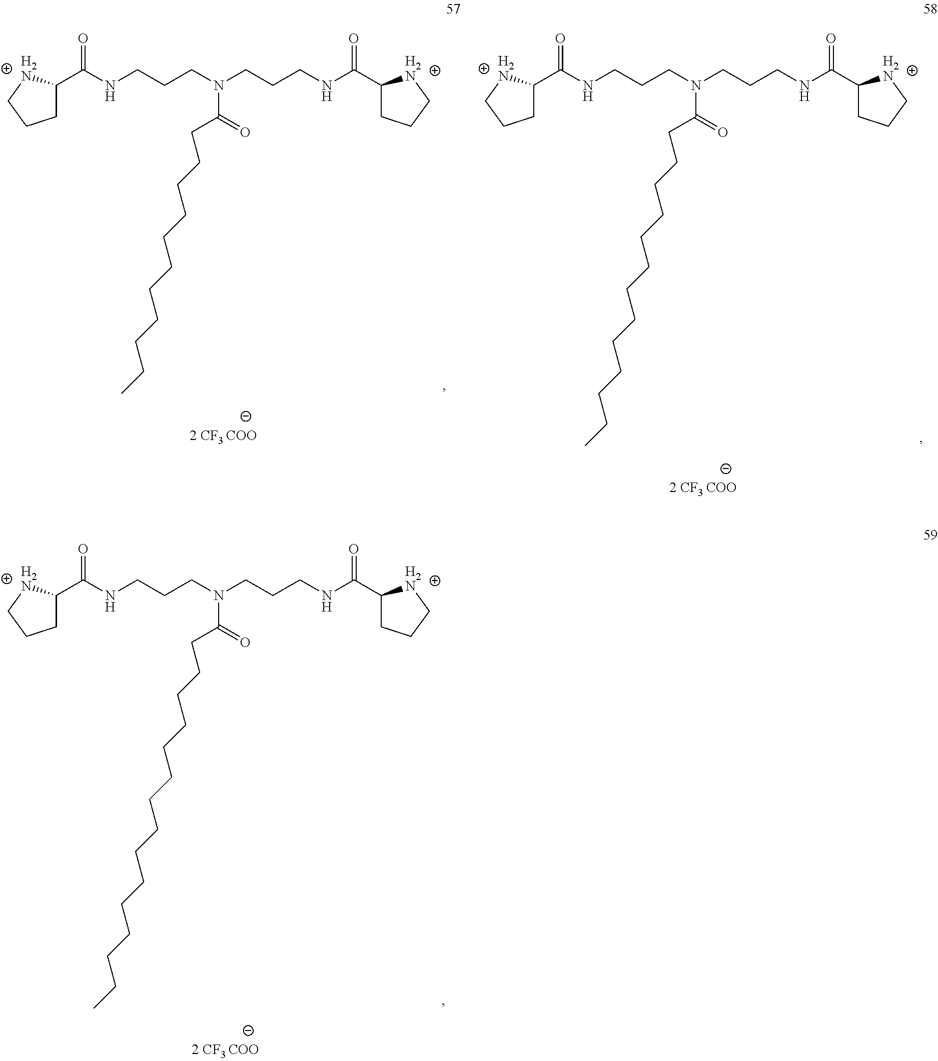

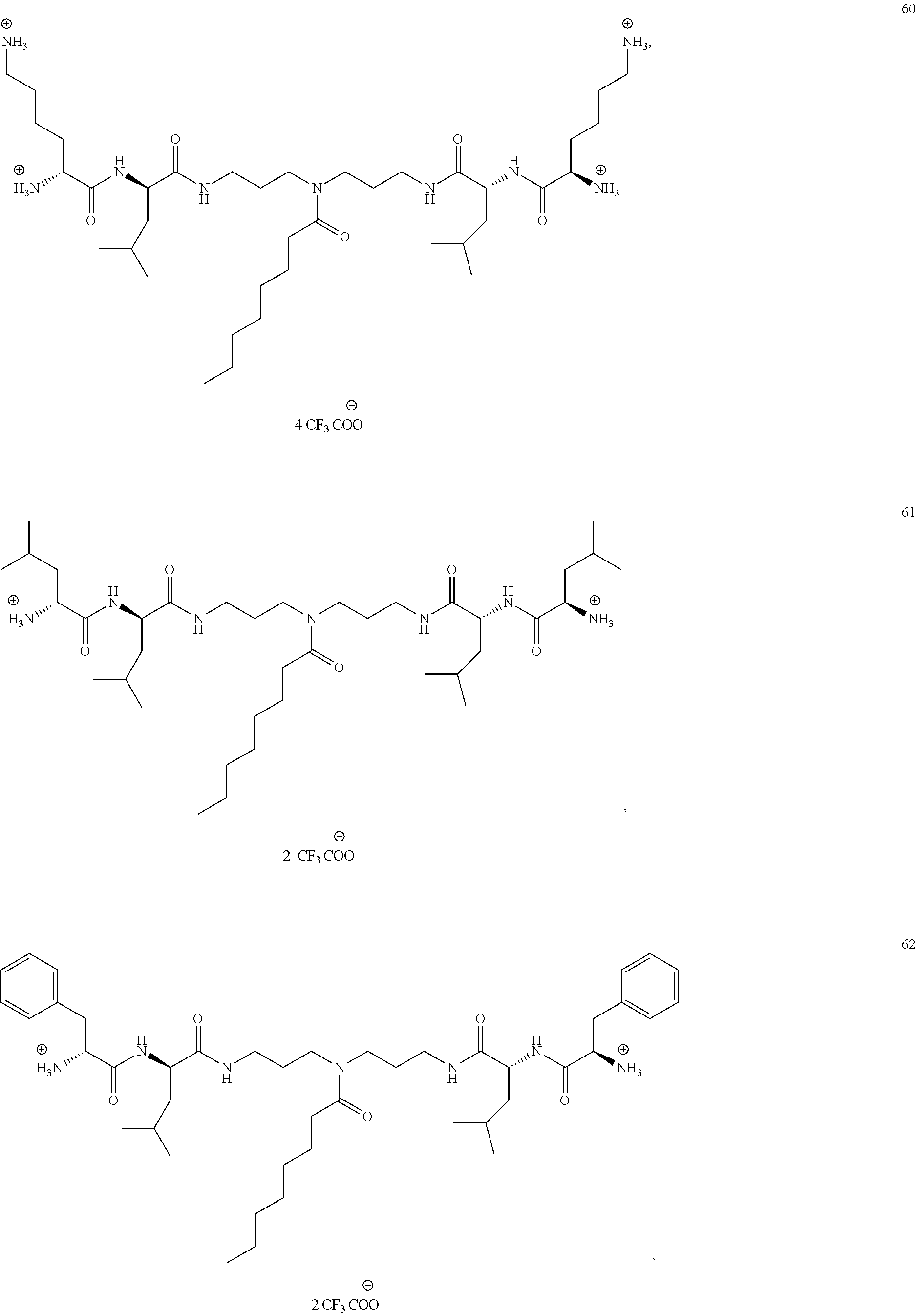

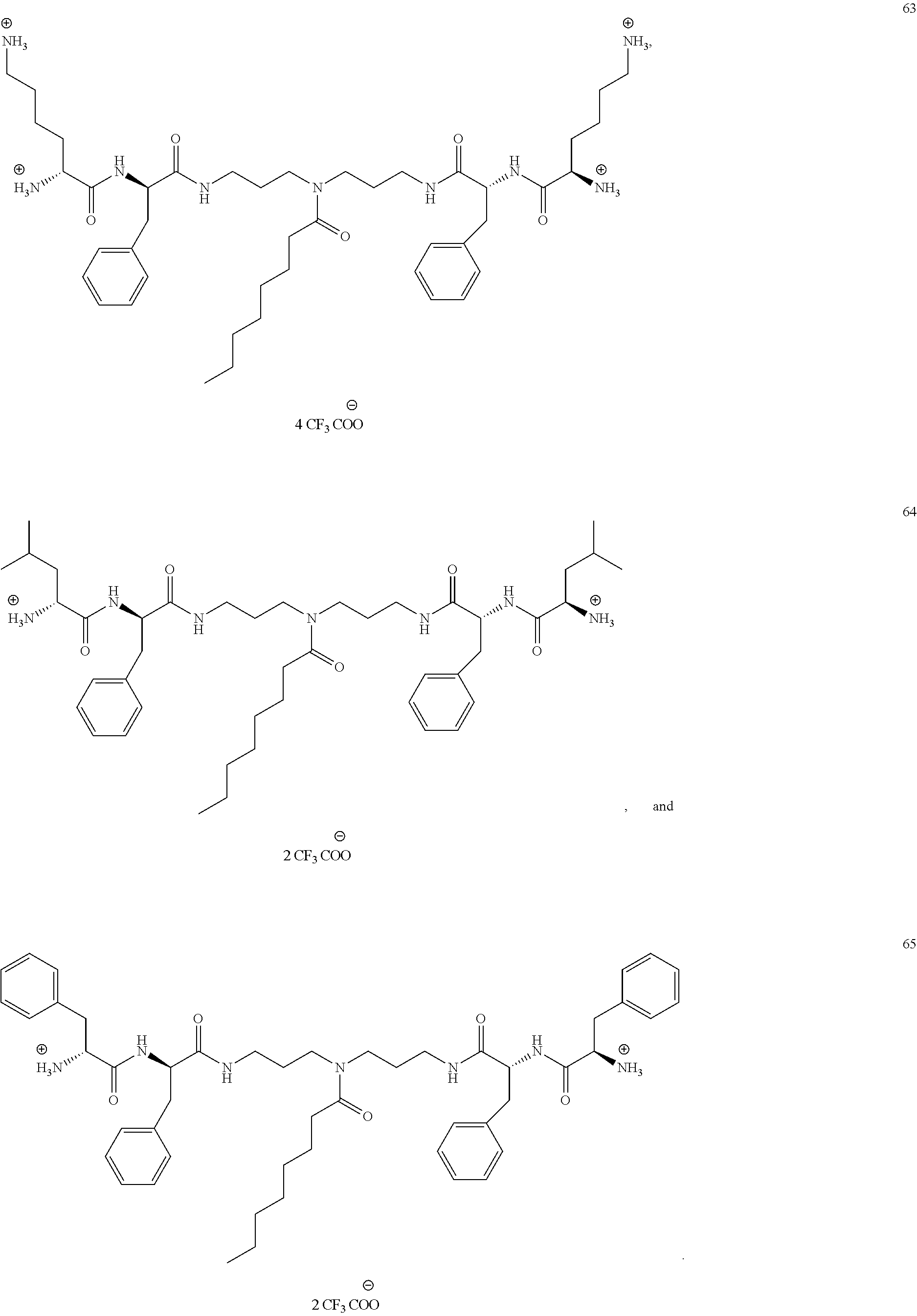

6. A compound as claimed in claim 1 or its stereoisomers, pharmaceutically acceptable salts, polymorphs, solvates and hydrates thereof, wherein said compound is:

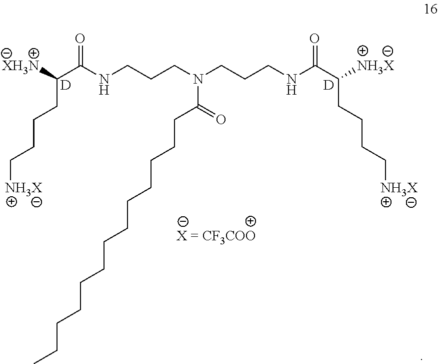

7-10. (canceled)

11. A method of treating a disease or condition in a patent, said method comprising administering to a patient a compound of formula (I), as claimed in claim 1, or its stereoisomers, pharmaceutically acceptable salts, polymorphs, solvates and hydrates thereof, wherein said disease or condition is caused by microorganism selected from the group consisting of bacteria, virus, fungi and protozoa

12. The method of claim 11, wherein the microorganism is selected from the group consisting of bacteria, virus and fungi.

13. The method of claim 12, wherein the microorganism is selected from the group consisting of bacteria and virus.

14. The method of claim 11, wherein the microorganism is virus.

15. The method of claim 14, wherein the virus is Ebola virus.

16. The method of claim 11, wherein the bacteria is a Gram-positive bacteria.

17. The method of claim 11, wherein the bacteria is a Gram-negative bacteria.

18. The method of claim 11, wherein the bacteria is a drug sensitive bacterium selected from a group consisting of S. aureus, E. faecium and E. coli or any combinations thereof.

19. The method of claim 11, wherein the bacteria is a drug sensitive bacterium selected from a group consisting of vancomycin-resistant E. faecium, methicillin-resistant S. aureus and β-lactam resistant K. pneumoniae, or any combination thereof.

20. An antimicrobial coating or surface comprising a compound of formula (I) as claimed in claim 1, or its stereoisomers, pharmaceutically acceptable salts, polymorphs, solvates, and hydrates thereof.

21. The surface as claimed in claim 20, wherein the surface comprises a material selected from the group consisting of metals, ceramics, glass, polymers, plastics, fibers and combinations thereof.

22. A composition comprising a compound of formula (I) or a salt thereof of as claimed in claim 1, and a carrier.

23. A pharmaceutical composition comprising a compound of formula (I) or a pharmaceutically acceptable salt thereof of as claimed in claim 1 together with a pharmaceutically acceptable carrier, optionally in combination with one or more other pharmaceutical compositions.

24. (canceled)
